(19) United States
(12) Patent Application Publication
(10) Pub. No.: US 2022/0339285 A1
(43) Pub. Date: Oct. 27, 2022

(54) DISSACHARIDE FORMULATIONS FOR CONTROLLED DRUG RELEASE

(71) Applicant: TECHNICAL UNIVERSITY OF DENMARK, KGS. Lyngby (DK)

(72) Inventors: Thomas Lars ANDRESEN, Vanlose (DK); Jonas ROSAGER HENRIKSEN, Allerod (DK); Anders Elias HANSEN, Limhamn (SE); Carl Fredrik MELANDER, Bunkeflostrand (SE); Elizabeth SERRANO CHAVEZ, Kobenhavns (DK); Linda Maria BRUUN, Kobenhavns (DK)

(21) Appl. No.: 17/618,743

(22) PCT Filed: Jun. 12, 2020
(86) PCT No.: PCT/EP2020/066403

§ 371 Date: Dec. 13, 2021

(30) Foreign Application Priority Data
Jun. 12, 2019 (SE) 1950699-7

(51) Int. Cl. A61K 47/26; A61K 9/08; A61K 47/44; A61K 47/14; A61K 47/34; A61K 47/10; A61K 31/4745; A61K 31/4709; A61K 31/136; A61K 31/69; A61K 31/4545; A61K 31/704; A61K 31/7048; A61K 31/497; A61K 31/519; A61K 31/506; A61K 31/4375; A61K 9/06; A61K 49/04; A61K 51/12

(52) U.S. Cl. CPC A61K 47/26; A61K 9/08; A61K 47/44; A61K 47/14; A61K 47/34; A61K 47/10; A61K 31/4745; A61K 31/4709; A61K 31/136; A61K 31/69; A61K 31/4545; A61K 31/704; A61K 31/7048; A61K 31/497; A61K 31/519; A61K 31/506; A61K 31/4375; A61K 9/06; A61K 49/0438; A61K 51/1213

(57) ABSTRACT

The present invention relates to a composition comprising non-water soluble dissacharides and oil, a solvent and at least one pharmaceutical ingredient, wherein the composition contains at least two compounds selected from saccharides and lipid oils such as lactose octabenzoate Methyl hepta-O-isobutyryl-α,β-lactoside, α,β-Lactose octa para-iodobenzoate, 3-iodobenzyl hepta-O-isobutyryl-α,β-lactoside, lactose octapropionate, lactose octaisobutyrate, sucrose octabenzoate, glycerol trihexanoate, Glycerol trioctanoate, Glycerol tridecanoate, Lipiodol, ethyl myristate, ethyl palmitate, ethyl oleoate and wherein the composition is a liquid before administration into the human or animal body and increases in viscosity by more than 2,000 centipoise (cP) after administration.

### FIELD OF THE INVENTION

The present invention provides controlled release of therapeutics from disacharide formulations for treatment of disease.

### TECHNICAL BACKGROUND

Biomaterials for use as drug delivery systems have found wide interest for treatment of multiple diseases and conditions in humans and animals, such as pain, inflammation, infection, tissue regeneration and repair, allergy, and cancer. The present invention provides injectable liquid compositions that gels or solidifies after administration to human or animal body after which it provides a system for controlled drug release.

Other patents and articles have described the use of biomaterials for controlled release of drugs for various applications. EP1212092 and U.S. Pat. No. 6,413,536 describe formulations for drug delivery based on a hydrophobic gel matrix consisting of organic solvent, a saccharide ester based on sucrose derivatives such as SAIB or other poly-ols and one or several drugs. The injectable formulations are based on derivatized carbohydrates. EP1042339 and U.S. Pat. No. 6,352,722 describes isomers of derivatives of sucrose, lactose, cellobiose and trehalose for drug delivery. In this patent, the medicinal molecules are incorporated in a carbohydrate matrix to be administered to the patient.

Radiotherapy is able to provide local control of the primary tumor and is not suitable for treating patients with metastatic disease. However, by utilizing the weak immune stimulating effects that radiotherapy provides, in combination with potent immune modulating drugs, it may be possible to cure patients with metastatic disease and obtain systemic tumor control. Cancer immunotherapy attempts to stimulate the immune system to reject and destroy tumors. Radiotherapy (RT) induces tumor cell death by several mechanisms, one represented by induction of immunogenic cell death that leads to secretion of immunogenic proteins like Calreticulin and HMGB1, and small molecules like ATP. These factors activate antigen-presenting cells like monocytes, dendritic cells (DC) and macrophages in the tumor microenvironment. Furthermore, the cells phagocytose dead tumor cells and cell components, and migrate to local lymph nodes to raise an antigen specific response against antigens from the resident dead tumor cells. Unfortunately, radiation alone does not induce a sufficiently high immunogenic response to provide a specific immuno-dependent eradication of the cancer cells due to the immunosuppressive environment, systemically as well as locally in the tumors. An effect caused by M2 macrophages, Treg-cells, immature DCs and myeloid derived suppressor cells. However, a combination of radiotherapy with administered Toll Like Receptor (TLR) agonists or other immune stimulating compounds can potentially provide a sufficiently high immune cell activation to induce a highly effective systemic response.

The combination of radiotherapy with chemotherapeutic drugs or radiosensitizers is also highly interesting for combination therapies if efficient drug delivery systems were available.

One aim of the present invention is to provide new formulations comprising gel-forming, low-viscosity systems that are easy to administer parenterally, and wherein the present invention provides good control of drug release and potentially also visualization by one or multiple imaging modalities. Example 32 demonstrates the flexibility of the system and how the release kinetics from the injectable liquid gel can optimized based on the lipophilicity of the drug, with the most lipophilic drug displaying the slowest release rate and lowest release at the time points evaluated. The release kinetics from the gels may therefore be optimized towards the intended/optimal period of biological activity/stimulation by selecting drugs based on lipophilicity. Rational selection of drugs for multidrug release can therefore be adjusted to achieve release kinetics that are adjusted according to the optimal biological period of stimulation for the individual drugs.

The intricate link between immune cell infiltration and response to conventional cancer therapies has been well established for the majority of cancers. There is therefore an intricate link between immune recognition and immune cell infiltration and prognosis independent of therapeutic intervention. There are four standard treatments of cancer; surgery, chemotherapy, radiotherapy and immunotherapy, which can be combined to provide treatment benefit for patients. As for radiotherapy, several chemotherapeutics induces cancer cell death by mechanisms that include the induction of immunogenic cell death (ICD). ICD stimulate the secretion of immunogenic proteins like Calreticulin and HMGB1, and small molecules like ATP. These factors activate antigen-presenting cells like monocytes, dendritic cells (DC) and macrophages in the tumor microenvironment. These cells phagocytose dead cancer cells and cell components, and present these antigens to immune cells. If suitable antigens are presented and sufficient immune stimulation occurs the immune system may raise a specific response towards the antigens experienced. Unfortunately, radiotherapy and/or chemotherapy are very rarely able to induce a sufficient immunogenic response to induce curative anti-cancer immunoreactivity due to the immunosuppressive environment in the solid tumors. Solid cancers are composed of cancer cells, stromal cells and immune cells and these cells orchestrate the formation of a highly immunosuppressive environment in the solid tumors which hinders the activation of an anti-cancer immune response. An effect caused by immunosuppressive cells, including; M2 macrophages, regulatory T cells, immature DCs, N2 neutrophils and myeloid derived suppressor cells. However, the antigen releasing effects of radiotherapy and chemotherapy may become highly attractive in combination with immune modulating therapy that aims at transforming and modulating the immunosuppressive environment in solid tumors towards and immune active type. The present invention can transform the immunosuppressive tumor microenvironment towards an immune activating type that stimulates, support and polarizes the immune system towards recognizing the cancer cells as foreign and mount a systemic immune response. Adoptive transfer of autologous or allogenic immune cells with reactivity towards e.g. specific cancer antigens are subject to intense research and several has entered clinical evaluation for the treatment of solid malignancies. However, as for the potential immune cells raised endogenously in patients undergoing chemotherapy or radiotherapy, the adoptively transferred cells suffers in the highly immunosuppressive tumor microenvironment. Adoptive cell transfer (ACT) technologies would therefore be significantly potentiated by therapies that could activate the supportive immune system and stimulate the recruitment of transferred cells to the cancerous tissue(s). The possibility to provide a controlled local drug release is also indicated and attractive in a number of clinical conditions and disease, including, but not limited to; inflammatory, infectious, degenerative, non-healing tissue, poor vascularization and autoimmune disease. Across these and other conditions or disease the possibility to provide accurate, image guided delivery of drug delivery systems that provide a controlled release is very attractive. The reasons include, but are not limited to; the possibility to achieve high drug concentration, reduce systemic exposure to antibiotics, achieve antimicrobial activity with reduced risks for developing antimicrobial resistance, combined multidrug activity, local activity that is able to direct revascularization and tissue regeneration and reduce systemic spill over of systemically intolerable drugs or drug combination. The ability of the system to provide sustained release and regional activity and low systemic exposure is demonstrated in example 31. In example 31 the incorporation of radiolabelled drug allowed for the evaluation of retention at the injection site over time and evaluation of blood pharmacokinetics and biodistribution. The data demonstrate that free drugs injected in tumors are directedly washed out and intravenously administered drug achieve very low activity in tumors. The gel technology overcomes the direct washout. The lower systemic spill-over and retention in tumors observed for the radiolabelled drug when incorporated in a gel formulation is optimal for reducing adverse effects and systemic immune activation. The gel sustained release can therefore provide a safe sustained intratumoral stimulation of drugs that poorly tolerated systemically or associated with negative therapeutic effects if distributed systemically. The retention characteristics makes the gel drug delivery optimal for multitargeted immune activating combination therapies without compromising safety. The multidrug combination may include both multiple drugs released from the gel, but also combination with systemically administrated therapies that are not tolerated if all drugs were administered systemically. The observed sustained/long intratumoral stimulation makes it possible to have dosing frequencies/intervals that are attractive for patient compliance and easily adaptable to current standard clinical dosing regimens for available therapies.

The present invention provides a controlled drug delivery system for direct injection in the tissues that are to be immune modulated or stimulated or to provide regional antimicrobial control by the drug included in the drug delivery system. The injected tissue can be primary solid cancers or metastatic lesions, soft tissue or bone tissue where there is inflammation or infectious disease, chronic wounds, diabetic foot ulcer, inappropriate soft tissue or bone healing, vascular disorders, nerve disorders or immune mediated disease. The controlled drug release technology provides an attractive technology for cancer therapy in combination with ICD inducing chemotherapy and radiotherapy and may furthermore support the cell trafficking, survival, recruitment and activity of ACT therapies. The present invention may include both immune stimulating and modulating drugs both also comprise of ICD inducing chemotherapeutics, or a combination of several of these, to achieve intratumoral cancer cell death, antigen release and immune activation. The indications for the controlled drug release technology is furthermore attractive for the treatment of regional or loco-regional diseases or conditions.

The possibility to provide a controlled release of immune modulating therapeutics in a specific region or cancerous lesions provides the opportunity to activate the immune system directly in the tissue of interest to establish an anti-cancer response or depending on the drug or drug combinations included for controlled release also treat diseases or conditions that include; infectious, inflammatory disease, immune mediated disease, vascular disorders or support and stimulate soft tissue, chronic wound, bone, vascular or nerve healing or growth. The invention is demonstrated to provide a controlled release that allows for prolonged stimulation of the plastic and highly reactive immune cell environment in solid tumors and thereby optimally supports the formation of durable anti-cancer response. The invention is therefore also indicated for the treatment of regional or locoregional diseases or conditions that benefit from sustained drug activity. By stimulating an activation of the environment in solid tumors the present disclosure provides the possibility to reactivate the immune system towards actively recognizing the cancer cells to subsequently establish a potent immune response. The present invention provides a controlled release of multiple immune modulating drugs at the injected site(s). In the case of cancer, The injected sites can be primary malignant tumors (intratumoral) or metastases, and in the case of inflammatory, infectious, immune mediated, non healing disease or degenerative disease the injected site can be the specific tissue(s) or region(s) of disease by either single or multiple injections in several sites including multiple repeated dosings. Intratumoral and intrametastatic, as defined by direct injection of immune stimulatory agents into the malignant tumor or metastasis itself, can provide superior priming of the anti-cancer immune response. Furthermore, direct injection into the cancerous tissue or diseased tissue can, not only reduce systemic exposure, off-target toxicities, and the amounts of drug used, but also induce stronger anti-cancer, antimicrobial, regenerative or tissue or immune modulating activity in the injected lesion and in the case of cancer stimulate immune reactivity towards distant non-injected lesions. For cancer therapy A significant amount of clinical and preclinical reports supports the concept that some intratumoral immunotherapeutic strategies may overcome resistance to immune checkpoint inhibitor monotherapies by priming T cells and/or allowing their intratumoral homing function. The effect of immune modulating therapies injected in specific lesions therefore has significant effects far beyond acting locally and thereby provides the possibility to mount the required systemic spillover of the locally raised anti-cancer immune cells to eliminate distant disease and vaccinate cancer bearing subjects against cancer recurrence. Following the possibility to achieve high local drug concentrations the present disclosure can provide improved regional antimicrobial control in subjects with infectious disease. Following the possibility to achieve high local drug concentrations the present disclosure can also provide improved regional therapy for conditions with compromised or inappropriate bone, soft tissue, vascular or nerve healing. Following the possibility to achieve high local drug concentrations the present disclosure can provide a method to support or stimulate the regional or loco regional cell infiltration or stimulate the survival or polarization of systemically or locally injected cell compounds (e.g. stem cell products or adaptive cell transfer products). The present invention provides injectable liquids that solidifies after administration to human or animal tissues after which it provides a system for controlled drug release and/or acts as a tissue marker with imaging capabilities across a range of imaging modalities. Upon injection, the injected liquid solidifies to form a self-coalescing gel-like compound that provides a controlled and sustained release of incorporated immune modulating compounds, cytotoxic chemotherapeutics, anti-inflammatory agents, immunosuppressive agents, protease inhibitors, cell signaling modulating drugs, antibiotics, epigenetic agents, tissue modulating agents, soluble cell signaling agents. The invention provides optional radiographic contrast by the inclusion of iodinated lipids in the formulation. This provides fiducial marker properties and allow for the verification of injections and sites, localization of depot and monitoring and planning possibilities for subsequent administrations. The gel-forming liquid technology has been extensively demonstrated to be feasible in a clinical setting. The possibility to inject using small gauge needle technology and image guidance provides the possibility to accurately inject basically any anatomical location in patients. Example 35 demonstrate how the radiographic contrast of iodinated gels provides the possibility to identify gel depot formation and location. This provides physicians with accurate tracking and validation possibilities when evaluating therapeutic performance. Furthermore, the image contrast can guide injections and positioning of multiple and/or subsequent gel administration in the specific tissue of interest. In the specific example the imaging properties established that contrast is sufficient to identify the gel in both soft tissue and bone. The studies furthermore demonstrated that gel formulation of this viscosity and with these coalescing properties can be form well circumscribed drug depots in both soft tissue and bone using a clinical injection approach. This validates that the gel for soft tissue and bone drug delivery. The gel technology can therefore provide controlled release drug depots in soft tissue and bone, which has clinical indication, including; peri- and intra-osseous and bone inflammatory and infectious disease, soft tissue inflammatory and infectious disease, localized vascular disease and malfunction, mixed soft tissue and bone inflammation and infection, non-healing and non-union disease in soft tissue and bone and immune mediated tissue destructive activity in soft tissue and bone. The technology has been demonstrated to have positional and geometrical stability and thereby secures accurate delivery of drugs throughout the release period. The invention includes the controlled release of multiple drugs in the injected lesion/region. The flexibility of the injectable liquid gel-forming technology provides optimal inclusion of multiple drug for which the release kinetics of the individual drugs may be controlled to achieve the optimal stimulation from a therapeutic point of view.

The flexibility of the gel-forming technology and the therapeutic potential of a multidrug intratumoral release technology is demonstrated observed in example 34. The example illustrates how Immunogenic cell death (ICD) inducing anti-cancer therapies can stimulate cancer associated antigen release, antigen recognition and presentation and how this can be combined with immustimulating therapy. Here, the gel formulation provide fast release and a short stimulation period where ICD of cancer is achieved. The example illustrate how the understanding of how to modulate the release kinetics from the gel makes it possible to overcome these issues. The gel formulation that provide a fast release of ICD inducing chemotherapy (mitoxantrone and doxorubicin) and slow release of immunotherapeutics (R848 and RepSox) display therapeutic efficacy and were well tolerated. This demonstrate that the gel technology can provide biologically optimized and effective drug system in a multidrug and multitargeting gel formulation.

The therapeutic targets of the drugs include; innate immune stimulating drugs (e.g. toll like receptor (TLR) agonists, RIG-1-like receptor agonists and Stimulator of Interferon Receptor (STING) agonists, Nucleotide-binding oligomerization domain-like (NOD-like) receptor agonists), immune activation pathway inhibitors and activators (e.g. Tumor necrosis factor alpha (TNF-α), TNF-a receptor blocking molecules, tumor necrosis factor super family receptor (TNFSFR) agonists (CD40, CD27, CD137, GITR agonists), OX40 (CD124) agonists, T-Cell Immunoreceptor With Ig and ITIM Domains (TIGIT) agonists), transcription factor modulators, immune cell polarizing and immune modulating drugs (e.g. transforming growth factor beta inhibitors (TGF-ßi), T box-containing protein expressed in T cells stimulators, glycogen synthase kinase 3 inhibitors, signal transducer and activator of transcription (STAT) inhibitors, Wnt/β-catenin signaling targeting therapy, Phosphoinositide 3-kinases (PI3Ks) inhibitors, c-KIT inhibitors, mammalian target of rapamycin (mTOR) inhibitors, C-Myc inhibitors, MET inhibitors, BRAF inhibitors, MEK inhibitors, DNA methyltransferase inhibitors (DNMTi), histone deacetylase inhibitors (HDACi), histone methyltransferases inhibitors (HMTi), histone acetyltranferases inhibitors (HATi), histone demethylases inhibitors (HDMi), proteins binding to methylated and acetylated histones inhibitors (PAHi and PMHi)), lymphocyte activating and modulating therapeutics (e.g. programmed death-ligand 1 (PD-L1) inhibitors, programmed death-ligand 2 (PD-L2) inhibitors, programmed cell death protein 1 (PD-1) inhibitors, anti-cytotoxic T-lymphocyte-associated protein-4 (anti-CTLA-4) inhibitors, T-cell immunoglobulin and mucin domain-3 (TIM-3) inhibitors, lymphocyte activating gene 3 (LAG3) inhibitors, Tyrosine phosphatase SHP2 inhibitors), immune metabolism and inflammatory programming therapeutics (e.g. indoleamine 2,3-dioxygenase-1 (IDO1) inhibitors, arginase inhibitors, hypoxia inducible factor 1 (HIF-1) inhibitors, hypoxia inducible factor 2 (HIF-2) inhibitors, cyclooxygenase (COX) 1 and/or 2 inhibitors) and cell death inducing chemotherapeutics, antimicrobial agents including and antibibiotics, inflammation modulating drugs (e.g. inhibitors and activators of interleukin, including; IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36 and IL-37) inhibitors and activators, immunosuppressive agents (e.g. synthetic glycocorticoids, ciclosporine, Azathioprine, ketoconazole), acute phase protein inhibiting drugs (e.g. inhibitors of: C-reactive protein, Serum amyloid P, Serum amyloid A, Complement factors, Mannan-binding lectin, Fibrinogen, prothrombin, Plasminogen activator inhibitor-1 (PAI-1), tissue Plasminogen Activator (tPA), Alpha 2-macroglobulin, Ferritin Ceruloplasmin, Haptoglobin, (Alpha-1-acid glycoprotein, AGP), Alpha 1-antitrypsin, and Alpha 1 antichymotrypsin), drugs modulating extracellular matrix composition (e.g. matrix metalloproteinases (MMP) including (MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28), Osteoconductive and osteoinductive agents (e.g. osteoprotegerin agonists and/or Glycogen synthase kinase 3b inhibitors, transforming growth factor beta-1-3),

The present invention provides a controlled multidrug controlled release system for intra-lesional injection in cancerous tissue(s) or injection into normal or diseased tissue or body cavities or spaces across all anatomical locations using small gauge needle injection technologies. The multidrug delivery system is intended for use as monotherapy or in combination with ICD inducing radiotherapy, chemotherapy and for combination with ACT or stem cell therapy. The system includes the possibility to provide controlled release of both single drugs and multiple drugs. The system can accommodate multiple drug classes and categories and can therefore be optimized chronologically and individually towards the optimal immune stimulation or supression, tissue regeneration support, anti-inflammatory or antimicrobial therapy required in treated subjects throughout their course of therapy.

### SUMMARY OF THE INVENTION

The present invention relates to a composition comprising non-water soluble dissacharides or monosaccharides or trisaccharides and oil, solvent and at least one pharmaceutical ingredients, wherein at least 50% of the non-water soluble dissacharides are carbohydrates selected from Lactose octapropionate, Lactose octaisobutyrate, Sucrose octabenzoate, Methyl hepta-O-isobutyryl-α,β-lactoside, α,β-Lactose octa para-iodobenzoate, 3-iodobenzyl hepta-O-isobutyryl-α,β-lactoside, or mixtures thereof, and wherein the oil is selected from glycerol trihexanoate, Glycerol trioctanoate, Glycerol tridecanoate, Lipiodol, and wherein the composition is a liquid before administration into the human or animal body and increases in viscosity by more than 2,000 centipoise (cP) and more than 100 fold after administration.

### DETAILED DESCRIPTION OF THE INVENTION

The present invention relates to a composition comprising non-water soluble dissacharides and oil, solvent and at least one pharmaceutical ingredient for the treatment of disease in humans or animals.

### Definitions

“Non-water soluble carbohydrates” refers to carbohydrates that are insoluble in water, which is defined as carbohydrates that precipitates when the concentration exceeds 0.1 M at 25 degrees Celsius.

In the context of the present invention, a “gel” is defined as a carrier matrix in which the detectable agent (contrast agent) or active pharmaceutical ingredient is dispersed and/or dissolved within. The term “gel” as used in the present invention includes systems such as gels or amorphous glass matrices, crystalline solids, amormphous solids, which upon injection into a human or an animal increases viscosity where the composition changes from being liquid like to gel like in its appearance.

With the term “hydrophobicity” we refer to the effect that molecule is seemingly repelled from water, that is a molecule that has a log P>0.

With the term “viscosity” we refer to that the viscosity of a fluid is a measure of its resistance to gradual deformation by shear stress or tensile stress

With the term “gel-like” compound or material, as used herein, we refer to any compound comprising some of the properties of a gel i.e. a material that exhibits limited flow when in the steady-state. By weight, gels are mostly liquid, yet they behave like solids due to a three-dimensional interactions within the liquid.

In the present disclosure the compound 6,6′-(2,4,6-triiodophenoxy)acetoxy-isobutyric-sucrose is referred to as “XSAIB”

The terms “drug”, “medicament”, “agent”, “active pharmaceutical ingredient” or “pharmaceutical agent” as used herein include, biologically, physiologically, or pharmacologically active substances that act locally or systemically in the human or animal body.

The terms “treating”, “treatment” and “therapy” as used herein refer equally to curative therapy, prophylactic or preventative therapy and ameliorating therapy. The term includes an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

With the term “Reducing carbohydrate” we refer to a reducing sugar is any sugar that is capable of acting as a reducing agent because it has a free aldehyde group or a free ketone group. All monosaccharides are reducing sugars, along with some disaccharides, some oligosaccharides, and some polysaccharides. The monosaccharides can be divided into two groups: the aldoses, which have an aldehyde group, and the ketoses, which have a ketone group. Ketoses must first tautomerize to aldoses before they can act as reducing sugars. The common dietary monosaccharides galactose, glucose and fructose are all reducing sugars.

With the term “Non-reducing carbohydrates” we refer to Non-reducing disaccharides like sucrose and trehalose have glycosidic bonds between their anomeric carbons and thus cannot convert to an open-chain form with an aldehyde group; they are stuck in the cyclic form. Reducing disaccharides like lactose and maltose have only one of their two anomeric carbons involved in the glycosidic bond, while the other is free and can convert to an open-chain form with an aldehyde group.

With the term “Anomeric centre” and “anomers” we refer to The anomeric centre of a sugar is a stereocentre created from the intramolecular formation of an acetal (or ketal) of a sugar hydroxyl group and an aldehyde (or ketone) group. The two stereoisomers formed from the two possible stereochemistries at the anomeric centre are called anomers.

With the term “monogel” we refer for to any gel that contains one single drug, medicament, agent, pharmaceutical ingredient or pharmaceutical agent as used herein include, biologically, physiologically, or pharmacologically active substances that act locally or systemically in the human or animal body. With the term “combigel” we refer for to any gel that contains two or more drugs, medicaments, agents, or pharmaceutical agents as used herein include, biologically, physiologically, or pharmacologically active substances that act locally or systemically in the human or animal body.

All gel compositions described in the current disclosure are given as weight ratio or weight percent unless otherwise stated.

With the term “TLR7 agonist” we refer to pharmaceuticals that can be a TLR7 agonist or both a TLR7 agonist and a TLR8 agonists.

### DETAILED DESCRIPTION OF THE INVENTION

The formulation is preferably in the form adapted for parenteral administration and/or for administration using topical route, and/or for administration using intracavitary routes such as bladder, uterus, and vagina, and should preferably consist of pharmaceutically acceptable constituents. The formulation that as such has a comparable low viscosity is intended for injection in the body of a human or animal, where after the formulation becomes more viscous, i.e. it goes through a sol-gel transition (liquid to gel) transition, due to the presence of the gel-forming system. It is preferred that the viscosity of the formulation after injection in the body of a human or animal increases by at least 50%, such as at least 80%, such as at least 100%, or at least 150%, or at least 200%, or at least 300%, or at least 500%, or at least 750%, or at least 1000%, or at least 10,000%, or that the formulation becomes essentially solid (non-viscous).

The formulation is preferably adapted for injection via a thin needle used for injection into a body or surgical related procedures, such as but not limited to biopsy. The viscosity of the gel-forming formulation before injection can be any suitable viscosity such that the formulation can be parenterally administered to a patient.

The formulation can also be administered by applying to a wound by a syringe or by smeering into a wound during surgery or surgical procedures. The formulation can also be administered to diseased tissue of a human or animal body where the composition is administered through a hypodermic needle, pig-tail catheter, intravascular catheter, endoscopy aspiration needle, bone marrow aspiration needle and a syringe, an endoscope, a bronchoscope, bone marrow injection device, stereotactic injection frame and preferably under image guidance.

Exemplary formulations include, but are not limited to, those having a viscosity (prior to administration/injection) lower than 10,000 centipoise (cP), e.g. lower than 2,000 cP, such as 10 to 2,000 cP, such as 20 to 1,000 cP, such as 150 to 350 cP, such as 400 to 600 cP, such as 600 to 1,200 cP or such as 1,000 to 2,000 cP, or 10 to 600 cP, or 20 to 350 cP, at 20° C. Alternative formulations include, but are not limited to, those having a viscosity (prior to administration/injection) lower than 10,000 centipoise (cP), e.g. lower than 2,000 cP, such as 10 to 2,000 cP, such as 20 to 1,000 cP, such as 150 to 350 cP, such as 400 to 600 cP, such as 600 to 1,200 cP or such as 1,000 to 2,000 cP, or 10 to 600 cP, or 20 to 350 cP, at 5° C. When referred to herein, the (dynamic) viscosity is measured at the specified temperature in accordance with the method described in ASTM D7483. Gels in the present invention are formed by hydrophobic interactions and/or physical (non-covalent) cross-links by complexation, hydrogen bonding, desolvation, Van der Waals interactions, ionic bonding, combinations thereof, and the like.

The gel forming compositions may be loaded with organic x-ray agents such as iodinated lipid, polymers or sugars for x-ray imaging, or may contain contrast agents for MR imaging, ultrasould imaging, fluorescence imaging.

Pharmaceutical agents can furthermore be covalent or non-covalently embedded in the gel.

### Gel Forming Component

Gel comprising components: The gel solution, comprising solvents, oils (co-solvent), gel-forming carbohydrates ester and drug compounds, is a viscous fluid with viscosities in the range 100-1500 cP. Upon administration of the solution into tissues, the solution is in contact with interstitial fluids which causes non-solvent induced phase separation (NIPS) to occur. In this process, the solvent of the gel solution diffuses into the aqueous phase (interstitial fluids), and the oil (co-solvent) and gel-forming carbohydrate esters forms a high viscosity fluid, solidify or precipitate or a combination thereof forming a hydrophobic depot at the site of injection. The viscous fluid, solid, precipitate or combinations thereof is referred to as a gel or gel depot.

Depending on the physiochemical properties of the solvent, oil (co-solvent), gel-forming carbohydrate ester and drug substance, varying properties can be obtained for the release rate of drug substances, viscosity of the gel solution and of the formed gel.

Solvents of the gel solution: Solvents of the gel solution are soluble in hydrophobic substances such as the oil (co-solvent) and gel forming carbohydrates, as well as in hydrophilic substances such as water. This partial hydrophilic/hydrophobic property of the solvents drives the non-solvent induced phase separation, since the solvents of the gel solution readily diffuses out of the gel solution or gel when exposed to an aqueous environment. Solvent with this amphipathic property have log P values in the range −3.0 to 3.0, such as −2.0 to 2.0, for example −1.5 to 1.5, such as −1.0 to 1.0.

Depending on the log P of the chosen solvent, differing solvent efflux rates and drug burst releases may occur. Solvents with higher log P values interact stronger with the hydrophobic gel depot, and are less soluble in water; hence solvent efflux occurs less rapid.

The chemical composition of the solvent (dispersion medium) should not be particularly limited, and examples include biocompatible organic solvents such as ethanol, ethyl lactate, propylene carbonate, glycofurol, N-methylpyrrolidone, 2-pyrrolidone, propylene glycol, acetone, methyl acetate, ethyl acetate, methyl ethyl ketone, benzyl alcohol, triacetin, dimethylformamide, dimethylsulfoxide, tetrahydrofuran, caprolactam, decylmethylsulfoxide, such as but not limited to N-methyl-2-pyrrolidone, glycofurol, polyethylene glycol (PEG), benzyl benzoate, triglycerides, acetone, benzyl alcohol, V-(betahydromethyl) lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, esters of caprylic and/or capric acids with glycerol or alkylene glycols, oleic acid, peanut oil, polyethylene glycol, propylene carbonate, 2-pyrrolidone, sesame oil, [±]-2,2-dimethyl-1,3-dioxolane-4-methanol, tetrahydrofuran, diethylene glycol monoethyl ether, carbitol, triacetin, triethyl citrate, and combinations thereof; or desirably from trichlorofluoromethane, dichlorofluoromethane, tetrafluoroethane, dimethyl ether, propane, butane, and combinations thereof; or specifically from caprylic/capric triglyceride, oleic acid, 1-dodecylazacycloheptan-2-one and the like. Although the gel formulation can be stably dispersed in these solvents (dispersion media), the solvents may be further added with a saccharide derivatives of for example, triglycerides such as tri-pentanoyl glycerol, tri-octanoyl glycerol, tri-dodecanoyl glycerol, a monosaccharide such as glucose, galactose, mannose, fructose, inositol, ribose and xylose, disaccharide such as lactose, sucrose, cellobiose, trehalose and maltose, trisaccharide such as raffinose and melezitose, and polysaccharide such as α-, β-, or γ-cyclodextrin, sugar alcohol such as erythritol, xylitol, sorbitol, mannitol, and maltitol, or a polyhydric alcohol such as glycerin, diglycerin, polyglycerin, propylene glycol, polypropylene glycol, ethylene glycol, diethylene glycol, triethylene glycol, polyethylene glycol, ethylene glycol mono-alkyl ether, diethylene glycol mono-alkyl ether and 1,3-butylene glycol.

Examples of more preferable solvents are polyhydric alcohol such as glycerin, diglycerin, polyglycerin, propylene glycol, polypropylene glycol, ethylene glycol, diethylene glycol, triethylene glycol, polyethylene glycol, polyethylene glycol (PEG), benzyl benzoate, triglycerides, acetone, benzyl alcohol, ethanol, ethyl lactate, propylene carbonate and Dimethyl Sulfoxide, 1-butanol, 2-butanol, Tert-butylmethyl ether, Ethyl ether, Ethyl formate, Heptane, 3-Methyl-1-butanol, Methylisobutylketone, 2-Methylisobutylketone, 2-Methyl-1-propanol, Pentane, 1-Pentanol, 1-Propanol, 2-Propanol

Examples of most preferable solvents but not limited to are ethanol (EtOH), propylene carbonate (PC), Dimethyl Sulfoxide (DMSO) or Benzyl Alcohol (BA or BnOH)

Solvent effects: The solvents of the gel, e.g. EtOH, DMSO, PC and BnOH, have differing polarity reflected in their Log P values given in example 1. Solvent with the lowest Log P or highest preference for water are release at a higher rate. The release of solvent causes an increase in viscosity of the gel, and for SuBen gels, EtOH displays the fastest increase in viscosity compared to PC and BnOH (Example 30). For LOIB gels, DMSO displays the fastest increase in viscosity followed by EtOH and Acetone. The solvents of the gel thus govern the rate of increase in viscosity, which may be utilized for design of rapidly or slowly settling gels. The solvents also impact the initial viscosity of the gel (prior to injection) to different extend reflecting their capacity to break intermolecular interactions of the gel constituents.

The solvents of the gel may have different reactivity towards transesterification with the API or carbohydrate ester, e.g. alcohols may undergo transesterification with the API or carbohydrate ester. Limited used of nucleophilic solvents such as DMSO, PC or Acetone, ethyl esters or the like can reduce this effect.

Oils of the gel solution (also referred to as co-solvent): Oils of the gel solution are hydrophobic substances that mix poorly with aqueous media. Upon injection of a gel solution into tissue or aqueous media, the oil (co-solvent) and gel forming carbohydrate ester are separated from the solvent due to NIPS. During this phase separation, the oil (co-solvent) carbohydrate ester mixture forms a gel or gel depot with properties governed by the oil (co-solvent) and carbohydrate ester. The co-solvents or oils of this disclosure are characterised by log P values in the range 3 to 10, such as 3 to 7, for example 3 to 5.

Depending on the physicochemical properties of the oil (co-solvent) and carbohydrate ester, and the ratio of these in the gel solution, gel depots with varying viscosities, hydrophobicity and surface tensions may form. This impacts the gel depots interaction with tissues, diffusion of drug substances within the depot and thereby the overall drug release rate.

Examples of oils (co-solvents) are glycerols such as Tripropionin, Tributyrin, glycerol trivalerate, glycerol trihexanoate, glycerol trioctanoate, glycerol tridecanoate, glycerol tridodecanoate, or ethyl hexanoate, ethyl octanoate, ethyl decanoate, ethyl laurate, Ethyl myristate, ethyl palmitate, ethyl stearate, ethyl oleate, and naturally derived oils such as but not limited to corn oil, peanut oil, coconut oil, sesame oil, cinnamon oil, soybean oil, and poppyseed oil, and aliphatic alkyl acyl esters.

More preferred oils (co-solvents) are glycerol trivalerate, glycerol trihexanoate, glycerol trioctanoate, glycerol tridecanoate, ethyl octanoate, ethyl decanoate, ethyl laurate, ethyl oleate, corn oil, peanut oil, sesame oil and poppyseed oil, or Lipiodol.

Most preferred oils (co-solvents, lipid oils) are glycerol trihexanoate (GTH), glycerol trioctanoate (GTO), glycerol tridecanoate (GTD), Lipiodol, ethyl myristate, ethyl pamitate, and ethyl oleoate.

Effect of Lipiodol and Ethyl esters: SuBen gels containing GTO as co-solvent provides sustained release of Resiquimod and Gardiquimod. Prolonged release from SuBen gels can be achieved by replacing GTO with either Lipiodol, Ethyl-palmitate or Ethyl-oleate or mixtures thereof (example 28). The co-solvent therefore has a crucial role for the release of APIs from the formed gel depot.

Co-solvent carbohydrate ester ratio: Similar effect of prolonged release can be achieved by changing the carbohydrate ester co-solvent ratio (Example 3 and 10), i.e. lower co-solvent content results in lower and prolonged release from the gel depot. Reduction of the co-solvent content also affects the viscosity of the final depot (Example 29). The viscosity of the final depot governs the release of API, i.e. increased viscosity slows down diffusion inside the depot and prolongs release of the API. Increased viscosity also stabilizes the final depot towards shearing forces and prevents smearing and migration of the depot. Lowering the GTO content of LOIB gels directly increases the viscosity of the gel depot (examples 29), i.e. a 50% reduction in GTO leads to a 40-fold increase in viscosity. More predominant self-coalescing properties of the gel depot and prolonged release of APIs are expected for gels depots with higher viscosity.

Gel forming carbohydrate esters of the gel solution: Carbohydrate esters comprise the gel forming constituents of the gel solution. Upon solvent efflux caused by NIPS, the carbohydrate esters alone form viscous fluid depots, amorphous solid depots, crystal solid depots or mixtures thereof. The carbohydrate esters are hydrophobic compounds with log P values in the range 3 to 18, such as 3 to 15, for example 3 to 12, such as 4 to 9.

Depending on the hydrophobicity of the carbohydrate ester and incorporation of oils, differing drug substance retention times (release kinetics) can be obtained as well as solubilities of various drug substances may be influenced.

Examples of gel forming carbohydrate esters are carbohydrate ester analogues based on mono-, di-, and tri-saccharides such as but not limited to Glucose (dextrose), Fructose (levulose), Galactose, Sibose, Xylose, such as but not limited to Sucrose, Lactulose, Maltose, Trehalose, Cellobiose, Chitobiose, Isomaltise, such as but not limited to Nigerotriose, Maltotrios, Melezitose, Maltotriulose, Raffinose and Kestose.

The carbohydrates may be fully or partially functionalized/esterified with small organic acids such as but not limited to actetate, propanoic acid, butyrate, isobutyrate, valerate, iso-valerate, benzoic acid or mixtures thereof.

Most preferred carbohydrate esters are Lactose octapropionate (LOP), lactose octaisobutyrate (LOIB), Lactose octabenzoate (LacBen), methoxy-LOIB (meLOIB), Raffinose undecabenzoat (RaBen), Raffinose undecaisobutyrate (ROIB), Sucrose octaisobutyrate (SOIB), Trehalose octaisobutyrate (TOIB) and Sucrose octabenzoate (SuBen).

Anomeric centre and anomers: The anomeric centre of a sugar is a stereocentre created from the intramolecular formation of an acetal (or ketal) of a sugar hydroxyl group and an aldehyde (or ketone) group. The two stereoisomers formed from the two possible stereochemistries at the anomeric centre are called anomers.

Instability of APIs in gels: For the TLR agonists Resiquimod and Gardiquimod, API instability is obtained in LOIB and LOP based gels. In these gels, the primary amine of the Resiquimod and Gardiquimod undergo transesterification with either propionate or isobutyrate (Example 24). The rate of the transesterification reaction is dependent on pH and temperature, and is reduced upon storage of the gel in refrigerator or by addition of acid, such as benzoic acid, isobutyric acid and the like (Example 25). Such features may be used for to limit the transesterification of Resiquimod and the like compounds when formulated in gels.

Effect of anomeric centre: A common feature of LOP and LOIB is their Lactose scaffold that has an anomeric centre. The anomeric centre of Lactose causes higher reactivity towards transesterification. This reactivity is significantly reduced for the isobutyrate esters of the non-reducing carbohydrates, Sucrose, Raffinose and Trehalose. Non-reducing carbohydrate esters are thus less prone to transesterification with the API, solvent or co-solvent. The use of non-reducing carbohydrates thus increases the stability of the carbohydrate esters, and increases the integrity of the gel and the formulated API. Contrary, carbohydrate ester mixtures of anomers show less tendency to crystallization, which improves their solubility in solvents and co-solvents.

Effect of organic acid: Similar reduction in reactivity towards transesterification may be obtained by the selected organic acid used for esterification of the carbohydrate scaffold. Reactivity towards transesterification is found to follow the sequence LOP˜LOIB>LacBen, i.e. benzoate esters display less reactivity towards transesterification (Example 26). Compared to benzoate esters, higher reactivity is expected for acetate, propionate, butyrate or isobutyrate ester derivatives of Sucrose, Raffinose, Trehalose and the like. No detectable transesterification of Resiquimod is detected when formulated in benzoate esters of Lactose (LacBen), Sucrose (SuBen) or Raffinose (RaBen). Overall, low reactivity is expected for all non-reducing and reducing carbohydrate benzoate esters.

Instability in mixed carbohydrate esters: For the mixed acetate isobutyrate ester of sucrose, SAIB, higher degree of transesterification is obtained compared to e.g. sucrose octaisobutyrate (Example 26). This underlines the increased reactivity towards transesterification when acetate esters are present on the scaffold. Consequently, SAIB based formulations of Resiquimod are neither stable at body temperature nor in the refrigerator.

Blocking of the anomeric centre: The reactivity of lactose octaisobutyrate (LOIB) is fully impeded by substitution of the isobutyric acid on the anomeric centre with a methoxy ether (Example 27). In MeLOIB gels, Resiquimod shows no transesterification upon storage at body temperature for extended time periods. Ether protection groups on the position of the anomeric centre abolishes the reactivity and increases the integrity of the gel and the formulated API. Similar decrease in reactivity may be achieved for all reducing carbohydrates via a methoxy ether, ethyl ether or the like for protection on the anomeric centre. Increased stability of non-reducing carbohydrates may also be obtained via ether linked groups.

Quick releasing drug substances: Drug substances that are partially soluble in both hydrophobic and hydrophilic media can be solubilized in the gel solution. Depending on the drug compound it may partition more readily into the aqueous phase. Drug substances with log P less than 0.5, such as less than 0, for examples less that −1 may display large burst release upon injection of the gel solution into buffer or tissue. Drug substances with low log P values (partially hydrophilic compounds) may be stabilized in the gel solution by solvent, oil (co-solvent) and carbohydrate esters with mild hydrophilic properties (lower end log P values).

Examples of quick releasing drug substances are platin-based chemotherapeutics such as oxaliplatin, cisplatin, carboplatin, antimetabolites such as 5-fluorouracil, gemcitabine, cytarabine, capecitabine and methotrexate, anthracyclines such as doxorubicin, daunorubicin, epirubicin, idarubicin, other immunogenic cell death (ICD) inducers such as mitoxantrone and cyclophosphamide.

Slow/sustained releasing drug substances: Drug substances that are partially soluble in both hydrophobic and hydrophilic media can be solubilized in the gel solution. Depending on the hydrophobicity of the drug compound it may interact favourably with the oil (co-solvent) and or gel forming carbohydrate esters and be released slowly from the gel depot. Drug substances that display sustained or slow release have log P values in the range 0.5 to 5, such as 1.0 to 4, for example 1.5 to 3.

Examples of drug substances displaying sustained release are TLR7, agonists such as but not limited to Gardiquimod, Resiquimod and Imiquimod, TGFß-inhibitors such as but not limited to RepSox, Galunisertib and SD-208, GSK inhibitors such as but not limited to CHIR99021, PD0325901, TWS119, SB415286 and GSK-3 inhibitor-X, SHP2 inhibitors such as but not limited to SHP099 and PC-61275, IDO inhibitors such as but not limited to NLG919, and IFN-agonist R08191, RIG-1-like receptor agonists, such as but not limited to KIN1400, KIN1408, Wnt/6-catenin inhibitors such as but not limited to XAV939, drugs with anti-bacterial effect, or an anti-infectious such as but not limited to erythromycin.

Examples of most preferable drug substances displaying sustained release are Resiquimod, RepSox, Galunisertib, TWS119, SB415286, KIN1400, KIN1408, XAV939, and SHP099.

Retained drug substances: Drug substances that are soluble in hydrophobic media can be solubilized in the gel solution. Depending on the hydrophobicity of the drug compound it will interact favourably with the co-solvent and or gel forming carbohydrate esters, which will hinder release of the compound. Drug substances with log P values in the range, log P larger than 5.0 such as larger than 7.0, for example larger than 9.0 display high drug substance retention in the gel depot.

Examples of drug substances that are retained in the gel depot are, lipidated prodrugs, cholesterol derivatives, drug substances linked to hydrophobic carbohydrate esters.

A range of hydrophobic, semi-hydrophobic or amphipathic APIs are soluble in carbohydrate ester gels. Examples of APIs with solubility in the 1-10 mg/g range are TLR agonists (Log P_{Resiquimod}=1.71, Log P_{Imiquimod}=2.65, Log P_{Gardiquimod}=1.40), TGFb inhibitors (Log P_{RepSox}=2.5, Log P_{Galunisertib}=2.64, Log P_{SD-208}=3.5), β-catenin inhibitor (Log P_{XAV939}=2.6) and Rig-1 agonists (log P_{KIN-1400}=6.4). These APIs are all hydrophobic and have Log P values in the range 1.4-6.4.

A solubility of 150 mg/g has been obtained for antibiotics (Log P_{Erythromycin}=2.6), and similar solubility can be expected for APIs with alike hydrophobicity (Log P). TLR agonists, TGFb inhibitors, RIG-1 agonists and others with log P in the range 1.5-6.5 may thus be formulated at concentrations above 10 mg/g such as above 50 mg/g or above 100 mg/g (example 21).

Anthracyclines and the like (Log P_{Doxorubicine}=0.54) can be solubilized in gels in their non-salt (or base) form. Converting the API into to its base form, e.g. by removing a HCl salt, renders the API less polar and more compatible with the hydrophobic gel matrix. Similar increase in solubility can be achieved for all APIs mentioned that are present in an HCl form or the like, using a base washing procedure as exemplified in example 33. Depending on chemical state and Log P (>0.5) of the API, solubilities in carbohydrate ester gels up to 150 mg/g and above can be achieved.

Gels comprising carbohydrate esters such as but not limited to LOIB, SuBen, LacBen or RaBen, co-solvents such as but not limited to GTO, GTH or Ethyl-palmitate and solvents such as but not limited to EtOH, DMSO, PC or BnOH can be produced by simple mixing. Heating and/or sonication may be applied to shorten the time of solubilization, but the time of heating and sonication should be kept minimal in order to minimize degradation or cross reactions of the gel constituents. Once the gel solution is homogeneous and transparent it may be used to formulate APIs. Formulation of APIs is conducted via simple mixing of the API and gel solution e.g. via magnetic stirring. The rate of solubilization may be increased at elevated temperatures, but the time of heating should be kept minimal in order not to minimize degradation or cross reactions of the API with the gel constituents.

### Iodinated Co-Solvents or Oils for Radiographic Visibility

Lipiodol (labeled Ethiodol in the USA), also known as ethiodized oil, is a poppyseed oil used an injectable radio-opaque contrast agent to outline structures during radiological investigations. It is also used in chemoembolization applications as a contrast agent in follow-up imaging.

Lipiodol comprises a combination of iodine and ethyl esters of poppy seed oil. The iodine is intercalated into the constituent fatty acids to produce a mixture of iodostearic and stearic-acid derived esters. Each milliliter contains 480 mg of Iodine organically combined with ethyl esters of fatty acids of poppyseed oil. Lipiodol has a viscosity of 34-70 mPa·s at 20° C., and a density of 1.28 g/cm at 20° C.

Lipiodol is hydrophobic and fully mixable with carbohydrate esters such as but not limited to SAIB, LOIB or SuBen. Upon mixing with SAIB, LOIB or SuBen, Lipiodol offers similar fluidizing properties as the triglyceride co-solvents GTH, GTO and GTD, and lowers the viscosity of the carbohydrate ester:Lipiodol mixture. Upon injection of Carbohydrate-ester:Lipiodol mixtures into water, Lipiodol remain in the carbohydrate ester mixture, due to poor aqueous solubility.

The gel compositions may contain iodine rich compounds for providing radiographic contrast. SuBen and LOIB gel may contain 10-20% CLA-8 corresponding to the iodine content of a gel comprising 15% Lipiodol (Example 22-23). CLA-8 may further be included in ROIB, TOIB, SOIB, RaBen, LacBen gel formulations and the like. Additional CLA-8 like compounds may be fully or partly functionalized carbohydates with one or more aromatic iodine containing acyl group that may contain one or more iodine in different substitution patterns (example 37, FIG. 33). The partly acylated species may have free hydroxyl groups and/or may simultaneously be functionalized with aliphatic or aromatic acyl groups such as acetate, propionate, butyrate, isobutyrate, pivaloate, hexanoate, valerate, isovalerate, benzoate, PABA acylation or PEG acylation in any combination as shown in FIG. 33 (example 37). Different regioisomers (different acylation patterns in the same mixture) and for the reducing sugars different stereo isomers (due to presence of alpha/beta anomeric mixtures) may be present. PABA acylation and PEG acylation as well as free hydroxyl groups provide some hydrophilicity and hence make the resulting carbohydrates more miscible with hydrophilic solvents and drugs. Benzoate, pivaloate and similar hydrophobic acylation provide hydrophobicity making the structures miscible with hydrophobic solvents and active pharmaceutical ingredients.

Moreover, aromatic iodine containing acylation may also be done selectively on primary alcohols or amines in any pattern of full or partial acylation (example 37 FIG. 34). The rest of the positions may be acylated with one or more aliphatic or aromatic acyl groups such as acetate, propionate, butyrate, isobutyrate, pivaloate, hexanoate, valerate, isovalerate, benzoate, PABA acylation or PEG acylation in any pattern. Examples hereof are given in example 37, FIG. 34. Methoxy protection of the anomeric centre of LOIB was additionally explored using iodo-benzyl alcohol. This created CLA-1, an iodinated substitute for LOIB. The CLA-1 formulation (CLA-1:GTO:EtOH 82.5:7.5:10) contains 10% iodine in total, and high CT contrast level is expected for this gel formulation. CLA-1 may further be included in ROIB, TOIB, SOIB, RaBen, LacBen gel formulations and the like.

Additional CLA-1 like compounds may be composed of reducing carbohydrates, where the anomeric centers may be protected as aromatic iodine containing ethers (example 37, FIG. 35) or as aliphatic linear/branched alkyl or aromatic glycosyl ethers (example 37, FIG. 36). The carbohydrates may simultaneously be functionalized with iodine containing aromatic acyl groups at primary alcohols and amines in any acylation pattern. The rest of the positions may be functionalized with or more aliphatic or aromatic acyl groups such as acetate, propionate, butyrate, isobutyrate, pivaloate, valerate, hexanoate, isovalerate, benzoate, PABA acylation or PEG acylation in any acylation pattern. Examples are shown in example 37, FIGS. 35 and 36.

The reducing carbohydrates may also be functionalized as aliphatic linear/branched alkyl or aromatic glycosyl ethers at the anomeric position as in FIG. 36, but without presence of any iodine containing groups (example 37, FIG. 37). The rest of the positions may be functionalized with or more aliphatic or aromatic acyl groups such as acetate, propionate, butyrate, isobutyrate, pivaloate, valerate, hexanoate, isovalerate, benzoate, PABA acylation or PEG acylation in any acylation pattern. Examples are shown in example 37, FIG. 37.

In one embodiment, Lipiodol is mixed with LOIB, SAIB or SuBen and EtOH as solvent. In another embodiment Lipiodol is mixed with LOIB, SAIB or

SuBen and DMSO as solvent. In yet another embodiment Lipiodol is mixed with LOIB, SAIB or SuBen and PC as solvent. In yet another embodiment Lipiodol is mixed with LOIB, SAIB or SuBen and BA as solvent.

In one embodiment, Lipiodol constitute 2.5-50% w/w of the carbohydrate ester solvent mixture, for example 5-30% w/w Lipiodols, such as 7.5-20% w/w Lipiodol, for example 2.5-25% w/w Lipiodol, such as 5-15% w/w Lipiodol.

In one embodiment, LOIB formulations are LOIB:Lipiodol:EtOH (70:20:10), such as LOIB:Lipiodol:EtOH (80:10:10), for example LOIB:Lipiodol:EtOH (75:15:10).

In one embodiment, SAIB formulations are SAIB:Lipiodol:EtOH (82.5:7.5:10), such as SAIB:Lipiodol:EtOH (75:15:10), for example SAIB:Lipiodol:EtOH (60:30:10).

In one embodiment SuBen formulations are SuBen:Lipiodol:BA (60:20:20), such as SuBen:Lipiodol:BA (75:15:10), for example SuBen:Lipiodol:BA (60:25:15).

Upon injection of formulations containing carbohydrate-esters, lipiodol and a solvent such as but not limited to EtOH, PC, BA or DMSO, the gel will set upon solvent efflux forming a depot containing lipiodol and carbohydrate esters. Such depots containing lipiodol contains radiographic contrast and are therefore visible on radiography based imaging modalities, including; fluoroscopy, x-ray and CT imaging. These image modalities may be used for guidance during initial and repeated injections/administrations of the gel or for monitoring the gel depot following injection. Radiopaque gel depots may also be used as liquid fiducials for guiding therapeutic interventions, including, but not limited to; external radiotherapy or as surgical markers.

In one embodiment, gels containing lipiodol has a CT contrast level of 500-10000 HU, such as 500-5000, for example 500-2500, such as 500-1000.

In one embodiment, Lipiodol is formulated as LOIB:Lipiodol:EtOH (75:15:10). In yet another embodiment lipiodol is formulated as SuBen:Lipiodol:EtOH (60:25:15). In one embodiment, Lipiodol is formulated as LOIB:Lipiodol:EtOH (75:15:10) and has an average CT contrast of 1700 HU, in yet another embodiment, lipiodol is formulated as SuBen:Lipiodol:EtOH (60:25:15) and has an average CT contrast of 3000HU.

Gel formulation containing lipiodol as oil (co-solvent) may act as sustained release depots of drug substances, such as, but not limited to; toll like receptor (TLR) agonists, RIG-1-like receptor agonists and Stimulator of Interferon Receptor (STING) agonists, Nucleotide-binding oligomerization domain-like (NOD-like) receptor agonists), immune activation pathway inhibitors and activators (e.g. Tumor necrosis factor alpha (TNF-α), TNF-α receptor blocking molecules, OX40 (CD124) agonists, T-Cell Immunoreceptor With Ig and ITIM Domains (TIGIT) agonists), Tumor necrosis factor receptor super family (TNFRSF) agonists (e.g. OX40 (CD134) agonists, CD40 agonists, 4-1BB (CD137) agonists, CD27 agonists, Glucocorticoid-induced tumor necrosis factor receptor-related protein (GITR/CD357) agonists), inducible T-cell costimulator (ICOS) agonists, transcription factor modulators, immune cell polarizing and immune modulating drugs (e.g. transforming growth factor beta inhibitors and receptor inhibitors (TGF-ßi), T box-containing protein expressed in T cells stimulators, glycogen synthase kinase 3 inhibitors, signal transducer and activator of transcription (STAT) inhibitors, Wnt/6-catenin signaling targeting therapy, Phosphoinositide 3-kinases (PI3Ks) inhibitors, c-KIT inhibitors, mammalian target of rapamycin (mTOR) inhibitors, C-Myc inhibitors, MET inhibitors, BRAF inhibitors, MEK inhibitors, DNA methyltransferase inhibitors (DNMTi), histone deacetylase inhibitors (HDACi), histone methyltransferases inhibitors (HMTi), histone acetyltranferases inhibitors (HATi), histone demethylases inhibitors (HDMi), proteins binding to methylated and acetylated histones inhibitors (PAHi and PMHi)), lymphocyte activating and modulating therapeutics (e.g. programmed death-ligand 1 (PD-L1) inhibitors, programmed death-ligand 2 (PD-L2) inhibitors, programmed cell death protein 1 (PD-1) inhibitors, anti-cytotoxic T-lymphocyte-associated protein-4 (anti-CTLA-4) inhibitors, T-cell immunoglobulin and mucin domain-3 (TIM-3) inhibitors, lymphocyte activating gene 3 (LAG3) inhibitors, Tyrosine phosphatase SHP2 inhibitors), immune metabolism and inflammatory programming therapeutics (e.g. indoleamine 2,3-dioxygenase-1 (IDO1) inhibitors, arginase inhibitors, hypoxia inducible factor 1 (HIF-1) inhibitors, hypoxia inducible factor 2 (HIF-2) inhibitors, cyclooxygenase (COX) 1 and/or 2 inhibitors) and cell death inducing chemotherapeutics, antimicrobial agents including antibibiotics, inflammation modulating drugs (e.g. inhibitors or agonists of interleukin, including; IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36 and IL-37), immunosuppressive agents (e.g. synthetic glycocorticoids, ciclosporine, Azathioprine, ketoconazole), acute phase protein inhibiting drugs (e.g. inhibitors of: C-reactive protein, Serum amyloid P, Serum amyloid A, Complement factors, Mannan-binding lectin, Fibrinogen, prothrombin, Plasminogen activator inhibitor-1 (PAI-1), tissue Plasminogen Activator (tPA), Alpha 2-macroglobulin, Ferritin, Ceruloplasmin, Haptoglobin, (Alpha-1-acid glycoprotein, AGP), Alpha 1-antitrypsin, and Alpha 1antichymotrypsin), drugs modulating extracellular matrix composition (e.g. matrix metalloproteinases (MMP) including (MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28), Osteoconductive and osteoinductive agents (e.g. osteoprotegerin agonists and/or Glycogen synthase kinase 3b inhibitors, transforming growth factor beta-1-3),

Gel formulations containing lipiodol as oil (co-solvent) may act as sustained release depots of one, two or multiple drug substances.

Gel formulations containing lipiodol as oil (co-solvent) and drug substances may be used as fiducial markers for guiding therapeutic interventions, including, but not limited to; external beam radiotherapy or surgical procedures.

In one specific embodiment of the invention the hydration sensitive gel forming component is hydrophobic saccharides and an oil selected from Lactose octapropionate, Lactose octaisobutyrate, Sucrose octabenzoate, lactose octabenzoate, rabinose benzoate or mixtures thereof, glycerol trihexanoate, Glycerol trioctanoate, Glycerol tridecanoate, Lipiodol, ethyl myristate, ethyl palmitate, ethyl oleoate.

The composition of the solvent (dispersion medium) should not be particularly limited, and examples include biocompatible organic solvents such as ethanol, ethyl lactate, propylene carbonate, glycofurol, N-methylpyrrolidone, 2-pyrrolidone, propylene glycol, acetone, methyl acetate, ethyl acetate, methyl ethyl ketone, benzyl alcohol, triacetin, dimethylformamide, dimethylsulfoxide, tetrahydrofuran, caprolactam, decylmethylsulfoxide, such as but not limited to N-methyl-2-pyrrolidone, glycofurol, polyethylene glycol (PEG), benzyl benzoate, triglycerides, acetone, benzyl alcohol, N-(betahydromethyl) lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, esters of caprylic and/or capric acids with glycerol or alkylene glycols, oleic acid, peanut oil, polyethylene glycol, propylene carbonate, 2-pyrrolidone, sesame oil, [±]-2,2-dimethyl-1,3-dioxolane-4-methanol, tetrahydrofuran, diethylene glycol monoethyl ether, carbitol, triacetin, triethyl citrate, and combinations thereof; or desirably from trichlorofluoromethane, dichlorofluoromethane, tetrafluoroethane (R-134a), dimethyl ether, propane, butane, and combinations thereof; or specifically from caprylic/capric triglyceride, oleic acid, 1-dodecylazacycloheptan-2-one and the like. Although the formulation can be stably dispersed in these solvents (dispersion media).

The oils are for example, triglycerides such as tri-pentanoyl glycerol, glycerol trihexanoate, glycerol trioctanoate, glycerol tridecanoate, ethyl octanoate, ethyl decanoate, ethyl laurate, ethyl oleate, corn oil, peanut oil, sesame oil, poppyseed oil, and Lipiodol.

Detergents could be added such as Tween 20 Tween 80, Triton X-100, sodium dodecyl sulfate (SDS), Brij, phospholipids, lysophospholids, sterols and the like;

### Contrast Agents

Contrast may be achieved using organic x-ray contrast agents, such as radiopague agents such as iodinated compounds, which may be combined with chelators of MRI agents such as gadolinium. Chelators may be DOTA, EDTA, or DTPA and chelators will be non-covalently embedded or covalently conjugated to the gel-forming components.

In one embodiment, the gel-forming components further comprise one or more fluorophore compounds for near infrared fluorescence imaging. Said fluorescent dye molecules. Common classes of fluorescent dyes include xanthenes such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes, polymethine, oxabenzanthrane, xanthene, pyrylium, carbostyl, perylene, acridone, quinacridone, rubrene, anthracene, coronene, phenanthrecene, pyrene, butadiene, stilbene, lanthanide metal chelate complexes, rare-earth metal chelate complexes, and derivatives of such dyes. Typical fluorescein dyes include 5-carboxyfluorescein, fluorescein-5-isothiocyanate and 6-carboxyfluorescein; examples of other fluorescein dyes can be found, for example, in U.S. Pat. Nos. 6,008,379, 5,750,409, 5,066,580, and 4,439,356. The species may also include a rhodamine dye, such as, for example, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED), and other rhodamine dyes. The species may alternatively include a cyanine dye, such as, for example, Cy3, Cy3B, Cy3.5, Cy5, Cy5.5, Cy. Or IRDye 800CW, IRDye 680LT, Qdot 800 nanocrystal, Qdot 705 nanocrystal or porphyrazine compounds

In another embodiment, contrast in achieved using small organic iodine containing compounds. Said small organic iodine containing compounds includes commercial available iodinated contrast agents such as diatrizoate (marketed e.g. under the trade name Gastrografen™), ionic dimers such as ioxaglate (marketed e.g. under the trade name Hexabrix™), nonionic monomers such as iohexol (marketed e.g. under the trade name Omnipaque™), iopamidol (marketed e.g. under the trade name Isovue™) iomeprol (marketed e.g. under the trade name Iomeron™) and the non-ionic dimer iodixanol (marketed under the trade name and Visipaque™). Additional examples of small organic iodine containing compounds includes lipiodol and the ones disclosed in WO2009/071605, EP1186305, EP686046, EP108638, EP0049745, EP0023992, WO2003080554, WO2000026179, WO1997000240, WO9208691, U.S. Pat. Nos. 3,804,892, 4,239,747, 3,763,226, U.S. Pat. Nos. 3,763,227 and 3,678,152, but not limited to those.

In one embodiment, the composition according to the present invention is administered using topical route.

In one embodiment, the composition according to the present invention is intra-cavitary administration into existing or established body cavities. The existing cavities include, but are not limited to; urinary bladder, uterus, gall bladder, sinuses, middle ear. The established or formed cavities include, but are not limited to cavities formed in relation to surgery and infections.

### Viscosity of the Formulation

The viscosity of the formulation is before the injection preferably lower than 10,000 cP, in particular lower than 5,000 cP, at 20° C.

Alternatively, the viscosity of the formulation is before the injection typically lower than 3,000 cP at 5° C.

In one embodiment, the gel-forming system of the formulation is preferably one which, after injection or under conditions mimicking those in a human body, forms a gel having a viscosity at 37° C. in the range of 2,000 to 50,000,000 cP. More particularly, the viscosity of the hydrogel can be about 2,000 cP, about 5,000 cP, about 10,000 cP, about 20,000 cP, about 30,000 cP, about 50,000 cP, about 75,000 cP, about 100,000 cP, about 125,000 cP, about 150,000 cP, about 200,000 cP, about 30,000 cP, about 800,000 cP, about 1,000,000 cP, about 2,000,000 cP, about 5,000,000 cP, about 10,000,000 cP, about 20,000,000 cP, about 30,000,000 cP, about 40,000,000 cP, about 50,000,000 cP, or ranges thereof. Preferably, the viscosity of the hydrogel after injection (i.e. when present in the desired location) is above 20,000 cP, e.g. in the range of 20,000 cP to 1,000,000 cP. In particular, the formulation after injection is preferably essentially solid.

### Preferred Properties of the Gel-Forming System

In one embodiment of the invention the composition comprising a non-water soluble carbohydrate, wherein the composition is a liquid before administration into the human or animal body and increases in viscosity by more than 1,000 centipoise (cP) after administration. In one embodiment of the invention the composition comprising a non-water soluble carbohydrate, wherein the composition is a liquid before administration into the human or animal body and increases in viscosity by more than 10,000 centipoise (cP) after administration.

In one embodiment, at least 60% of an administrated amount of the non-water soluble carbohydrate remains more than 24 hours within 10 cm from an injection point when administrated to a human or animal body.

In one preferred embodiment, the mixing of different acylated disaccharides, results in controlled drug release providing tuning of release kinetics for the individual drug. The composition according to the present invention also relates to the release of one or more active pharmaceutical ingredients being controlled by mixing carbohydrates with different hydrophobicity by alteration of the substitutions on the carbohydrate hydroxyl groups. With the aid of tuning the hydrophobicity, the release rate of the present invention may be changed, this implies therefore increased control of the process. Rendering it suitable for controlled release of for example pharmaceuticals and other substances. Active pharmaceuticals may be formulated in various forms and the present invention is to be seen as incorporating various forms of formulations of the active ingredient.

### Other Constituents of the Formulation

In one embodiment a polymer may be used to work as a stabilizer between gel and biological surrounding and therefore, the composition may also comprises a molecule that increase gel stability in the human or animal body, such as an amphiphilic molecule, such as an emulsifier. Therefore in one embodiment the composition comprises poly(ethylene glycol-b-caprolactone) (PEG-PCL), sucrose acetate isobutyrate (SAIB), poly(D,L-lactic acid) (PLA), or poly(lactic-co-glycolic acid) (PGLA), or a combination thereof. In one embodiment of the present invention poly(D,L-lactic acid) (PLA) is added to the non-water soluble carbohydrate causing a reduction of burst release of said encapsulated contents e.g. drugs, particles, contrast agents, etc. The formulation may further include other constituents, such as α-, β-, and/or γ-cyclodextrins and any derivate hereof. Such constituents may form guest/host complexes with the gel forming system and the nano-sized particles, thus, both aiding in the gel formation and possible alter the particle leakage profile [Adv. Drug Delivery Rev., 2008, 60, 1000-1017]. In one very interesting embodiment the gel forming system is based on PEG-PHB-PEG triblock copolymers, α-cyclodextrin and PEG coated solid nano sized particles. In such a formulation, α-cyclodextrin may form inclusion complexes with both the PEG blocks of the PEG-PHB-PEG triblock copolymers and the PEG coated solid nano sized particles which, combined with hydrophobic interactions between the PHB middle block, forms a strong hydrogel with enhanced retention of solid nano sized particles due α-cyclodextrin interactions which thus altering the particle leakage profile.

The formulation may further comprise compounds or polymers, which are visible in imaging modalities other than X-ray imaging.

### Pharmaceutical Agent

The gel-forming formulation may further comprise pharmaceutical agents including prodrugs (in short “drugs”; broadly interpreted as agents which are able to modulate the biological processes of a mammal). These drugs can be formulated as a single drug or as a combination of two or more of the below mentioned drugs in its active form or as a prodrug.

In one embodiment, the active pharmaceutical ingredient is an innate immune activating compound which is a ligand for intracellular proteins and/or receptors; or a ligand for cell surface proteins and/or receptors. The compounds can include the following drug as single therapeutic agents or as combinations of; immune activating compounds, including; Toll-like-receptor (TLR) family; TLR1, TLR2 TLR3, TLR4, TLR5, TLR6, TLR7, TLR8, TLR9, TLR10, TLR11, TLR12, TLR13. Examples of TLR agonists includes; polyinosinic:polycytidylic acid (polyI:C), Polyadenylic-polyuridylic acid (poly A:U), poly I:C-poly-L-lysine (poly-ICLC), poly-ICR, 3p′dsRNA, 3p′dsDNA, 2p′dsRNA, 2p′dsDNA, p′dsRNA, p′dsDNA, dsRNA, dsDNA, ssDNA, ssRNA, Imiquimod (R837), Resiquimod (R848), TMX-10, TMX-201, TMX-202, DSR6434, Gardiquimod, R850, R851, 852A, Isatoribine, S-2761 0, 3M-002 (CL075), 3M-003, 3M-005, 3M-006, 3M-007, 3M-012, 3M-13, 3M-031, 3M-854, CL075, CL097, CL264, IC-31, Loxoribine and other imidazoquinolines, ssPolyU, ANA975, SM360320, SM3244405, RWJ 21757, R 1354, single stranded or double stranded RNA, ORN 02 (5′-UUAUUAUUAUUAUUAUUAUU-3′), ORN 06 5′-UUGUUGUUGUUGUUGUUGUU-3′, CpG-ODN DSLIM, AVE 0675, CpG B oligodeoxynucleotide, 1018, AZD 1419, ODN 1982, CpG B ODN 2006, IMO 2125, CpG A ODN 221 6, CpG A ODN 2336, CpG 2395, CpG ODN 7909, CpG 10101, CpG ODN AVE0675, CpG ODN HYB2093, CpG ODN HYB2055, CpG-ODN IMO-21 25, CpG C ODN M362, Tolamba (Amb a 1 ragweed allergen with covalently linked CpG B class ODN 10 18), Heplisav, 10 181SS, IM02055, IRS954, (flagellin, muramyl dipeptide, saponins such as QS21, Leishmania elongation factor, SB-AS4, threonyl-muramyl dipeptide, L 18-MDP, mifamurtid, A83-01, A4476, GW788383, LY364947, R26871 2, RepSox, SB431542, SB505124, SB525334, SD208, FAK inhibitor 14, PF431 396, PF573228, Y 11 and OM-174, nickel and the like, but not limited to those.

Inhibitors of Toll-like-receptors (TLR) include; AT791, E6446, COV08-0064, COV08-0055 and COV08-0064 and the like, but not limited to those.

STING agonists; compounds include; ADU-S100, C11, Cridanimod, MK-1454, PO-424, H-151, C-176, diABZI compound 3 (2138299-34-8 and 2138299-33-7), diABZI compound 2 (2138300-40-8), 3′3′-cGAMP, 2′3′-cGAMP, ChX0306710, ML RR-S2 CDA ammonium salt, ML RR-S2 CDA, sodium Cridanimod, G10 and the like, but not limited to those.

STING inhibitors, compounds include; H-151, C-176, C-178, STING inhibitor 18, Astin C and the like, but not limited to those. RIG-1-like receptor agonists, examples include; KIN1148, KIN131A, KIN126X, KIN150X, KIN1000, KIN1408, SLR14, MK4621, RGT100, KIN1400 and the like, but not limited to those.

In yet another embodiment the active pharmaceutical ingredient is an immune activating/modulating drug which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include; Tumor necrosis factor alpha (TNF-a) agonists, TNF-a receptor blocking molecules, Interferon (IFN) agonists, examples include; R08191, R08181 and the like, but not limited to those. T-Cell Immunoreceptor with Ig and ITIM Domains (TIGIT) agonists.

In yet another embodiment the active pharmaceutical ingredient is a transcription factor modulator, post translational modying enzyme modulator, immune cell polarizing and immune modulating drug which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system.

The compounds include, as single agents or in combination: including transforming growth factor beta signaling inhibitors (TGF-ß inhibitors) and transforming growth factor beta receptor inhibitors (TGF-ß receptor inhibitors) and including ALK signaling inhibitors and Smad singnaling inhibitors; RepSox, Galunisertib (LY2157299), LY550410, LY580276, TEW-7197, SB 505124, SB 431542, A 83-01, SD 208, LY 364947, SB 525334, SB 505124, D 4476, GW 788388, R 268712, IN 1130, SM 16, A 77-01, SB 431542, LY 364947, R268712, ITD 1, SIS3, LY2109761, LY 3200882, Pirfenidone, LDN-193189, LDN-193189 HCL, K02288, LDN-214117, TEW-7179, DMH1, LDN-212854, ML347, sotirimod, Kartogenin, Hespertin, Alantolacton, Suramin sodium, BML-275 dihydrochloride, BML-275, ALK2-IN-1, Vactosertib, LSKL, Inhibitor of Thrombospondin (TSP-1), SJ000291942, K02288, LDN-212854, ML347, SM 16, TGFβRI-IN-1 and the like, but not limited to those.

The compounds include, as single agents or in combination: including tyrosine phosphatase SHP2 inhibitors; SHP099, PC61275 and the like, but not limited to those.

The compounds include, as single agents or in combination: including Glycogen Synthase Kinase 3 (GSK-3) inhibitors; SB415286, Lithium, Valproic acid, Iodotubercidin, Naproxen, Cromolyn, Famotidin, Curcumin, Olanzapine, Pyrimidine derivater, ARA014418, CHIR 99021, CHIR 99021 trihydrochloride, SB 216763, BIO, Kenpaullone, 10Z-Hymenialdisine, SB 415286, Indirubin, Indirubin-3prime-monoxime-5-sulphonic Acid, Indirubin-3′-oxime, 5-Iodo-indirubin-3prime-monoxime, Indirubin-5-sulfonic acid sodium salt, NSC 693868, TWS 119, TWS 119 ditrifluoroacetate, TCS 2002, MeBIO, BIO, BIO-acetoxime, Bisindolylmaleimide I, Bisindolylmaleimide I hydrochloride, 3F8, TCS 21311, TCS2002, TC-G 24, A 1070722, Lithium Carbonate, TDZD 8, AlsterPaullone, CHIR 99021, CHIR 99021 trihydrochloride, CHIR 98014, tideglusib, AZD2858, AZD1080, LY2090314, 2-D08, IM-12, 1-Azakenpaullone, Bikinin, L807mts, Staurosporine, KT5720, GSK-3 inhibitor IX, Ro 31-8220, CID 755673, GSK-3 Inhibitor XVI, 10Z-Hymenialdisine, GSK-3beta Inhibitor VI, Manzamine A, GSK-3 Inhibitor X, GSK-3 Inhibitor XV, GSK-3beta Inhibitor I, GSK-3beta Inhibitor VII, GSK-3beta Inhibitor II, GSK-3beta Inhibitor VIII, Hymenidin, Bisindolylmaleimide X hydrochloride, 3F8, Isogranulatimide, CR8, L779,450, Aloisine A, GSK-3beta Inhibitor XI, Ro-31-8220, Ro 31-8220 methanesulfonate, Enzastaurin, PHA 767491 hydrochloride, AR-AO 14418-d3, Indole-3-acetamide, Hymenialdisine Analogue 1, CP21R7, Necrosulfonamide, IM12, Leucettine, LY-2090314, Tideglusib and the like, but not limited to those.

The compounds include, as single agents or in combination: including signal transducer and activator of transcription (STAT) inhibitors; Stattic, Cucurbitacin I, Niclosamide, NSC 74859, SD 1008, Cryptotanshinone, Napabucasin, Galiellalactone, S31-201, Nifuroxazide, SH-4-54, AS1517499, Artesunate, BP-1-102, SH5-07, STA-21, HJC0152, APTSTAT3-9R, C188-9-HO-3867, RSVA 405, and the like, but not limited to those.

The compounds include, as single agents or in combination: including Wnt/β-catenin signaling targeting therapy; WAY-316606, IWP, IWP-L6, LGK974, WNT-059, ETC-159, Ant1.4Br/Ant1.4CI, (hetero)arylpyrimidines, Niclosamide, apicularen, bafilomycin, XAV939, IWR, G007-LK, G244-LM, IQ1, pyrvinium, QS11, NSC668036, SB-216763, CHIR99021, BIO(6-bromoindirubin-3′-oxime), LY2090314, DCA, 2-amino-4-[3,4-(methylenedioxy)benzyl-amino]-6-(3-methoxyphenyl)pyrimidine, 2,4-diamino-quinazoline, Quercetin, ICG-001, PKF115-584, BC2059, Shizokaol D, 3289-8625, J01-017a, Derricin, Derricidin, Carnosic acid, Windorphen, TMEM88, KY-02061, KY-02327, BMD4702, DK-520, Sulindac, ICG-001, PNU-74654, E7449 and the like, but not limited to those.

The compounds include, as single agents or in combination: including Phosphoinositide 3-kinases (PI3Ks) inhibitors; Wortmannin, LY294002, PX-866, XL-147, SF1126, GDC0941, PI-103, NCPBEZ235, XL765, GSK2126458, PKI-587, MK2206, PF-04691502, 10187114, CAL-101, Rapamycin, Torinl, AZD-8055, OSI-027, GDC-0032, NVP-BKM120, ZSTK474, BAY 80-6946, BYL719, GDC0326, SAR260301, GDC0980, GNE-317, GNE-477, PF-0491502, PKI-179, PKI-587, INK-1117, CH5132799, AZD8186, IPI-145 Buparlisib, Idelasilib, IPI-549, Pictillisib and the like, but not limited to those.

The compounds include, as single agents or in combination: including tyrosine kinase receptor inhibitors, including but not limited to c-KIT (SCFR), PDGFR, FGFR, VEGFR, and FA; Axitinib, Dasatinib, TKI-258, ST1571, AMG-706, GW786034 HCL, Sunitinib malate, AB1010, PTK787, XL184, BMS-907351, AV-951, OSI-930, MP-470, Ki8751, Telatinib, Pazopanib, TKI-258, CHIR-258, Thyrpostin AG 1296, PKC-412, ISCK03, AP 24534, KRN633, SU6668, Sorafinib, ABT-869, Divitinib, Pazopanib, 4,4prime-Bis(4-aminophenoxy)biphenyl, ISCK03, Tandutinib, SU1652, AGL 2043, PLX9486, BLU-285, AZD2932, PLX3397, MGCD516 and the like, but not limited to those.

The compounds include, as single agents or in combination: including tyrosine kinase receptor agonists, including but not limited to c-KIT (SCFR), PDGFR, FGFR, VEGFR, and FA; DRM/gremlin, and the like, but not limited to those.

The compounds include, as single agents or in combination: including Bruton's Tyrosine Kinase (BTK) and Interleukin-2-Inducible Kinase (ITK) Inhibitors: Ibrutinib, Acalabrutinib and the like, but not limited to those.

The compounds include, as single agents or in combination: including Hedgehog pathway modulators, including Smoothened (Smo), Sonic hedgehog pro-tein (Shh), and Gli1 inhibitors and agonists: SAG, compound 10c, Mercaptobenzoimidazole, cyclopamine, HhAntag, IP1926, GDC-0449, Cur61414, GANT61, IPI-269609 BMS-833923, PF-04449913, HPI1, HP12, HPI3, HP14, JK-184, NMDA298-1, robotnikinin, Purmorphamine, 22(S)-hydroxycholesterol, 20(S)-hydroxycholesterol, GANT58, GANT61 and the like, but not limited to those.

The compounds include, as single agents or in combination: including Mammalian target of rapamycin (mTOR) inhibitors; Temsirolimus, Everolimus, Ridaforolimus (AP23573 and MK-8669), Dactolisib, Omipalisib, Niclosamide and the like, but not limited to those.

The compounds include, as single agents or in combination: including C-Myc inhibitors; JQ1, I-BET151, 10058-F4, 10074-G5, 7594-0035, KJ Pyr 9, ML327, Mycro3, IZCZ-3, KSI-3716, 403811-55-2, Apto-253 and the like, but not limited to those.

The compounds include, as single agents or in combination: including c-Met inhibitors; PHA665752, INC280, SU 11274, AMG208, Golvatinib, PF 02341066, LY 2801653, ARQ 197, PF 04217903, Fortinib, Crizotinib, PHA-665752, SAR125844, Pulsatilla saponin D, SGX-523, BMS-777607, JNJ-38877605, MGCD-265, INCB28060, BMS-794833, BMS-754807, AMG-208, MK-208, MK-2461, E7050, AMG-458, NVP-BVU972, EMD 1214063, AMG-337, LY12801653, S49076, Norcantharidin, NPS-1034, AZD6094 and the like, but not limited to those.

The compounds include, as single agents or in combination: including BRAF inhibitors; BMS-9086662, LGX818, PLX3603, RAF265, R05185426, GSK2118436, PLX4032, Sorafenib, PLX-4720, GDC-0879, AZ304, PLX-8394, LXH254, Dabrafenib mesylate, RAF265, AZ 628, NVP-BHG712, SB590885, ZM 336372, GW5074, TAK-632, CEP-32496, LGX818, BAW2881, CCT196969, RAF709, BGB-283, PLX7904, LY3009120, R05126766, MLN2480, Regorafenib and the like, but not limited to those.

The compounds include, as single agents or in combination: including MEK inhibitors; BIX02188, PD0325901, U0126-ETON, GSK1120212, AZD6244, PD0325901, CI-1040, PD98059, AS-703026, TAK-733, AZD8330, MEK162, PD318088, Honokoil, SL-327, RDEA119, Myricetin, BI-847325, GDC-0973, GDC-0623, APS-2-79 and the like, but not limited to those.

In yet another embodiment the active pharmaceutical ingredient is an epigenetic modulating drug, which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including DNA methyltransferase inhibitors (DNMTi), histone deacetylase inhibitors (HDACi), histone methyltransferases inhibitors (HMTi), histone acetyltranferases inhibitors (HATi), histone demethylases inhibitors (HDMi), proteins binding to methylated and acetylated histones inhibitors (PAHi and PMHi); decitabine, azacitidine, EGCG, zebularine, hydralazine, procainamide, Vorinostat, givinostat, panobinostat, TSA, belinostat, entinostat, CG-1521, romidepsin, ITF-A, ITF-B, valproic acid, OSU-HDAC-44, HC-toxin, magnesium valproate, plitidepsin, tasquinimod, sodium butyrate, mocetinostat, carbamazepine, SB939, CHR-2845, CHR-3996, JNJ-26481585, sodium phenylbutyrate, pivanex, resveratrol, abexinostat, resminostat, dacinostat, droxinostat, Pargyline, clorgyline, bizine, GSK2879552, GSK-J4, KDM5-C70, JIB-04, tranylcypromine, EPZ-6438, GSK126, CPI360, DZNep, GSK343, Ell, BIX-01294, UNC0638, EPZ004777, UNCO224, JQ1, CPI203, RVX-208, I-BET151, I-BET762, i-BET-726, UNC669, UNC1215 and the like, but not limited to those.

In yet another embodiment the active pharmaceutical ingredient is a lymphocyte activating and modulating drugs which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: Programmed death-ligand 1 and 2 (PD-L1 and PD-L2) inhibitors, programmed cell death protein 1 (PD1) inhibitors and PD-L1/PD-L1 checkpoint inhibitors; BMS-8, BMS-37, BMS-57, BMS-71, BMS-105, BMS-202, BMS-230, BMS-242, BMS-1001, BMS-1166, BMS-1165, BMS-2007, BMS-1016, BMS-40210, BMS-8, CA-170, CA-327, SB415286, INCB086550, INCMGA00012, CX072, CCX4503, MAX-10129, vorinostat, panobinostat, azacitidine, decitabine, entitostat, JQ1, I-BET151, GSK503, WO2015/034820, WO2015/033301 and the like, but not limited to those.

The compounds include, as single agents or in combination: including Anti-cytotoxic T-lymphocyte-associated protein-4 (anti-CTLA-4) inhibitors/modulators; Compounds “8 and 9”, ACY-241 and the like, but not limited to those.

The compounds include, as single agents or in combination: including T-cell immunoglobulin and mucin domain-3 (TIM-3) inhibitors; TSR-022, Sym023, ATIK2a, CA-327 and the like, but not limited to those. The compounds include, as single agents or in combination: including Lymphocyte activating gene 3 (LAG3) inhibitors; IMP32, BMS986016 and the like, but not limited to those.

The compounds include, as single agents or in combination: including OX40 (CD124) activators/modulators, examples include; DB36, DB71, DB15, CVN, MGCD0103, SNDX-275 and the like, but not limited to those. Tumor necrosis factor receptor super family (TNFRSF) agonist (e.g. OX40 (CD124) agonists, CD40 agonists, CD27 agonists, 4-1BB (CD137) agonists, Glucocorticoid-induced tumor necrosis factor receptor-related protein (GITR, CD357) agonists, inducible T-cell costimulator (ICOS) agonists and tumor necrosis factor related apoptosis-inducing ligand (TRAIL, CD253, TNFSF10) receptor agonist, examples include; Acrp30-CD40L, DB36, DB71, DB15, CVN, MGCD0103, SNDX-275, dulanermin, and the like, but not limited to those.

In yet another embodiment the active pharmaceutical ingredient is an immunemodulating and/or inflammation modifying enzyme inhibitor or activator, a cellular receptor of metabolites, which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including Indoleamine 2,3-dioxygenase-1 (IDO1) inhibitors; methyl-tryptophan, D-1 MT, L-1MT, tryptophan, epacadostat, GDC-0919, Indoximod, EOS-200271, NLG919, BMS-986205 and the like, but not limited to those.

The compounds include, as single agents or in combination: including Arginase inhibitors; INCB001158 and the like, but not limited to those.

The compounds include, as single agents or in combination: including Adenosine receptor inhibitors; caffeine, AZD4635, Vipedenant, Preladenant, CPI-444 and the like, but not limited to those.

The compounds include, as single agents or in combination: including cyclooxygenase (COX) 1 and/or 2 inhibitors; Cyclooxygenase (COX) 1 and/or 2 inhibitors; celecoxib, rofecoxib, DuP-697, valdecoxib, etoricoxib, lumiracoxib, indomethacin, 6-methoxy-α-methyl-2-naphthylacetic acid, meclofenamic acid, diclofenac, flufenamic acid, niflumic acid, mefenamic acid, sulindac, tolmetin, suprofen, ketorolac, flurbiprofen, ibuprofen, aceloferac, alcofenac, amfenac, benoxaprofen, bromfenac, carprofen, clidanac, diflunisal, Efenamine, Etodol, fenbufen, fenclofenac, fenclorac, fenoprofen, piroxicam, fleclozic, indoprofen, isofezolac, ketoprofen, loxoprofen, meclofenamate, naproxen, Organoxin, pirprofen, pranoprofen, tolfenamic acid, zaltoprofen, zomepirac and the like, but not limited to those.

The compounds include, as single agents or in combination: including hypoxia inducible factor 1 (HIF-1) inhibitors and hypoxia inducible factor 2 (HIF-2) inhibitors; Chemotomin, Chrysin, Dimethyl-bisphenol, Echinomycin, PX 12, YC-1, Vitexin and the like, but not limited to those.

In yet another embodiment the active pharmaceutical ingredient is a chemokine receptor signal and chemokine receptor modifying drug which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: chemokine receptor signaling modifiers; AZD5069, SX-682, AMD3100, X4P-001, PF-4136309, Maraviroc, LMT-28, madindoline-5 (MDL-5), MDL-16 and MDL-101, SPD-304, C87 ((E)-4-(2-(4-chloro-3-nitrophenyl)), tamatinib fodium (R788), ZINC09609430, ZINC49467549, ZINC13113075, ZINC39907639, ZINC25251930, ZINC02968981, ZINC09544246, ZINC58047088, ZINC72021182, ZINC08704414, ZINC05462670, ZINC35681945, ZINC23553920, ZINC05328058, and ZINC17206695 and the like, but not limited to those.

In yet another embodiment the active pharmaceutical ingredient is a cell cycle checkpoint inhibitor, activator or modulator which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including Checkpoint kinase 1 and/or 2 (CHK1/2) inhibitors; AZD7762, LY 2603618, CCT 241533, NSC 109555, PD 407824, PF 477736, SB 218078, UCN-01, CHIR-124, SAR-020106, CCT244747, SCH900776, V158411, TCS 2312, Hymenialdisine, ABI, NSC1095555, PV1019, VRX0466617, CCT241533, Aminopyridine 7 and the like, but not limited to those.

The compounds include, as single agents or in combination: including Ataxia telangiectasia mutated (ATM) inhibitors; Wortmannin, Caffeine, KU55933, KU55403, KU60019, CP-466722, CGK733, NVP-BEZ235, Torin-2, Methoxyquinazoline 1, Fluoroquinoline 2, SJ573017 and the like, but not limited to those.

The compounds include, as single agents or in combination: including WEE1 inhibitors; PD0166285, PD407824, WEE1 inhibitor II, MK1775, Pyrimidopyrimidinone 8 and the like, but not limited to those.

The compounds include, as single agents or in combination: including Ataxia telangiectasia and Rad3-related (ATR) inhibitors; Schisandrin B, ETP-46464, NU6027, VE-821, VE-822, AZ20, AZD6738 and the like, but not limited to those.

In yet another embodiment the active pharmaceutical ingredient is a cell death inducing chemotherapeutic and immunogenic cell death inducing chemotherapeutics which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: irinotecan hydrochloride, nogitecan hydrochloride, exatecan, RFS-2000, lurtotecan, BNP-1350, Bay-383441, PNU-166148, IDEC-132, BN-80915, DB-38, DB-81, DB-90, DB-91, CKD-620, T-0128, ST-1480, ST-1481, DRF-1042 and DE-310, taxane derivatives such as docetaxel hydrate, IND-5109, BMS-184476, BMS-188797, T-3782, TAX-1011, SB-RA-31012, SBT-1514 and DJ-927, ifosfamide, nimustine hydrochloride, carboquone, cyclophosphamide, dacarbazine, thiotepa, busulfan, melphalan, ranimustine, estramustine phosphate sodium, 6-mercaptopurine riboside, enocitabine, gemcitabine hydrochloride, carmofur, cytarabine, cytarabine ocphosphate, tegafur, doxifluridine, hydroxycarbamide, fluorouracil, methotrexate, mercaptopurine, fludarabine phosphate, actinomycin D, aclarubicin hydrochloride, idarubicin hydrochloride, epirubicin hydrochloride, daunorubicin hydrochloride, pirarubicin hydrochloride, bleomycin hydrochloride, zinostatin stimalamer, neocarzinostatin, mytomycin C, bleomycin sulfate, peplomycin sulfate, vinorelbine tartrate, vincristine sulfate, vindesine sulfate, vinblastine sulfate, amrubicin hydrochloride, gefitinib, exemestan, capecitabine, TNP-470, TAK-165, KW-2401, KW-2170, KW-2871, KT-5555, KT-8391, TZT-1027, S-3304, CS-682, YM-511, YM-598, TAT-59, TAS-101, TAS-102, TA-106, FK-228, FK-317, E7070, E7389, KRN-700, KRN-5500, J-107088, HMN-214, SM-11355, ZD-0473, magnesium 5,10,15,20-tetrakis(4-sulphophenyl)-porphine dodecahydrate, PYROA protein (Emericella nidulans), photosan III, lomefloxacin, cyamemazine, tiaprofenic acid, doxorubicin, mitomycin, paclitaxel, nitrogen mustards, etoposide, camptothecin, 5-fluorouracil, nicotinamide, metronidazole, doxorubicine, Lomeguatrib, Temozolomide, tamoxifen, bleomycin, 5-fluorouracil, cyclophosphamide, methotrexate, gemcitabine, oxaliplatin, cisplatin, carboplatin, camptothecin, CPT-11 (SN-38), Etanidazole, Nimorazole, Mitomycin C, Tirapazamine, procaine, lidocaine, chlorpromazine, Fluordeoxyuridine, bromodeoxyuridine, iododeoxyuridine, hydroxyurea, fludarabine, Texaphyrins (motexafin gadolinium), N-ethylmalemide, paclitaxel, docetaxel, irinotecan, Mechtorethamine, Cyclophosphamide, Ifosfamide, Melphalan, Chlorambucil, Procarbazine (N-methylhydrazine, MIH), Busulfan, Camustine (BCNU), Streptozocin (streptozotocin), Bendamustine, Dacarbazine (DTIC, dimethyttriazenol midazole carboxamide), Temozolomide, Cisplatin, carboplatin, oxaliplatin, Methotrexate (Amethopterin), Pemetrexed, Fluorouracil (5-fluorouracil; 5-FU), capecitabine, Cytarabine (cytosine arabinoside), Gemcitabine, 5-aza-cytidine, Deoxy-5-aza-cytidine, Mercaptoptirine (6-mercaptopurine; 6-MP), Pentostatin (2′-deoxycoformycin), camptothecin, SN-38 (CPT-11), Rudarabine, Clofarabine, Nelarabine, Tirapazamine, Vinblastine, Vinorelbine, Vincristine, Paclitaxel, docetaxel, Etoposide, Teniposide, Topotecan, Irinotecan, Dactinomycin, (actinomycin D). Daunorubicin (daunomycin, rubidomycin), Doxorubicin, Yondelis, Mitoxantrone, Bleomycin, Mitomycin C, L-Asparaginase, Mitotane (o.pDDD) Prednisone, Hydroxyprogesterone caproate, medroxyprogesterone acetate, megestrol acetate, Dietyhlstilbestrol, ethinyl estradiol, Tamoxifen, toremifene, Anastrozole, Gefitinib, letrozole, exemestane, Testosterone propionate, fluoxymesterone, Flutamide, casodex, Leuprolide. Hydroxyurea, Tretinoin, arsenic trioxide, Vorinostat, Imatinib, Dasatinib, nilotinib, Gefrtinib, ertoinib, Sorafenib, Sunitinib, Lapatinib, Bortezomib, Thalidomide, Lenaiidomide, Temsiroiimus, Everolimus, and the like, but not limited to those.

In yet another embodiment the active pharmaceutical ingredient is an osteoinductive or osteogenic drug which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including tiludronate, alendronate, pamidronate, risedronate, ibandronate, zoledronic acid, etidronate, BPH-675, BPH-715, bone morphogenic protein (BMP e.g. BMP-2, BMP-7 and osteogenic protein 1 (OP-1)) and the like, but not limited to those.

In yet another embodiment the active pharmaceutical ingredient is a 3-hydroxy-3-methylglutaryl coenzyme A (HMG-CoA) reductase suppressants (statin) which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including compactin, simvastin and the like, but not limited to those.

In yet another embodiment the active pharmaceutical ingredient is an inducer of osteogenic commitment of stem cells, including BMP, ERK, WNT, AMPK signalling pathways modulators which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including Purmorphamine, Mevinolin, Resveratrol, Icariin, Metformin and the like, but not limited to those.

In yet another embodiment the active pharmaceutical ingredient is an inducer of chondrogenic commitment of stem cells modulators which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including Kartogenin, TD-198.946, Prostaglandin E2 and the like, but not limited to those.

In yet another embodiment the active pharmaceutical ingredient is an inhibitor of matrix metalloproteinases (MMPs) which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including ab142180, ab145190, ab141579, Actinonin, SB-3CT, A4336, Marimastat, TAPI-0, TAPI-1, TAPI-2, Luteolin, Collagenase Inhibitor I, GM 6001, PD166793, Ro 32-3555, CP 471474, UK 356618, NNGH, ND-322, ND-336, RXP470.1 and the like, but not limited to those.

In yet another embodiment the active pharmaceutical ingredient is an interleukin (IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36 and IL-37) modulator which is a ligand for intracellular proteins and/or receptors; or a ligand for cell surface proteins and/or receptors. The drug may serve as a single therapeutic in the gel-forming drug delivery system or serve as part of a combination with one or two additional drugs in the gel-forming drug delivery system. The compounds include, as single agents or in combination: including LMT-28, rapamycin, FK506, A-552, tofacitinib, GSK650394, LTV-1, apilimod, HG-9-91-01, GNE-7915, GSK-J4, I-BET762, SR1001, digoxin, VX-765, ONX0914, SP4206, PD225002, SB265610, PD0210293, PD0220245, NSC201631, NSC61610, LMT-28, rilonacept, anakinra, and NSC80734 and the like, but not limited to those.

In yet another embodiment the composition could further comprise a formulation where one or more of the active pharmaceutical ingredients induces an anti-bacterial effect, or an anti-infectious effect in a human or animal body. The drug may serve as a single therapeutic in the gel-forming drug delivery system or serve as part of a combination with one or two additional drugs in the gel-forming drug delivery system. The compounds include, as single agents or in combination: Aminoglycosides, Amikacin, Gentamicin, Kanamycin, Neomycin, Netilmicin, Tobramycin, Paromomycin, Streptomycin, Spectinomycin(Bs), Ansamycins, Geldanamycin, Herbimycin, Rifaximin, Carbacephem, Loracarbef, Carbapenems, Ertapenem, Doripenem, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefdinir, Cefditoren, Cefotaxime, Cefpodoxime, Ceftibuten, Ceftizoxime, Moxalactam, Ceftriaxone, Cefepime, Ceftaroline fosamil, Ceftobiprole, Glycopeptides, Teicoplanin, Vancomycin, Telavancin, Dalbavancin, Oritavancin, Lincosamides(Bs), Clindamycin, Lincomycin, Lipopeptide, Daptomycin, Macrolides(Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin(Bs), Oxazolidinones(Bs), Linezolid, Posizolid, Radezolid, Torezolid, Penicillins, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Polypeptides, Bacitracin, Colistin, Polymyxin B, Polymyxin E, Ciprofloxacin, Enoxacin, Gatifloxacin, Gemifloxacin, Levofloxacin, Lomefloxacin, Enrofloxacin, Moxifloxacin, Nadifloxacin, Nalidixic acid, Norfloxacin, Ofloxacin, Trovafloxacin, Grepafloxacin, Sparfloxacin, Temafloxacin, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Sulfonamidochrysoidine (archaic), Tetracyclines(Bs), Demeclocycline, Doxycycline, Metacycline, Minocycline, Oxytetracycline, Tetracycline, Clofazimine, Dapsone, Capreomycin, Cycloserine, Ethambutol(Bs), Ethionamide, Isoniazid, Pyrazinamide, Rifampicin, Rifabutin, Rifapentine, Streptomycin, Arsphenamine, Chloramphenicol(Bs), Fosfomycin, Fusidic acid, Metronidazole, Mupirocin, Platensimycin, Quinupristin/Dalfopristin, Thiamphenicol, Tigecycline(Bs), Tinidazole, Trimethoprim(Bs), Vancomycin, Doxycyline, Ceftobiprole[, Ceftaroline, Dalbavancin, Fusidic acid, Mupirocin, Omadacycline, Oritavancin, Tedizolid, Telavancin, Tigecycline, Ceftolozane/tazobactam, etambutol, isoniazid, pyrazinamide, aciclovir, Valaciclovir, efavirenz, emtricitabin, tenofovirdisoproxil, Rilpivirine, penicillin, Trimethoprim-sulfamethoxazole, rifampicin, etambutol, isoniazid, pyrazinamide, voriconazole, amphotericin B, caspofungin, flucytosine, itraconazole, and the like, but not limited to those.

The composition could further comprise a formulation where one or more of the active pharmaceutical ingredients is a chemotherapeutic drug molecule with a molecular weight less than 10 kDa selected from a class of cytotoxic antibiotics, cytotoxic agents, checkpoint inhibitors, immune system modulating agents, anti-cancer agents, anti-inflammatory agents, anti-infectious agents, anesthetic agent, or tissue regeneration agents.

In yet another embodiment, the active pharmaceutical ingredient is an immuno suppressive compound comprising a steroid selected from the group consisting of 21-Acetoxyprefnenolone, Aalclometasone, Algestone, Amicinonide, Beclomethasone, Betamethasone, Betamethasone dipropionate, Betamethasone hemisuccinate, Budesonide, Chloroprednisone, Clobetasol, Blovetasone, Clocortolone, Cloprednol, Corticosterone, Cortisone, Cortivazol, Deflazacort, Desonide, Desoximethasone, dexamethason, Dexamethasone palmitate, Dexamethasone phosphate, Diflorasone, Diflucortolone, Difluprednate, Enoxolone, Fluazacort, Flucloronide, Flumethasone, Flunisolide, Fluocinolone Acetonide, Fludrocortisone, Fluocinonide, Fluocortin Butyl, Fluocortolone, Fluorometholone, Fluperolone, Fluprednidine, Fluprednisolone, Flurandrenolide, Formocortal, Halcinonide, Glucocorticoids, Halomethasone, Halopredone, Hydrocortamate, Hydrocortisone, Limethasone, Mazipredone, Medrysone, Meprednisone, Methyolprednisolone, Methyolprednisolone hemisuccinate, Mometasone Furoate, Paramethasone, Prednicarbate, Prednisolone, Prednisolone palmitate, Prednisolone phosphate, Prednisone, Prednival, Prednylidene, Tixocortal, and Triamcinolone, azathioprine, ciclosporine, 6-mercaptopurine, mycophenolate and the like, but not limited to those.

In yet another embodiment, the active pharmaceutical ingredient compound comprising a small molecule inhibitor acting on intracellular targets selected from the group consisting of c-Fms, PDGFR□, Abl, PDGFR□, NFkB, IkB, JAK1, JAK2, JAK3, GSK3, p38 MAPK, JNK, KIT, EGFR, ERBB2, ERBB4, VEGFR1, VEGFR2, VEGFR3, FLT3, PKC□, RAF1, CDK1, CDK2, CDK4, NLRP3, IRF3, STAT1, STAT2, STAT3, STAT4, STAT5, STATE, Hsp90, Hsp70, PI3K, mTOR, AKT, DNA-PK, ATM, AMPK, PDK-1, S6 kinase, RIP2, TRIF, MYD88, TAK1.

In another embodiment the present invention relates to a composition for use in local co-administration into a human or animal body wherein the composition comprises an active pharmaceutical ingredient, wherein the active pharmaceutical ingredient is an anti-cancer chemotherapeutics selected from the class of compounds that are anti-metabolites, anti-microtubule agents, topoisomerase inhibitors, cytotoxic antibiotics, alkylating agents, radiosensitizers, or are photosensitizers.

In another embodiment the present invention relates to a composition for use in local co-administration into a human or animal body wherein the composition is an x-ray contrast agent for imaging. wherein the ingredient is one or more compounds selected from the group of iodine-based contrast agents, such as lipiodol, CLA-8, CLA-1 and the like, but not limited to those.

The drugs are included in the composition in an amount sufficient to achieve a desired effect. The amount of drug or biologically active agent incorporated into the composition depends upon the desired release profile, the concentration of drug required for a biological effect, and the desired period of release of the drug. The biologically active substance is typically present in the composition in the range from about 0.05 percent to 5 percent or 0.5 percent to about 20 percent by weight relative to the total weight of the composition, and for e.g. antibiotics and the like more typically, between approximately 1 percent to about 15 percent by weight. Another preferred range is from about 2 percent to about 10 percent by weight. For very active agents, such as growth factors, preferred ranges are less than 1% by weight, and less than 0.0001%.

In one embodiment, the present invention relates to treatment of cancerous diseases associated with malignant neoplasia such as malignant neoplasm of lip, mouth or throat, such as malignant neoplasm of the tongue, the base of tongue, gum, floor of mouth, palate, parotid gland, major salivary glands, tonsil, oropharynx, nasopharynx, piriform sinus, hypopharynx or other parts of lip, mouth or throat or malignant neoplasms of digestive organs such as malignant neoplasms of oesophagus, stomach, small intestine, colon, rectosigmoid junction, rectum, anus and anal canal, liver and intrahepatic bile ducts, gallbladder, other parts of biliary tract, pancreas and spleen, malignant neoplasms of respiratory and intrathoracic organs such as malignant neoplasms of the nasal cavity and middle ear, accessory sinuses, larynx, trachea, bronchus and lung, thymus, heart, mediastinum and pleura, malignant neoplasms of bone and articular cartilage such as malignant neoplasm of bone and articular cartilage of limbs, bone and articular cartilage, malignant melanoma of skin, sebaceous glands and sweat glands, malignant neoplasms of mesothelial and soft tissue such as malignant neoplasm of mesothelioma, Kaposi's sarcoma, malignant neoplasm of peripheral nerves and autonomic nervous system, malignant neoplasm of retroperitoneum and peritoneum, malignant neoplasm of connective and soft tissue such as blood vessels, bursa, cartilage, fascia, fat, ligament, lymphatic vessel, muscle, synovia, tendon, head, face and neck, abdomen, pelvis or overlapping lesions of connective and soft tissue, malignant neoplasm of breast or female genital organs such as malignant neoplasms of vulva, vagina, cervix uteri, corpus uteri, uterus, ovary, Fallopian tube, placenta or malignant neoplasms of male genital organs such as malignant neoplasms of penis, prostate, testis, malignant neoplasms of the urinary tract, such as malignant neoplasms of kidney, renal pelvis, ureter, bladder, urethra or other urinary organs, malignant neoplasms of eye, brain and other parts of central nervous system such as malignant neoplasm of eye and adnexa, meninges, brain, spinal cord, cranial nerves and other parts of central nervous system, malignant neoplasms of thyroid and other endocrine glands such as malignant neoplasm of the thyroid gland, adrenal gland, parathyroid gland, pituitary gland, craniopharyngeal duct, pineal gland, carotid body, aortic body and other paraganglia, malignant neoplasms of head, face and neck, thorax, abdomen and pelvis, secondary malignant neoplasm of lymph nodes, respiratory and digestive organs, kidney and renal pelvis, bladder and other and urinary organs, secondary malignant neoplasms of skin, brain, cerebral meninges, or other parts of nervous system, bone and bone marrow, ovary, adrenal gland, malignant neoplasms of lymphoid, haematopoietic and related tissue such as Hodgkin's disease, follicular non-Hodgkin's lymphoma, diffuse non-Hodgkin's lymphoma, peripheral and cutaneous T-cell lymphomas, non-Hodgkin's lymphoma, lymphosarcoma, malignant immunoproliferative diseases such as Waldenström's macroglobulinaemia, alpha heavy chain disease, gamma heavy chain disease, immunoproliferative small intestinal disease, multiple myeloma and malignant plasma cell neoplasms such as plasma cell leukaemia, plasmacytoma, solitary myeloma, lymphoid leukaemia such as acute lymphoblastic leukaemia, myeloid leukaemia, monocytic leukaemia, blast cell leukaemia, stem cell leukaemia, and other and unspecified malignant neoplasms of lymphoid, haematopoietic and related tissue such as Letterer-Siwe disease, malignant histiocytosis, malignant mast cell tumour, true histiocytic lymphoma or other types of malignant neoplasia.

According to the present invention, treatment of carcinoma in situ of oral cavity, oesophagus, stomach, digestive organs, middle ear and respiratory system, melanoma in situ, carcinoma in situ of skin, carcinoma in situ of breast, carcinoma in situ of female or male genitals, carcinoma in situ of bladder, urinary organs or eye, thyroid and other endocrine glands, or other types of carcinoma in situ.

In one embodiment, the present invention relates to treatment of infectious diseases associated with microbial agents, including viruses, rickettsiae, bacteria, fungi, and parasites or other types of infectious agents or any combinations thereof.

In one embodiment, the present invention relates to treatment of diseases associated with radiation therapy, including osteoradionecrosis, soft tissue radionecrosis and radiation induced fibrosis or any combinations thereof.

In one embodiment, the present invention relates to treatment of infectious, degenerative, immune mediated, vascular and inflammatory diseases and non-healing disorders, including, but not limited to, diabetic foot ulcers (DFUs), pressure ulcers, venous leg ulcers and associated complication including bacterial infection, chronic non-healing ulcers, refractory ulcers, gangrene, tissue necrosis, tissue resorption, amputation, proximal limb loss and septicemia or any combinations thereof, osteomyelitis, soft tissue infections, vasculitis, panosteitis, vascular disease, vascular degeneration, limb ischemia, chronic venous insufficiency or any combinations thereof, non-healing bone lesions, non-union of bone fractures, complications after aseptic implant loosening, complications after septic implant loosening, traumatic bone loss, infectious bone loss, resorptive bone loss, osteoporosis, osteonecrosis or any combinations thereof.

### Concentration of Drug Substance in Gel Formulations

### Gels

SuBen and GTO forms homogeneous formulations with EtOH, PC and BnOH. The co-solvent GTO may often be exchanged one to one with GTH, Ethyl-myristate, Ethyl-palmitate or Ethyl-oleate leading to homogeneous gel formulations with alike properties. Likewise, SuBen may be exchanged one to one with alike benzoate carbohydrate esters, such as but not limited to LacBen and Raben. CLA-8 may in addition be substituted for benzoate carbohydrate esters. Homogeneous formulations can therefore be created according to the table below:

Carbohydrate ester=SuBen, LacBen, RaBen, CLA-8 or mixtures thereof. Co-solvent=GTO, GTH, Ethyl-myristate, Ethyl-palmitate, Ethyl-oleate or mixtures thereof. Compositions are given in weight percent or weight ratio.

| | Gel with EtOH | | Gel with PC | | Gel with BnOH | |
|---|---|---|---|---|---|---|
| Chemical | Range | Preferred | Range | Preferred | Range | Preferred |
| Carbohydrate ester | 40-70 | 60 | 40-70 | 52.5 | 40-70 | 55 |
| Co-solvent | 15-30 | 25 | 15-30 | 22.5 | 15-30 | 25 |
| EtOH | 5-30 | 15 | | | | |
| PC | | | 5-30 | 20 | | |
| BnOH | | | | | 5-30 | 20 |
| PLA (10-18 kDa) | 0.25-2 | 0-0.5 | 0.25-2 | 0-0.5 | 0.25-2 | 0-0.5 |

LOIB and GTO form homogeneous formulations with EtOH, PC, BnOH, DMSO and Acetone. The co-solvent GTO may often be exchanged one to one with GTH, Ethyl-myristate, Ethyl-palmitate or Ethyl-oleate leading so homogeneous gel formulations with alike properties. Likewise, LOIB may be exchanged one to one with alike isobutyrate carbohydrate esters, such as but not limited to ROIB, TOIB, SOIB, meLOIB or CLA-1. Homogeneous formulations can therefore be created according to the table below:

Carbohydrate ester=LOIB, ROIB, TOIB, SOIB, meLOIB, CLA-1 or mixtures thereof. Co-solvent=GTO, GTH, Ethyl-myristate, Ethyl-palmitate, Ethyl-oleate or mixtures thereof. Compositions are given in weight percent or weight ratio.

| | Gel with EtOH | | Gel with PC | | Gel with BnOH | | Gel with DMSO | | Gel with Acetone | |
|---|---|---|---|---|---|---|---|---|---|---|
| Chemical | Range | Preferred | Range | Preferred | Range | Preferred | Range | Preferred | Range | Preferred |
| Carbohydrate | 70-85 | 82.5 | 70-85 | 80 | 70-85 | 80 | 70-85 | 80 | 70-85 | 82.5 |
| ester | | | | | | | | | | |
| Co-solvent | 5-15 | 7.5 | 5-15 | 7.5 | 5-15 | 7.5 | 5-15 | 7.5 | 5-15 | 7.5 |
| EtOH | 5-15 | 10 | | | | | | | | |
| PC | | | 5-15 | 12.5 | | | | | | |
| BnOH | | | | | 5-15 | 12.5 | | | | |
| DMSO | | | | | | | 5-15 | 12.5 | | |
| Acetone | | | | | | | | | 5-15 | 10 |

In one embodiment, a gel formulation is a solution said mixture in any combination comprising:
1) Gel forming compounds, such as carbohydrate ester and ether analogues based on mono-, di-, and tri-saccharides such as but not limited to Glucose (dextrose), Fructose (levulose), Galactose, Sibose, Xylose, such as but not limited to Sucrose, Lactulose, Maltose, Trehalose, Cellobiose, Chitobiose, Isomaltise, such as but not limited to Nigerotriose, Maltotrios, Melezitose, Maltotriulose, Raffinose and Kestose. More preferred carbohydrate derivatives are, lactose octapropionate (LOP), lactose octaisobutyrate (LOIB), lactose octabenzoate (LacBen), sucrose octaisobutyrate (SOIB), sucrose octabenzoate (Su Ben), trehalose octaacetate (TOA), trehalose octapropionate (TOP), trehalose octaisobutyrate (TOIB), trehalose octabenzoate (TreBen), raffinose undecaacetate (RUA), raffinose undecapropionate (RUP), raffinose undecaisobutyrate (ROIB), raffinose undecabenzoate (RaBen), such as lactose esters, meLOIB, with methoxy, ethoxy and the like protected anomeric centre, such as iodine rich carbohydrate esters and ethers, CLA-1, CLA-8 and the like.
2) Co-solvents, such as lipid oils such as but not limited to Tripropionin, Tributyrin, glycerol trivalerate, glycerol trihexanoate (GTH), glycerol trioctanoate (GTO), glycerol tridecanoate, glycerol tridodecanoate, or ethyl hexanoate, ethyl octanoate, ethyl decanoate, ethyl laurate, Ethyl myristate, ethyl palmitate, ethyl stearate, ethyl oleate, and naturally derived oils such as but not limited to corn oil, peanut oil, coconut oil, sesame oil, cinnamon oil, soybean oil, lipiodol and poppyseed oil.
3) Solvents, such as polyhydric alcohol such as but not limited to glycerin, diglycerin, polyglycerin, propylene glycol, polypropylene glycol, ethylene glycol, diethylene glycol, triethylene glycol, polyethylene glycol, polyethylene glycol (PEG), benzyl benzoate, triglycerides, acetone, benzyl alcohol (BnOH), ethanol (EtOH), ethyl lactate, propylene carbonate (PC) and Dimethyl Sulfoxide (DMSO), 1-methyl-2-pyrrolidon (NMP), 1-butanol, 2-butanol, Tert-butylmethyl ether, Ethyl ether, Ethyl formate, Heptane, 3-Methyl, 1-butanol, Methylisobutylketone, 2-Methylisobutylketone, 2-Methyl-I-propanol, Pentane, 1-Pentanol, 1-Propanol, 2-Propanol
and is herein referred to as gels

In one embodiment, a gel is a solution said mixture in any weight percent range comprising:

(SOIB:GTO:EtOH) or (SOIB:GTO:DMSO) or (SOIB:GTO:PC) or (SOIB:Lipiodol:EtOH) or (SOIB:Lipiodol:DMSO) or (SOIB:Lipiodol:PC) or (SOIB:Ethyl-palmitate:EtOH) or (SOIB:Ethyl-palmitate:DMSO) or (SOIB:Ethyl-palmitate:PC),

or such as (LOIB:GTO:EtOH) or (LOIB:GTO:DMSO) or (LOIB:GTO:PC) or (LOIB:Lipiodol:EtOH) or (LOIB:Lipiodol:DMSO) or (LOIB: Lipiodol:PC) or (LOIB:Ethyl-palmitate:EtOH) or (LOIB:Ethyl-palmitate:DMSO) or (LOIB:Ethyl-palmitate:PC)

or such as (ROIB:GTO:EtOH) or (ROIB:GTO:DMSO) or (ROIB:GTO:PC) or (ROIB:Lipiodol:EtOH) or (ROIB:Lipiodol:DMSO) or (ROIB:Lipiodol:PC) or (ROIB:Ethyl-palmitate:EtOH) or (ROIB:Ethyl-palmitate:DMSO) or (ROIB:Ethyl-palmitate:PC),

or such as (TOIB:GTO:EtOH) or (TOIB:GTO:DMSO) or (TOIB:GTO:PC) or (TOIB:Lipiodol:EtOH) or (TOIB:Lipiodol:DMSO) or (TOIB:Lipiodol:PC) or (TOIB:Ethyl-palmitate:EtOH) or (TOIB:Ethyl-palmitate: DMSO) or (TOIB:Ethyl-palmitate:PC)

or such as (meLOIB:GTO:EtOH) or (meLOIB:GTO:DMSO) or (meLOIB:GTO:PC) or (meLOIB:Lipiodol:EtOH) or (meLOIB:Lipiodol:DMSO) or (meLOIB:Lipiodol:PC) or (meLOIB:Ethyl-palmitate:EtOH) or (meLOIB:Ethyl-palmitate:DMSO) or (meLOIB:Ethyl-palmitate:PC),

or such as (CLA-1:GTO:EtOH) or (CLA-1:GTO:DMSO) or (CLA-1:GTO:PC) or (CLA-1:GTH:EtOH) or (CLA-1:GTH:DMSO) or (CLA-1:GTH:PC) or (CLA-1:Ethyl-palmitate:EtOH) or (CLA-1:Ethyl-palmitate:DMSO) or (CLA-1:Ethyl-palmitate:PC),

or such as (SuBen:GTO:EtOH) or (SuBen:Lipiodol:EtOH) or (SuBen:Ethyl-palmitate:EtOH) or (SuBen:GTO:PC) or (SuBen:Lipiodol:PC) or (SuBen:Ethyl-palmitate:PC) or (SuBen:GTO:BnOH) or (SuBen:Lipiodol:BnOH) or (SuBen:Ethyl-palmitate:BnOH),

or such as (LacBen:GTO:EtOH) or (LacBen:Lipiodol:EtOH) or (LacBen:Ethyl-palmitate:EtOH) or (LacBen:GTO:PC) or (LacBen:Lipiodol:PC) or (LacBen:Ethyl-palmitate:PC) or (LacBen:GTO:BnOH) or (LacBen:Lipiodol:BnOH) or (LacBen:Ethyl-palmitate:BnOH),

or such as (RaBen:GTO:EtOH) or (RaBen:Lipiodol:EtOH) or (RaBen:Ethyl-palmitate:EtOH) or (RaBen:GTO:PC) or (RaBen:Lipiodol:PC) or (RaBen:Ethyl-palmitate:PC) or (RaBen:GTO:BnOH) or (RaBen:Lipiodol:BnOH) or (RaBen:Ethyl-palmitate:BnOH),

or such as (TreBen:GTO:EtOH) or (TreBen:Lipiodol:EtOH) or (TreBen:Ethyl-palmitate:EtOH) or (TreBen:GTO:PC) or (TreBen:Lipiodol:PC) or (TreBen:Ethyl-palmitate:PC) or (TreBen:GTO:BnOH) or (TreBen:Lipiodol:BnOH) or (TreBen:Ethyl-palmitate:BnOH),

or such as (CLA-8:SuBen:GTO:EtOH) or (CLA-8:SuBen:GTH:EtOH) or (CLA-8:SuBen:Ethyl-palmitate:EtOH) or (CLA-8:SuBen:GTO:PC) or (CLA-8:SuBen:GTH:PC) or (CLA-8:SuBen:Ethyl-palmitate:PC) or (CLA-8:SuBen:GTO:BnOH) or (CLA-8:SuBen:GTH:BnOH) or (CLA-8:SuBen:Ethyl-palmitate:BnOH),

or such as (CLA-8:LacBen:GTO:EtOH) or (CLA-8:LacBen:GTH:EtOH) or (CLA-8:LacBen:Ethyl-palmitate:EtOH) or (CLA-8:LacBen:GTO:PC) or (CLA-8:LacBen:GTH:PC) or (CLA-8:LacBen:Ethyl-palmitate:PC) or (CLA-8:LacBen:GTO:BnOH) or (CLA-8:LacBen:GTH:BnOH) or (CLA-8:LacBen:Ethyl-palmitate: BnOH),

or such as (CLA-8:RaBen:GTO:EtOH) or (CLA-8:RaBen:GTH:EtOH) or (CLA-8:RaBen:Ethyl-palmitate:EtOH) or (CLA-8:RaBen:GTO:PC) or (CLA-8:RaBen:GTH:PC) or (CLA-8:RaBen:Ethyl-palmitate:PC) or (CLA-8:RaBen:GTO:BnOH) or (CLA-8:RaBen:GTH:BnOH) or (CLA-8:RaBen:Ethyl-palmitate:BnOH),

and is herein referred to as gel compositions.

In one embodiment, a gel composition is a solution said mixture with a given weight percent or weight ratio range:

(LOIB:GTO:EtOH) or (LOIB:GTO:DMSO) or (LOIB:GTO:PC) or (LOIB:Lipiodol:EtOH) or (LOIB:Lipiodol:DMSO) or (LOIB:Lipiodol:PC) or (LOIB:Ethyl-palmitate:EtOH) or (LOIB:Ethyl-palmitate:DMSO) or (LOIB:Ethyl-palmitate:PC) wherein 60-90% is LOIB and 0-15% is co-solvent (GTO or Lipiodol or Ethyl-palmitate) and 5-25% is solvent (EtOH or DMSO or PC),

or such as (ROIB:GTO:EtOH) or (ROIB:GTO:DMSO) or (ROIB:GTO:PC) or (ROIB:Lipiodol:EtOH) or (ROIB:Lipiodol:DMSO) or (ROIB:Lipiodol:PC) or (ROIB:Ethyl-palmitate:EtOH) or (ROIB:Ethyl-palmitate:DMSO) or (ROIB:Ethyl-palmitate:PC) wherein 60-90% is ROIB and 0-15% is co-solvent (GTO or Lipiodol or Ethyl-palmitate) and 5-25% is solvent (EtOH or DMSO or PC),

or such as (meLOIB:GTO:EtOH) or (meLOIB:GTO:DMSO) or (meLOIB:GTO:PC) or (meLOIB:Lipiodol:EtOH) or (meLOIB:Lipiodol:DMSO) or (meLOIB:Lipiodol:PC) or (meLOIB:Ethyl-palmitate:EtOH) or (meLOIB:Ethyl-palmitate:DMSO) or (meLOIB:Ethyl-palmitate:PC) wherein 60-90% is meLOIB and 0-15% is co-solvent (GTO or Lipiodol or Ethyl-palmitate) and 5-25% is solvent (EtOH or DMSO or PC),

or such as (CLA-1:GTO:EtOH) or (CLA-1:GTO:DMSO) or (CLA-1:GTO:PC) or (CLA-1:GTH:EtOH) or (CLA-1:GTH:DMSO) or (CLA-1:GTH:PC) or (CLA-1:Ethyl-palmitate:EtOH) or (CLA-1:Ethyl-palmitate:DMSO) or (CLA-1:Ethyl-palmitate:PC) wherein 60-90% is CLA-1, and 0-15% is co-solvent (GTO or GTH or Ethyl-palmitate) and 5-25% is solvent (EtOH or DMSO or PC),

or such as (SuBen:GTO:EtOH) or (SuBen:Lipiodol:EtOH) or (SuBen:Ethyl-palmitate:EtOH) or (SuBen:GTO:PC) or (SuBen:Lipiodol:PC) or (SuBen:Ethyl-palmitate:PC) or (SuBen:GTO:BnOH) or (SuBen:Lipiodol:BnOH) or (SuBen:Ethyl-palmitate:BnOH) wherein 40-80% is SuBen and 0-35% is co-solvent (GTO or Lipiodol or Ethyl-palmitate) and 5-25% is solvent (EtOH or PC or BnOH),

or such as (LacBen:GTO:EtOH) or (LacBen:Lipiodol:EtOH) or (LacBen:Ethyl-palmitate:EtOH) or (LacBen:GTO:PC) or (LacBen:Lipiodol:PC) or (LacBen:Ethyl-palmitate:PC) or (LacBen:GTO:BnOH) or (LacBen:Lipiodol:BnOH) or (LacBen:Ethyl-palmitate:BnOH) wherein 40-80% is LacBen and 0-35% is co-solvent (GTO or Lipiodol or Ethyl-palmitate) and 5-25% is solvent (EtOH or PC or BnOH),

or such as (RaBen:GTO:EtOH) or (RaBen:Lipiodol:EtOH) or (RaBen:Ethyl-palmitate:EtOH) or (RaBen:GTO:PC) or (RaBen:Lipiodol:PC) or (RaBen:Ethyl-palmitate:PC) or (RaBen:GTO:BnOH) or (RaBen:Lipiodol:BnOH) or (RaBen:Ethyl-palmitate:BnOH) wherein 40-80% is RaBen and 0-35% is co-solvent (GTO or Lipiodol or Ethyl-palmitate) and 5-25% is solvent (EtOH or PC or BnOH),

or such as (CLA-8:SuBen:GTO:EtOH) or (CLA-8:SuBen:GTH:EtOH) or (CLA-8:SuBen:Ethyl-palmitate:EtOH) or (CLA-8:SuBen:GTO:PC) or (CLA-8:SuBen:GTH:PC) or (CLA-8:SuBen:Ethyl-palmitate:PC) or (CLA-8:SuBen:GTO:BnOH) or (CLA-8:SuBen:GTH:BnOH) or (CLA-8:SuBen:Ethyl-palmitate:BnOH) wherein 0-20% is CLA-8 and 20-60% is SuBen and 0-35% is co-solvent (GTO or GTH or Ethyl-palmitate) and 5-25% is solvent (EtOH or PC or BnOH),

or such as (CLA-8:LacBen:GTO:EtOH) or (CLA-8:LacBen:GTH:EtOH) or (CLA-8:LacBen:Ethyl-palmitate:EtOH) or (CLA-8:LacBen:GTO:PC) or (CLA-8:LacBen:GTH:PC) or (CLA-8:LacBen:Ethyl-palmitate:PC) or (CLA-8:LacBen:GTO:BnOH) or (CLA-8:LacBen:GTH:BnOH) or (CLA-8:LacBen:Ethyl-palmitate:BnOH) wherein 0-20% is CLA-8 and 20-60% is LacBen, and 0-35% is co-solvent (GTO or GTH or Ethyl-palmitate) and 5-25% is solvent (EtOH or PC or BnOH),

or such as (CLA-8:RaBen:GTO:EtOH) or (CLA-8:RaBen:GTH:EtOH) or (CLA-8:RaBen:Ethyl-palmitate:EtOH) or (CLA-8:RaBen:GTO:PC) or (CLA-8:RaBen:GTH:PC) or (CLA-8:RaBen:Ethyl-palmitate:PC) or (CLA-8:RaBen:GTO:BnOH) or (CLA-8:RaBen:GTH:BnOH) or (CLA-8:RaBen:Ethyl-palmitate:BnOH) wherein 0-20% is CLA-8 and 20-60% is RaBen and 0-35% is co-solvent (GTO or GTH or Ethyl-palmitate) and 5-25% is solvent (EtOH or PC or BnOH),

and is herein referred to as gel compositions.

In one embodiment, a gel composition is a solution said mixture with a given weight percentage or weight ratio range:

(LOIB:GTO:EtOH) or (LOIB:GTO:DMSO) or (LOIB:GTO:PC) wherein 82.5% is LOIB and 7.5% is GTO co-solvent and 10% is solvent (EtOH or DMSO or PC),

or such as (meLOIB:GTO:EtOH) or (meLOIB:GTO:DMSO) or (meLOIB:GTO:PC) wherein 82.5% is meLOIB and 7.5% is GTO co-solvent and 10% is solvent (EtOH or DMSO or PC),

or such as (CLA-1:GTO:EtOH) or (CLA-1:GTO:DMSO) or (CLA-1:GTO:PC) wherein 82.5% is CLA-1, and 7.5% is GTO co-solvent and 10% is solvent (EtOH or DMSO or PC),

or such as (SuBen:GTO:EtOH) or (SuBen:Lipiodol:EtOH) or (SuBen:Ethyl-palmitate:EtOH) wherein 60% is SuBen and 25% is co-solvent (GTO or Lipiodol or Ethyl-palmitate) and 15% is EtOH solvent,

or such as (LacBen:GTO:EtOH) or (LacBen:Lipiodol:EtOH) or (LacBen:Ethyl-palmitate:EtOH) wherein 60% is LacBen and 25% is co-solvent (GTO or Lipiodol or Ethyl-palmitate) and 15% is EtOH solvent,

or such as (RaBen:GTO:EtOH) or (RaBen:Lipiodol:EtOH) or (RaBen:Ethyl-palmitate:EtOH) wherein 60% is RaBen and 25% is co-solvent (GTO or Lipiodol or Ethyl-palmitate) and 15% is solvent EtOH,

or such as (CLA-8:SuBen:GTO:EtOH) or (CLA-8:SuBen:GTH:EtOH) or (CLA-8:SuBen:Ethyl-palmitate:EtOH) wherein 20% is CLA-8 and 40% is SuBen and 25% is co-solvent (GTO or GTH or Ethyl-palmitate) and 15% is EtOH solvent,

or such as (CLA-8:LacBen:GTO:EtOH) or (CLA-8:LacBen:GTH:EtOH) or (CLA-8:LacBen:Ethyl-palmitate:EtOH) wherein 20% is CLA-8 and 40% is LacBen, 25% is co-solvent (GTO or GTH or Ethyl-palmitate) and 15% is EtOH solvent,

or such as (CLA-8:RaBen:GTO:EtOH) or (CLA-8:RaBen:GTH:EtOH) or (CLA-8:RaBen:Ethyl-palmitate:EtOH) wherein 20% is CLA-8 and 40% is RaBen and 25% is co-solvent (GTO or GTH or Ethyl-palmitate) and 5-25% is EtOH solvent,

and is herein referred to as gel compositions.

In one embodiment, a gel has the composition (LOIB:GTO:EtOH) and is herein referred to as LOIB gel.

In one embodiment, a gel has the composition (meLOIB:GTO:EtOH) and is herein referred to as meLOIB gel.

In one embodiment, a gel has the composition (SuBen:GTO:EtOH:PLA) and is herein referred to as SuBen gel.

In one embodiment, a gel has the composition (SuBen:GTO:EtOH) and is herein referred to as nSuBen gel.

In one embodiment, a gel has the composition (RaBen:GTO:EtOH) and is herein referred to as RaBen gel.

In one embodiment, a gel has the composition (LacBen:GTO:EtOH) and is herein referred to as LacBen gel.

In one embodiment, a gel has the composition (CLA-8:SuBen:GTO:EtOH) and is herein referred to as CLA-SuBen gel.

In one embodiment, a gel has the composition (CLA-8:RaBen:GTO:EtOH) and is herein referred to as CLA-RaBen gel.

In one embodiment, a gel has the composition (CLA-8:LacBen:GTO:EtOH) and is herein referred to as CLA-LacBen gel.

In one embodiment, a gel has the composition (meLOIB:GTO:EtOH) wherein meLOIB is 82.5% and GTO is 7.5% and EtOH is 10%, and is herein referred to as meLOIB 82.5% gel.

In one embodiment, a gel has the composition (SuBen:GTO:EtOH) wherein SuBen is 60% and GTO is 25% and EtOH is 15% and is herein referred to as nSuBen 60% gel.

In one embodiment, a gel has the composition (RaBen:GTO:EtOH) wherein RaBen is 60% and GTO is 25% and EtOH is 15% and is herein referred to as RaBen 60% gel.

In one embodiment, a gel has the composition (LacBen:GTO:EtOH) wherein LacBen is 60% and GTO is 25% and EtOH is 15%, and is herein referred to as LacBen 60% gel.

In one embodiment, a gel has the composition (CLA-8:SuBen:GTO:EtOH) wherein CLA-8 is 20% and SuBen is 40% and GTO is 25% and EtOH is 15%, and is herein referred to as CLA-SuBen 40% gel.

In one embodiment, a gel has the composition (CLA8:RaBen:GTO:EtOH) wherein CLA-8 is 20% and RaBen is 40% and GTO is 25% and EtOH is 15%, and is herein referred to as CLA-RaBen 40% gel.

In one embodiment, a gel has the composition (CLA-8:LacBen:GTO:EtOH) wherein CLA-8 is 20% and SuBen is 40% and GTO is 25% and EtOH is 15%, and is herein referred to as CLA-LacBen 40% gel.

In one embodiment the gel has the composition LOIB:GTO:EtOH (82.5:7.5:10 weight %) and is herein referred to as LOIB 82.5%.

In one embodiment the gel has the composition SuBen:PLA:GTO:EtOH (59.5:0.5:25:15 weight %) and is herein referred to as SuBen 60%.

Experimentally obtained drug solubilities are given in the table (all percentages are weight %).

| | Solubility | |
|---|---|---|
| Drugs | (mg/g gel) | Gel composition |
| Resiquimod | >10 | e.g LOIB 82.5%, SuBen 60% |
| Imiquimod | >4.8 | e.g SuBen 60% |
| Gardiquimod | >1.5 | e.g LOIB 82.5%, SuBen 60% |
| RepSox | >8 | e.g LOIB 82.5%, SuBen 60% |
| Galunisertib | >8 | e.g LOIB 82.5%, SuBen 60% |
| SD-208 | >8 | e.g SuBen 60% |
| SD-208 | >8 | e.g SuBen:LOIB 35%/35% |
| SHP099 | >1 | e.g SuBen 60% |

In one embodiment the gel forming composition contains at least one active pharmaceutical ingredient selected from a class of chemotherapeutics, ICDs, TGFβ inhibitors, IFN agonists, IDO inhibitors, GSK inhibitors, RIG-I agonists, SHP inhibitors, SHP2 inhibitors, ICD inducers, Sting agonists, PD1 or PD-L1 inhibitors, CTLA4 inhibitors, OX40 agonist, CD40 agonist, CD137 agonist, GITR agonist, TNFSFR agonist or WNT/b-catenin inhibitors is dissolved.

In one embodiment, the gel forming composition comprises a LOIB gel or Suben gel or meLOIB gel or nSuBen gel or RaBen gel or LacBen gel or CLA-SuBen gel or CLA-RaBen gel or CLA-LacBen gel wherein at least one active pharmaceutical ingredient is dissolved selected from a class of chemotherapeutics, ICDs, TGFβ inhibitors, IFN agonists, IDO inhibitors, GSK inhibitors, GSK inhibitors, RIG-I agonists, SHP inhibitors, SHP2 inhibitors, ICD inducers, Sting agonists, PD1 or PD-L1 inhibitors, CTLA4 inhibitors, OX40 agonist, CD40 agonist, CD137 agonist, GITR agonist, TNFSFR agonist or WNT/b-catenin inhibitors is dissolved

In one embodiment the gel forming composition contains at least one active pharmaceutical ingredient selected from a class of antimicrobial agents including antibibiotics, inflammation modulating drugs, interleukin inhibitors and interleukin agonists, immunosuppressive agents acute phase protein inhibiting drugs, metalloproteinases (MMP) Osteoconductive and osteoinductive agents, Glycogen synthase kinase 3b inhibitors, transforming growth factor beta-1-3 agonists, hydroxy-3-methylglutaryl coenzyme A (HMG-CoA) reductase suppressants (statin, osteogenic commitment of stem cells, including BMP, ERK, WNT, AMPK signalling pathways modulators, inducer of chondrogenic commitment of stem cells modulators, inducer of osteogenic commitment of stem cells modulators is dissolved.

In one embodiment, the gel forming composition comprises a LOIB gel or Suben gel or meLOIB gel or nSuBen gel or RaBen gel or LacBen gel or CLA-SuBen gel or CLA-RaBen gel or CLA-LacBen gel wherein at least one active pharmaceutical ingredient is dissolved selected from a class of antimicrobial agents including antibibiotics, inflammation modulating drugs, inhibitors and activators of interleukin, inhibitors and agonists, immunosuppressive agents acute phase protein inhibiting drugs, metalloproteinases (MMP) Osteoconductive and osteoinductive agents, Glycogen synthase kinase 3b inhibitors, transforming growth factor beta-1-3 agonists, hydroxy-3-methylglutaryl coenzyme A (HMG-CoA) reductase suppressants (statin, osteogenic commitment of stem cells, including BMP, ERK, WNT, AMPK signalling pathways modulators, inducer of chondrogenic commitment of stem cells modulators, inducer of osteogenic commitment of stem cells modulators is dissolved.

### Monogels (TLRs)

In one embodiment, LOIB gel or Suben gel or meLOIB gel or nSuBen gel or RaBen gel or LacBen gel or CLA-SuBen gel or CLA-RaBen gel or CLA-LacBen gel contains an amount of a TLR agonist.

In one embodiment, LOIB 82.5% gel or Suben 60% gel contains 0.1-10 mg/g R848, such as 0.5-5 mg/g R848, for example 1-4 mg/g R848, such as 1.2-2.4 mg/g R848.

In one embodiment, LOIB 82.5% gel or Suben 60% gel contains 0.1-10 mg/g Imiquimod, such as 2-8 mg/g Imiquimod, for example 2-6 mg/g Imiquimod, such as 2.4-4.8 mg/g Imiquimod.

In one embodiment, LOIB 82.5% gel or Suben 60% gel contains 0.1-10 mg/g Gardiquimod, such as 2-8 mg/g gardiquimod, for example 2-6 mg/g Gardiquimod, such as 2.4-4.8 mg/g Gardiquimod.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TLR agonist such as a TLR3, TLR4, TLR7, TLR8 or TLR9 agonist.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of at least two TLR agonist such as a TLR3, TLR4, TLR7, TLR8 or TLR9 agonists.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-10 mg/g R848, Imiquimod (R837), Resiquimod (R848), and the like, but not limited to those.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a RIG-1 agonist such as KIN1148, KIN131A, KIN126X, KIN150X, KIN1000, KIN1408, SLR14, MK4621, RGT100, KIN1400 and the like, but not limited to those.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a STING agonist such as ADU-S100, MK-1454, PO-424, H-151, C-176 and the like, but not limited to those.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a cell death inducing chemotherapeutic.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell death inducing chemotherapeutic such as, daunorubicin, vinblastine sulfate, amrubicin hydrochloride, gefitinib, exemestan, capecitabine, 5-fluorouracil, doxorubicine, 5-aza-cytidine, Deoxy-5-aza-cytidine, Paclitaxel, docetaxel, Etoposide, Daunorubicin, Yondelis, Mitoxantrone, Bortezomib, and the like, but not limited to those.

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-200 mg/g of a anti-bacterial effect, or an anti-infectious effect agent such as Carbacephem, Azithromycin, Clarithromycin, Erythromycin, Colistin, Polymyxin B, Polymyxin E, Ciprofloxacin, Enoxacin, Gatifloxacin, Gemifloxacin, Levofloxacin, Lomefloxacin, Enrofloxacin, Rifampin, Rifampicin, Vancomycin, Fusidic acid, acyclovir, itraconazole, and the like, but not limited to those.

### Combigels (RIG-1 and TLR)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a RIG-1 agonist and a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a RIG-1 agonist such as KIN1408, SLR14, MK4621, RGT100 or KIN1400 and 0.1-10 mg/g of a TLR agonist such as R848, Imiquimod (R837) or Resiquimod (R848), and the like, but not limited to those.

### Combigels (TLR and TGFß Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and a TLR agonist.

In one embodiment, LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and a TLR/7 agonist.

In one embodiment, LOIB 82.5% gel or Suben 60% gel contains an amount of TGFβ signaling inhibitors and a TLR/7 agonist.

In one embodiment, LOIB 82.5% gel or Suben 60% contains 1.2-2.4 mg/g R848 and 1-20 mg/g RepSox, such as 3-15 mg/g RepSox, for example 4-12 mg/g RepSox, such as 6-10 mg/g RepSox.

In one embodiment, LOIB 82.5% gel or Suben 60% contains 1-4 mg/g R848 and 1-20 mg/g RepSox, such as 3-15 mg/g RepSox, for example 4-12 mg/g RepSox, such as 6-10 mg/g RepSox.

In one embodiment, LOIB 82.5% gel or Suben 60% contains 2-8 mg/g Imiquimod and 1-20 mg/g RepSox, such as 3-15 mg/g RepSox, for example 4-12 mg/g RepSox, such as 6-10 mg/g RepSox.

In one embodiment, LOIB 82.5% gel or Suben 60% contains 1.2-2.4 mg/g Gardiquimod and 1-20 mg/g RepSox, such as 3-15 mg/g RepSox, for example 4-12 mg/g RepSox, such as 6-10 mg/g RepSox.

In one embodiment, LOIB 82.5% gel or Suben 60% contains 1.2-2.4 mg/g R848 and 1-20 mg/g Galunisertib, such as 3-15 mg/g Galunisertib, for example 4-12 mg/g Galunisertib, such as 6-10 mg/g Galunisertib.

In one embodiment, LOIB 82.5% gel or Suben 60% contains 1˜4 mg/g R848 and 1-20 mg/g Galunisertib, such as 3-15 mg/g Galunisertib, for example 4-12 mg/g Galunisertib, such as 6-10 mg/g Galunisertib.

In one embodiment, LOIB 82.5% gel or Suben 60% contains 2-8 mg/g Imiquimod and 1-20 mg/g Galunisertib, such as 3-15 mg/g Galunisertib, for example 4-12 mg/g Galunisertib, such as 6-10 mg/g Galunisertib.

In one embodiment, LOIB 82.5% gel or Suben 60% contains 1.2-2.4 mg/g Gardiquimod and 1-20 mg/g SD-208, such as 3-15 mg/g SD-208, for example 4-12 mg/g SD-208, such as 6-10 mg/g SD-208.

In one embodiment, LOIB 82.5% gel or Suben 60% contains 1.2-2.4 mg/g R848 and 1-20 mg/g SD-208, such as 3-15 mg/g SD-208, for example 4-12 mg/g SD-208, such as 6-10 mg/g SD-208.

In one embodiment, LOIB 82.5% gel or Suben 60% contains 1-4 mg/g R848 and 1-20 mg/g SD-208, such as 3-15 mg/g SD-208, for example 4-12 mg/g SD-208, such as 6-10 mg/g SD-208.

In one embodiment, LOIB 82.5% gel or Suben 60% contains 2-8 mg/g Imiquimod and 1-20 mg/g SD-208, such as 3-15 mg/g SD-208, for example 4-12 mg/g SD-208, such as 6-10 mg/g SD-208.

In one embodiment, LOIB 82.5% gel or Suben 60% contains 1.2-2.4 mg/g Gardiquimod and 1-20 mg/g SD-208, such as 3-15 mg/g SD-208, for example 4-12 mg/g SD-208, such as 6-10 mg/g SD-208.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TGFβ signaling inhibitor such as RepSox, Galunisertib (LY2157299), LY550410 or SD-208 and 0.1-10 mg/g of a TLR agonist such as R848, Imiquimod (R837) or Resiquimod (R848), and the like, but not limited to those.

### Combigels (TLR and GSK Inhibitors)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of GSK signaling inhibitors and a TLR agonist.

In one embodiment, LOIB 82.5% gel or Suben 60% gel contains an amount of GSK signaling inhibitors and a TLR/7 agonist.

In one embodiment, LOIB 82.5% gel or Suben 60% contains 1.2-2.4 mg/g R848 and 1-20 mg/g CHIR99021, PD0325901, TWS119, SB415286 or GSK-3 inhibitor-X, such as 3-15 mg/g CHIR99021, PD0325901, TWS119, SB415286 or GSK-3 inhibitor-X, for example 4-12 mg/g CHIR99021, PD0325901, TWS119, SB415286 or GSK-3 inhibitor-X, such as 6-10 mg/g CHIR99021, PD0325901, TWS119, SB415286 or GSK-3 inhibitor-X.

In one embodiment, LOIB 82.5% gel or Suben 60% contains 1-4 mg/g R848 and 1-20 mg/g CHIR99021, PD0325901, TWS119, SB415286 or GSK-3 inhibitor-X, such as 3-15 mg/g CHIR99021, PD0325901, TWS119, SB415286 or GSK-3 inhibitor-X, for example 4-12 mg/g CHIR99021, PD0325901, TWS119, SB415286 or GSK-3 inhibitor-X, such as 6-10 mg/g CHIR99021, PD0325901, TWS119, SB415286 or GSK-3 inhibitor-X.

In one embodiment, LOIB 82.5% gel or Suben 60% contains 2-8 mg/g Imiquimod and 1-20 mg/g CHIR99021, PD0325901, TWS119, SB415286 or GSK-3 inhibitor-X, such as 3-15 mg/g CHIR99021, PD0325901, TWS119, SB415286 or GSK-3 inhibitor-X, for example 4-12 mg/g CHIR99021, PD0325901, TWS119, SB415286 or GSK-3 inhibitor-X, such as 6-10 mg/g CHIR99021, PD0325901, TWS119, SB415286 or GSK-3 inhibitor-X.

In one embodiment, LOIB 82.5% gel or Suben 60% contains 1.2-2.4 mg/g Gardiquimod and 1-20 mg/g CHIR99021, PD0325901, TWS119, SB415286 or GSK-3 inhibitor-X, such as 3-15 mg/g CHIR99021, PD0325901, TWS119, SB415286 or GSK-3 inhibitor-X, for example 4-12 mg/g CHIR99021, PD0325901, TWS119, SB415286 or GSK-3 inhibitor-X, such as 6-10 mg/g CHIR99021, PD0325901, TWS119, SB415286 or GSK-3 inhibitor-X.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-10 mg/g of a TLR agonist such as R848, Imiquimod (R837) or Resiquimod (R848) and 1-20 mg/g CHIR99021, PD0325901, TWS119, SB415286 or GSK-3 inhibitor-X and the like, but not limited to those.

### Combigels (TLR and IDO Inhibitors)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of IDO signaling inhibitors and a TLR agonist.

In one embodiment, LOIB 82.5% gel or Suben 60% gel contains an amount of IDO signaling inhibitors and a TLR/7 agonist.

In one embodiment, LOIB 82.5% gel or Suben 60% contains 1.2-2.4 mg/g R848 and 1-20 mg/g NLG919, such as 3-15 mg/g NLG919, for example 4-12 mg/g NLG919, such as 6-10 mg/g NLG919.

In one embodiment, LOIB 82.5% gel or Suben 60% contains 1-4 mg/g R848 and 1-20 mg/g NLG919, such as 3-15 mg/g NLG919, for example 4-12 mg/g NLG919, such as 6-10 mg/g NLG919.

In one embodiment, LOIB 82.5% gel or Suben 60% contains 2-8 mg/g Imiquimod and 1-20 mg/g NLG919, such as 3-15 mg/g NLG919, for example 4-12 mg/g NLG919, such as 6-10 mg/g NLG919.

In one embodiment, LOIB 82.5% gel or Suben 60% contains 1.2-2.4 mg/g Gardiquimod and 1-20 mg/g NLG919, such as 3-15 mg/g NLG919, for example 4-12 mg/g NLG919, such as 6-10 mg/g NLG919.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 1-10 mg/g R848, Imiquimod or Gardiquimod and 1-20 mg/g NLG919, and the like, but not limited to those.

### Combigels (TLRs and Interferon Agonists)

In one embodiment, LOIB 82.5% gel or Suben 60% gel contains an amount of Interferon agonist and a TLR/7 agonist.

In one embodiment, LOIB 82.5% gel or Suben 60% contains 1.2-2.4 mg/g R848 and 1-20 mg/g R08181, such as 3-15 mg/g R08181, for example 4-12 mg/g R08181, such as 6-10 mg/g R08181.

In one embodiment, LOIB 82.5% gel or Suben 60% contains 1˜4 mg/g R848 and 1-20 mg/g R08181, such as 3-15 mg/g R08181, for example 4-12 mg/g R08181, such as 6-10 mg/g R08181.

In one embodiment, LOIB 82.5% gel or Suben 60% contains 2-8 mg/g Imiquimod and 1-20 mg/g R08181, such as 3-15 mg/g R08181, for example 4-12 mg/g R08181, such as 6-10 mg/g R08181.

In one embodiment, LOIB 82.5% gel or Suben 60% contains 1.2-2.4 mg/g Gardiquimod and 1-20 mg/g R08181, such as 3-15 mg/g R08181, for example 4-12 mg/g R08181, such as 6-10 mg/g R08181.

### Combigels (TLRs and SHP2 or ICDs)

In one embodiment, LOIB 82.5% gel or Suben 60% gel contains an amount of SHP2 inhibitor or ICD drug and a TLR/7 agonist.

In one embodiment, LOIB 82.5% gel or Suben 60% contains 1.2-2.4 mg/g R848 and 1-20 mg/g SHP099, PC-61275, Mitoxantrone, Bortezomib, Crizotinib, Oxaliplatin, Doxorubicin or Cyclophosphamide, such as 0.5-8 mg/g SHP099, PC-61275, Mitoxantrone, Bortezomib, Crizotinib, Oxaliplatin, Doxorubicin or Cyclophosphamide, for example 0.8-6 mg/g SHP099, PC-61275, Mitoxantrone, Bortezomib, Crizotinib, Oxaliplatin, Doxorubicin or Cyclophosphamide such as 1-4 mg/g SHP099, PC-61275, Mitoxantrone, Bortezomib, Crizotinib, Oxaliplatin, Doxorubicin or Cyclophosphamide.

In one embodiment, LOIB 82.5% gel or Suben 60% contains 1˜4 mg/g R848 and 1-20 mg/g SHP099, PC-61275, Mitoxantrone, Bortezomib, Crizotinib, Oxaliplatin, Doxorubicin or Cyclophosphamide, such as 0.5-8 mg/g SHP099, PC-61275, Mitoxantrone, Bortezomib, Crizotinib, Oxaliplatin, Doxorubicin or Cyclophosphamide, for example 0.8-6 mg/g SHP099, PC-61275, Mitoxantrone, Bortezomib, Crizotinib, Oxaliplatin, Doxorubicin or Cyclophosphamide such as 1-4 mg/g SHP099, PC-61275, Mitoxantrone, Bortezomib, Crizotinib, Oxaliplatin, Doxorubicin or Cyclophosphamide.

In one embodiment, LOIB 82.5% gel or Suben 60% contains 2-8 mg/g Imiquimod and 1-20 mg/g SHP099, PC-61275, Mitoxantrone, Bortezomib, Crizotinib, Oxaliplatin, Doxorubicin or Cyclophosphamide, such as 0.5-8 mg/g SHP099, PC-61275, Mitoxantrone, Bortezomib, Crizotinib, Oxaliplatin, Doxorubicin or Cyclophosphamide, for example 0.8-6 mg/g SHP099, PC-61275, Mitoxantrone, Bortezomib, Crizotinib, Oxaliplatin, Doxorubicin or Cyclophosphamide such as 1-4 mg/g SHP099, PC-61275, Mitoxantrone, Bortezomib, Crizotinib, Oxaliplatin, Doxorubicin or Cyclophosphamide.

In one embodiment, LOIB 82.5% gel or Suben 60% contains 1.2-2.4 mg/g Gardiquimod and 1-20 mg/g SHP099, PC-61275, Mitoxantrone, Bortezomib, Crizotinib, Oxaliplatin, Doxorubicin or Cyclophosphamide, such as 0.5-8 mg/g SHP099, PC-61275, Mitoxantrone, Bortezomib, Crizotinib, Oxaliplatin, Doxorubicin or Cyclophosphamide, for example 0.8-6 mg/g SHP099, PC-61275, Mitoxantrone, Bortezomib, Crizotinib, Oxaliplatin, Doxorubicin or Cyclophosphamide such as 1-4 mg/g SHP099, PC-61275, Mitoxantrone, Bortezomib, Crizotinib, Oxaliplatin, Doxorubicin or Cyclophosphamide.

### Combigels (TLR and SHP2)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of SHP2 inhibitor and an amount of a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 1-10 mg/g R848, Imiquimod or Gardiquimod and 1-20 mg/g SHP099 or PC-61275, and the like, but not limited to those.

### Combigels (TLRs and Cell Death Inducing Chemotherapeutic)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount of a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell death inducing chemotherapeutic such as, daunorubicin, vinblastine sulfate, amrubicin hydrochloride, gefitinib, exemestan, capecitabine, 5-fluorouracil, doxorubicine, 5-aza-cytidine, Deoxy-5-aza-cytidine, Paclitaxel, docetaxel, Etoposide, Daunorubicin, Yondelis, Mitoxantrone or Bortezomib, and the like, but not limited to those and 0.1-10 mg/g of a TLR agonist such as R848, Imiquimod (R837) or Resiquimod (R848), and the like, but not limited to those.

### Combigels (TLR and Immune Checkpoint Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor and an amount of a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor such as BMS-8, INCMGA00012, CX072, CCX4503 and the like, but not limited to those and 0.1-0.1-10 mg/g of a TLR agonist such as R848, Imiquimod (R837) or Resiquimod (R848), and the like, but not limited to those.

### Combigels (TLR and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TNFSFR agonist, such as a OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains an 0.1-20 mg/g of a TNFSFR agonist, such as a OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those and 0.1-10 mg/g of a TLR agonist such as R848, Imiquimod (R837) or Resiquimod (R848), and the like, but not limited to those.

### Combigels (TLR and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a Wnt/6-catenin inhibitor and an amount of a TLR agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Wnt/6-catenin inhibitor such as WAY-316606 or XAV939 and the like, but not limited to those and 1-10 mg/g of a TLR agonist such as R848, Imiquimod or Gardiquimod and the like, but not limited to those.

### Combigels (RIG-1 and TGFβ□ Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TGFβ signaling inhibitor such as RepSox, Galunisertib (LY2157299) or LY550410, and the like, but not limited to those and 0.1-20 mg/g of a RIG-1 agonist such as KIN1408, SLR14, MK4621, RGT100 or KIN1400 and the like, but not limited to those.

### Combigels (RIG-1 and GSK Inhibitors)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of GSK signaling inhibitors and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains an amount 0.1-20 mg/g of GSK signaling inhibitors such as TWS119 or SB415286-and 0.1-20 mg/g of a RIG-1 agonist such as KIN1408, SLR14, MK4621, RGT100 or KIN1400 and the like, but not limited to those.

### Combigels (RIG-1 and IDO Inhibitors)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of IDO inhibitor and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of an IDO inhibitor such as methyl-tryptophan, D-1MT or BMS-986205 and the like, but not limited to those and 0.1-20 mg/g of a RIG-1 agonist such as KIN1408, SLR14, MK4621, RGT100 or KIN1400 and the like, but not limited to those.

### Combigels (RIG-1 and SHP2)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of SHP2 inhibitor and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/ of SHP2 inhibitor such as SHP099 or PC-61275 and 0.1-20 mg/g of a RIG-1 agonist such as KIN1408, SLR14, MK4621, RGT100 or KIN1400 and the like, but not limited to those.

### Combigels (RIG-1 and Cell Death Inducing Chemotherapeutic)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of cell death inducing chemotherapeutic such as, daunorubicin, vinblastine sulfate, amrubicin hydrochloride, gefitinib, exemestan, capecitabine, 5-fluorouracil, doxorubicine, 5-aza-cytidine, Deoxy-5-aza-cytidine, Paclitaxel, docetaxel, Etoposide, Daunorubicin, Yondelis, Mitoxantrone or Bortezomib, and the like, but not limited to those and 0.1-20 mg/g of a RIG-1 agonist such as KIN1408, SLR14, MK4621, RGT100 or KIN1400 and the like, but not limited to those.

### Combigels (RIG-1 and Immune Checkpoint Inhibitor)

In one embodiment, the gel forming composition LOIB gel or Suben gel or meLOIB gel or nSuBen gel or RaBen gel or LacBen gel or CLA-SuBen gel or CLA-RaBen gel or CLA-LacBen gel contains an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor such as BMS-8, INCMGA00012, CX072 or CCX4503 and the like, but not limited to those and 0.1-20 mg/g of a RIG-1 agonist such as KIN1408, SLR14, MK4621, RGT100 or KIN1400 and the like, but not limited to those.

### Combigels (RIG-1 and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of a RIG-1 agonist.

In one embodiment, LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those and 0.1-20 mg/g of a RIG-1 agonist such KIN1408, SLR14, MK4621, RGT100 or KIN1400 and the like, but not limited to those.

### Combigels (RIG-1 and Wnt/6-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a Wnt/6-catenin inhibitor and an amount of a RIG-1 agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Wnt/6-catenin inhibitor such as WAY-316606 or XAV939, and the like, but not limited to those and 0.1-20 mg/g of a RIG-1 agonist such as KIN1408, SLR14, MK4621, RGT100 or KIN1400 and the like, but not limited to those.

### Combigels (STING and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those and 0.1-20 mg/g of a STING agonist such as ADU-S100, MK-1454, PO-424, H-151 or C-176 and the like, but not limited to those.

### Combigels (STING and Immune Checkpoint Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor ro Lymphocyte activating gene 3 (LAG3) inhibitor and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor such as BMS-8, INCMGA00012, CX072 or CCX4503 and the like, but not limited to those and 0.1-20 mg/g of a STING agonist such as ADU-S100, MK-1454, PO-424, H-151 or C-176 and the like, but not limited to those.

### Combigels (STING and TGFß Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFß inhibitor inhibitor and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TGFβ signaling inhibitor such as RepSox, Galunisertib (LY2157299) or LY550410, and the like, but not limited to those and 0.1-20 mg/g of a STING agonist such as ADU-S100, MK-1454, PO-424, H-151 or C-176 and the like, but not limited to those.

### Combigels (STING and SHP2 Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a SHP2 inhibitor inhibitor and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/ of SHP2 inhibitor such as SHP099 or PC-61275 and the like, but not limited to those and 0.1-20 mg/g of a STING agonist such as ADU-S100, MK-1454, PO-424, H-151 or C-176 and the like, but not limited to those.

### Combigels (STING and IDO Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an IDO inhibitor and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of an IDO inhibitor such as methyl-tryptophan, D-1MT or BMS-986205 and the like, but not limited to those and 0.1-20 mg/g of a STING agonist such as ADU-S100, MK-1454, PO-424, H-151 or C-176 and the like, but not limited to those.

### Combigels (STING and Wnt/6-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a Wnt/6-catenin inhibitor and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Wnt/6-catenin inhibitor such as WAY-316606 or XAV939, and the like, but not limited to those and 0.1-20 mg/g of a STING agonist such as ADU-S100, MK-1454, PO-424, H-151 or C-176 and the like, but not limited to those.

### Combigels (STING and Cell Death Inducing Chemotherapeutic)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a cell death inducing chemotherapeutic and an amount of a STING agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of cell death inducing chemotherapeutic such as such as, daunorubicin, vinblastine sulfate, amrubicin hydrochloride, gefitinib, exemestan, capecitabine, 5-fluorouracil, doxorubicine, 5-aza-cytidine, Deoxy-5-aza-cytidine, Paclitaxel, docetaxel, Etoposide, Daunorubicin, Yondelis, Mitoxantrone or Bortezomib, and the like, but not limited to those and 0.1-20 mg/g of a STING agonist such as ADU-S100, MK-1454, PO-424, H-151 or C-176 and the like, but not limited to those.

### Combigels (Antibiotic and MMP Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of an inhibitor of matrix metalloproteinases (MMP).

### Combigels (Antibiotic and Osteoinductive or Osteogenic Drug)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of an osteoinductive or osteogenic drug.

### Combigels (Antibiotic and Hydroxy-3-Methylglutaryl Coenzyme a (HMG-CoA) Reductase Suppressants)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of a hydroxy-3-methylglutaryl coenzyme A (HMG-CoA) reductase suppressants drug.

### Combigels (TLR and Cell Death Inducing Chemotherapeutic and TGFß Inhibitor

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and a TLR agonist and an amount of a TGFß inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell deatch inducing chemotherapeutic such as Doxorubcin or mitoxantone and the like, but not limited to those and a TLR agonist such as 0.1-10 mg/g R848 and the like, but not limited to those and a TGFß inhibitor such as 0.1-20 mg/g RepSox or SD-208 and the like, but not limited to those.

### Combigels (TLR and Cell Death Inducing Chemotherapeutic and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a TLR agonist and an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell deatch inducing chemotherapeutic such as Doxorubcin or mitoxantone and the like, but not limited to those and a TLR agonist such as 0.1-10 mg/g R848 and the like, but not limited to those and 0.1-20 mg/g of an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those

### Combigels (TLR and Cell Death Inducing Chemotherapeutic and Wnt/β-Catenin Inhibitor

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a TLR agonist and an amount of a Wnt/6-catenin inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell deatch inducing chemotherapeutic such as Doxorubcin or mitoxantone and the like, but not limited to those and a TLR agonist such as 0.1-10 mg/g R848 and the like, but not limited to those and 0.1-20 mg/g of a Wnt/6-catenin inhibitor such as WAY-316606 or XAV939, and the like, but not limited to those

### Combigels (TLR and Cell Death Inducing Chemotherapeutic and Immune Checkpoint Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a TLR agonist and an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor ro Lymphocyte activating gene 3 (LAG3) inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a cell deatch inducing chemotherapeutic such as Doxorubcin or mitoxantone and the like, but not limited to those and 0.1-10 mg/g of a TLR agonist such as R848 and the like, but not limited to those and 0.1-20 mg/g of Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor such as BMS-8, INCMGA00012, CX072 or CCX4503 and the like, but not limited to those

### Combigels (TLR and TGFβ Signaling Inhibitor and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a TLR agonist and an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TGFß inhibitor such as RepSox or SD-208 and the like, but not limited to those and 0.1-10 mg/g of a TLR agonist such as R848 or Gardiquimod and the like, but not limited to those and 0.1-20 mg/g of an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those.

### Combigels (TLR and TGFβ Signaling Inhibitor and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a TLR agonist and an amount of a Wnt/β-catenin inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a TGFß inhibitor such as RepSox or SD-208 and the like, but not limited to those and 0.1-10 mg/g of a TLR agonist such as R848 or Gardiquimod and the like, but not limited to those and 0.1-20 mg/g of a Wnt/6-catenin inhibitor such as WAY-316606 or XAV939, and the like, but not limited to those

Combigels (TLR and TNFSFR agonist and Wnt/β-catenin inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount a TLR agonist and an amount of a Wnt/6-catenin inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of an TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those and 0.1-10 mg/g of a TLR agonist such as R848 or Gardiquimod and the like, but not limited to those and 0.1-20 mg/g of a Wnt/6-catenin inhibitor such as WAY-316606 or XAV939, and the like, but not limited to those

### Combigels (RIG-1 and Cell Death Inducing Chemotherapeutic and TGFß Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and a RIG-1 agonist and a TGFß inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains an amount of cell death inducing chemotherapeutic and a RIG-1 agonist and a TGFß inhibitor.

### Combigels (RIG-1 and Cell Death Inducing Chemotherapeutic and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a RIG-1 agonist and an amount of a OX40 (CD124) agonist, CD40 agonist, CD27 agonist, 4-1 BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of cell death inducing chemotherapeutic such as Doxorubcin or mitoxantone and 0.1-20 mg/g of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist such as DB36 or CVN, and the like, but not limited to those and 0.1-20 mg/g of a RIG-1 agonist such KIN1408, SLR14, MK4621, RGT100 or KIN1400 and the like, but not limited to those.

### Combigels (RIG-1 and Cell Death Inducing Chemotherapeutic and Wnt/3-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a RIG-1 agonist and an amount of a Wnt/6-catenin inhibitor.

In one embodiment, the gel forming composition such as the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Wnt/6-catenin inhibitor such as WAY-316606 or XAV939, and the like, but not limited to those and 0.1-20 mg/g of a RIG-1 agonist such as KIN1408, SLR14, MK4621, RGT100 or KIN1400 and the like, but not limited to those and contains 0.1-20 mg/g of a cell deatch inducing chemotherapeutic such as Doxorubcin or mitoxantone and the like, but not limited to those

### Combigels (RIG-1 and Cell Death Inducing Chemotherapeutic and Immune Checkpoint Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a RIG-1 agonist and an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor ro Lymphocyte activating gene 3 (LAG3) inhibitor.

In one embodiment, the gel forming composition such as LOIB 82.5% gel or Suben 60% gel or meLOIB 82.5% gel or nSuBen 60% gel or RaBen 60% gel or LacBen 60% gel or CLA-SuBen 40% gel or CLA-RaBen 40% gel or CLA-LacBen 40% gel contains 0.1-20 mg/g of a Wnt/6-catenin inhibitor such as WAY-316606, XAV939, and the like, but not limited to those and 0.1-20 mg/g of a RIG-1 agonist such as KIN1408, SLR14, MK4621, RGT100 or KIN1400 and the like, but not limited to those and contains 0.1-20 mg/g of a cell deatch inducing chemotherapeutic such as Doxorubcin or mitoxantone and the like, but not limited to those and containes 0.1-20 mg/g of Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor or Lymphocyte activating gene 3 (LAG3) inhibitor such as BMS-8, INCMGA00012, CX072 or CCX4503 and the like, but not limited to those

### Combigels (RIG-1 and TGFβ Signaling Inhibitor and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a RIG-1 agonist and an amount of a TGFβ signaling inhibitor and an amount of a OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

### Combigels (RIG-1 and TGFβ Signaling Inhibitor and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a RIG-1 agonist and an amount of a Wnt/β-catenin inhibitor.

### Combigels (RIG-1 and TNFSFR Agonist and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a RIG-1 agonist and an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of a Wnt/β-catenin inhibitor.

### Combigels (RIG-1 and TNFSFR Agonist and IDO Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a RIG-1 agonist and amounts of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of an IDO inhibitor.

### Combigels (RIG-1 and TGFβ Signaling Inhibitor and IDO Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a RIG-1 agonist and an amount of an IDO inhibitor.

### Combigels (RIG-1 and TGFβ Signaling Inhibitor and SHP2 Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a RIG-1 agonist and an amount of an SHP2 inhibitor.

### Combigels (STING and Cell Death Inducing Chemotherapeutic and TGFß Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and a STING agonist and a TGFß inhibitor

### Combigels (STING and Cell Death Inducing Chemotherapeutic and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a STING agonist and an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

### Combigels (STING and Cell Death Inducing Chemotherapeutic and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a STING agonist and an amount of a Wnt/β-catenin inhibitor.

### Combigels (STING and Cell Death Inducing Chemotherapeutic and Immune Checkpoint Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of cell death inducing chemotherapeutic and an amount a STING agonist and an amount of a Programmed death-ligand 1 and 2 (PD-L1 and PD-L2) inhibitor, programmed cell death protein 1 (PD1) inihibitor, PD-L1/PD-L1 checkpoint inhibitor, Anti-cytotoxic T-lymphocyte-associated protein-4 (anti-CTLA-4) inhibitor, T-cell immunoglobulin and mucin domain-3 (TIM-3) inhibitor ro Lymphocyte activating gene 3 (LAG3) inhibitor.

### Combigels (STING and TGFβ Signaling Inhibitor and TNFSFR Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a STING agonist and an amount of a TGFβ signaling inhibitor and an amount of a OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist.

### Combigels (STING and TGFβ Signaling Inhibitor and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a STING agonist and an amount of a Wnt/β-catenin inhibitor.

### Combigels (STING and TNFSFR Agonist and Wnt/β-Catenin Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a STING agonist and an amount of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of a Wnt/3-catenin inhibitor.

### Combigels (STING and TNFSFR Agonist and IDO Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a STING agonist and amounts of a TNFSFR agonist such as an OX40 (CD124) agonist, CD40 agonist, CD27 agonist, 4-1BB (CD137) agonist, Glucocorticoid-induced tumor necrosis factor receptor-related protein (GITR, CD357) agonist, inducible T-cell costimulator (ICOS) agonist or tumor necrosis factor related apoptosis-inducing ligand (TRAIL, CD253, TNFSF10) receptor agonist and an amount of an IDO inhibitor.

### Combigels (RIG-1 and TGFβ Signaling Inhibitor and IDO Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a STING agonist and an amount of an IDO inhibitor.

### Combigels (STING and TGFβ Signaling Inhibitor and SHP2 Inhibitor)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of a TGFβ signaling inhibitor and an amount a STING agonist and an amount of an SHP2 inhibitor.

### Combigels (Antibiotic and MMP Inhibitor and Interleukin Modulator)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of an inhibitor of matrix metalloproteinases (MMP) and an amount of an interleukin (IL-1, IL-6, IL-7, IL-8, IL-9, IL-10, IL-11, IL-12, IL-15, IL-17, IL-21, IL-22, IL-23, IL-27, IL-28, IL-29, IL-32, IL-33, IL-34, IL-35, IL-36 and IL-37) modulator.

### Combigels (Antibiotic and Osteoinductive or Osteogenic Drug and MMP Inhibitor

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of an osteoinductive or osteogenic drug and an amount of an inhibitor of matrix metalloproteinases (MMP).

### Combigels (Antibiotic and Hydroxy-3-Methylglutaryl Coenzyme a (HMG-CoA) Reductase Suppressants and Tyrosine Kinase Receptor Agonist)

In one embodiment, the gel forming composition such as LOIB gel or Suben gel or meLOIB gel or nSuBen gel or RaBen gel or LacBen gel or CLA-SuBen gel or CLA-RaBen gel or CLA-LacBen gel contains an amount of an antibiotic, or an anti-infectious effect agent and an amount of a hydroxy-3-methylglutaryl coenzyme A (HMG-CoA) reductase suppressants drug and an amount of a tyrosine kinase receptor agonist.

In one particular interesting embodiment the formulations of the present invention are combined with other therapies of cancer such as systemically administered chemotherapy, cell therapy such as T cell or NK cell therapy, or is combined with external beam radiotherapy or internal radiotherapy such as brachytherapy or is combined with cryo or hyperthermia therapy, or is combined with photodynamic therapy, or is combined with surgery.

### A Kit Comprising the Formulation

The present invention further comprises a kit comprising a syringe, a hypodermal needle adapted to the open end of said syringe, and a formulation as defined hereinabove. In one embodiment, the formulation is held in the interior or said syringe.

The gel forming system may be provided as a lyophilized powder, a suspension or a solution. Different components may be provided in one or more individual vials or pre-mixed in the interior or said syringe. Exemplary different components include, but are not limited to, the gel-forming system and the solid particles, and the formulation and one or more initiators.

The syringe may consist of a single, a multiple barrel syringe (e.g. MEDMIX SYSTEMS AG) or a double champer syringe (e.g. Debiotech S.A.) and the like, but not limited to those. Multiple barrel syringes and double champer syringes and the like may be useful for e.g. two components formulations were one component is a mixture of the gel forming system, the active pharmaceutical ingredient and potentially a contrast agent(s) and the other component is an initiator or salt suspension. In another embodiment a double chamber syringe may be useful where one chamber contains gel-forming component and the contrast agent(s) and the other chamber the active pharmaceutical ingredient(s).

The needle of the syringe can, in some embodiments, be one suitable for fine-needle biopsies. Non-limiting examples of syringes and needles for such embodiments are described in U.S. Pat. No. 7,871,383, U.S. patent publication No. 20040162505, and references cited therein. Such syringes and needles can advantageously be used in procedures where a biopsy of a tissue is to be taken in conjunction with imaging of the same, using a formulation of the invention. Preferably, the kit has a shelf-life of at least 6 months, such as at least 12 months when stored at, e.g., room temperature (typically 18 to 25° C.) or lower temperatures, such as, e.g., 2 to 10° C., such as about 5° C. The shelf-life can, for example, be determined as the period wherein the kit can be stored at 25° C., at 80% RH and 1 atm. pressure, and where the viscosity is kept within ±5% of the initial viscosity.

According to the present invention there is disclosed a composition being homogenous at 20 degrees Celsius, comprising at least:
(a) A hydrophobic disaccharide;
(b) A solvent selected from any of Dimethylsulfoxid, Ethanol (EtOH), Propylenecarbonate, or Benzyl Alcohol, or a combination thereof;
(c) A lipid oil;
(d) An iodinated hydrophobic lipid or iodinated saccharide; and
(e) At least one active pharmaceutically ingredient.

According to one embodiment of the present invention the composition comprises an iodinated hydrophobic lipid or an iodinated disaccharide with iodinated benzyl groups or iodinated benzoyl groups covalently bound.

According to yet another embodiment the composition comprises the iodinated hydrophobic lipid oil Lipiodol.

According to one specific embodiment of the present invention the composition comprises a hydrophobic iodinated lactose derivative.

According to one embodiment the composition comprises a disaccharide derivative selected from any of Lactose octabenzoate (LacBen), Sucrose octabenzoate (SuBen), Methyl hepta-O-isobutyryl-α,β-lactoside (MeLOIB), α,β-Lactose octa para-iodobenzoate, 3-iodobenzyl hepta-O-isobutyryl-α,β-lactoside (3-iodobenzyl LOIB), or a mixture hereof.

According to yet another embodiment of the present invention the composition comprises a lipid oil selected from any of glycerol trihexanoate, Glycerol trioctanoate (GTO), Glycerol tridecanoate, ethyl myristate, ethyl palmitate, ethyl oleoate, or mixtures thereof.

According to one embodiment the composition is a liquid before administration into a human or animal body and increases in viscosity by more than 2,000 centipoise (cP) after administration within 72 hours.

According to yet another embodiment the composition becomes a gel-like material or a solid after administration, such as a crystalline or amorphous solid and remains within 8 cm from the site of injection for at least 2 weeks.

According to another embodiment the composition increases in viscosity after administration into the human or animal body due to diffusion of the solvent out of the administered composition and into surrounding tissue.

According to one embodiment of the present invention at least 50 (w/w %) of the composition is a hydrophobic dissacharide.

According to another embodiment the composition further contains 0.1%-5% (w/w %) poly lactic acid (PLA) or poly(lactic-co-glycolic acid) (PLGA).

According to one specific embodiment of the present invention the solvent constitutes 4-16% (w/w %) of the composition.

According to another embodiment the gel composition is comprised of one of the following compositions: meLOIB:GTO:DMSO, SuBen:GTO:EtOH, LacBen:Lipiodol:EtOH, LacBen:Ethyl-palmitate:EtOH, CLA-8:SuBen:GTO:EtOH, and wherein one or more pharmaceutical ingredients are dissolved.

According to yet another embodiment the gel composition is comprised of one of the following compositions: meLOIB:GTO:DMSO (82.5:7.5:10 w/w), SuBen:GTO:EtOH (60:25:15 w/w), LacBen:Lipiodol:EtOH (60:25:15 w/w), LacBen:Ethyl-palmitate:EtOH (60:25:15), CLA-8:SuBen:GTO:EtOH (20:40:25:15), and wherein one or more active pharmaceutical ingredients are dissolved.

According to yet a further embodiment release of one or more active pharmaceutical ingredients after injection into the animal or human body is controlled by the hydrophobicity of the composition

According to one embodiment of the present invention the active pharmaceutical ingredients are chemically stable for more than 7 days at 20 degrees Celsius, preferably wherein less than 10% of the pharmaceutical ingredient changes chemical structure within 7 days at 20 degrees Celsius, more preferably less than 5% of the pharmaceutical ingredient changes chemical structure within 7 days at 20 degrees, and most preferably less than 1% of the pharmaceutical ingredient changes chemical structure within 7 days at 20 degrees.

According to one embodiment one or more of the active pharmaceutical ingredients is a drug that modulates an immune response.

According to another embodiment the active pharmaceutical ingredient is a TLR7 or TLR8 or a TLR7 and TLR8 agonist, or wherein the active pharmaceutical ingredient is a TLR7 or TLR8 or a TLR7 and TLR8 agonist and combined with at least one more active pharmaceutical ingredient that modulates an immune response.

According to one embodiment at least one active pharmaceutical ingredient selected from any of a class of TLR agonists, TGFβ inhibitors, IFN agonists, IDO inhibitors, GSK inhibitors, GSK inhibitors, RIG-I agonists, SHP inhibitors, SHP2 inhibitors, ICD inducers, Sting agonists, PD1 or PD-L1 inhibitors, CTLA4 inhibitors, OX40 agonist, CD40 agonist, CD137 agonist, GITR agonist, TNFSFR agonist or WNT/β-catenin inhibitors is dissolved.

According to another embodiment at least one active pharmaceutical ingredient is selected from any of the therapeutic agents Gardiquimod, Resiquimod (R848), Imiquimod, Repsox, Galunisertib, SD-208, NLG919, R08191, CHIR99021, PD0325901, TWS119, AR-A014418, SB415286, GSK-3 inhibitor-X, SHP099, PC-61275, Mitoxantrone, Bortezomib, Crizotinib, doxorubicin, XAV939, KIN1400, KIN1408, or Erythromycin, or a combination thereof.

According to the present invention there is also disclosed a composition wherein the active pharmaceutical ingredients are aTLR7 agonist and a TGFβ inhibitor.

According to the present invention there is also disclosed a composition wherein the active pharmaceutical ingredients are a RIG-1 agonist and a TGFβ inhibitor.

According to one embodiment the active pharmaceutical ingredients are a TLR7 agonist and a GSK inhibitor or a RIG-1 agonist and a GSK inhibitor.

According to the present invention there is also disclosed a composition wherein the active pharmaceutical ingredients are a TLR7 agonist and a WNT/β-catenin inhibitor or a RIG-1 agonist and a WNT/β-catenin inhibitor.

According to the present invention there is also disclosed a composition wherein the active pharmaceutical ingredients are a TLR7 agonist and a RIG-1 agonist.

According to one embodiment of the present invention there is disclosed a composition wherein the active pharmaceutical ingredients are a TLR7 agonist and a TNFSFR agonist or a RIG-1 agonist and a TNFSFR agonist.

According to yet another embodiment the active pharmaceutical ingredients are a TLR7 agonist and a SHP2 inhibitor or a RIG-1 agonist and a SHP2 inhibitor.

According to the one embodiment at least one of the active pharmaceutical ingredients is dissolved at a concentration higher than 1 mg/g of composition, preferably at least 2 mg/g of composition, more preferably at least 5 mg/g of composition, most preferably at least 10 mg/g composition at 20 degrees Celcius.

According to the present invention there is also disclosed a composition wherein the active pharmaceutical ingredients are a cytotoxic agent and one or more pharmaceutical ingredients that modulates an immune response.

According to the present invention there is also disclosed a composition wherein the active pharmaceutical ingredients are an ICD inducer and a TLR7 agonist or an ICD inducer and a RIG-1 agonist.

According to one embodiment of the present invention the active pharmaceutical ingredients are an ICD inducer and a TLR7 agonist and one immune modulating agent selected from any of TGFβ inhibitors, GSK inhibitors, SHP2 inhibitors, WNT/β-catenin inhibitors, OX40 agonist, CD40 agonist, CD137 agonist, GITR agonist, RIG-1 or TNFSFR agonist.

According to the present invention there is also disclosed a wherein the active pharmaceutical ingredients are an ICD inducer and a RIG-1 agonist and one immune modulating agent selected from any of TGFβ inhibitors, GSK inhibitors, SHP2 inhibitors, WNT/β-catenin inhibitors, OX40 agonist, CD40 agonist, CD137 agonist, GITR agonist or TNFSFR agonist.

According to one embodiment of the present invention the active pharmaceutical ingredients are released from the composition after administration to a human or animal body at comparable rates.

According to the present invention there is also disclosed a composition wherein the active pharmaceutical ingredients are released from the composition after administration to a human or animal body at substantially different rates.

According to one embodiment the composition comprises contrast agents that make the composition visible in one or more imaging modalities such as ultrasound imaging, CT imaging, x-ray imaging, fluoroscopy imaging, fluorescence imaging, MR imaging or OCT imaging.

According to the present invention there is also disclosed a method of administering a composition into diseased tissue of a human or animal body, wherein the composition is administered through a hypodermic needle, pig-tail catheter, intravascular catheter, endoscopy aspiration needle, bone marrow aspiration needle and a syringe, an endoscope, a bronchoscope, bone marrow injection device, or stereotactic injection frame under image guidance.

According to one specific embodiment a CT, fluoroscopy, ultrasound, OCT or x-ray image is recorded after administration to verify placement of the composition within the diseased tissue or intended tissue.

### FIGURE LEGENDS

FIG. 1: In vitro release of R848 from gels composed of SuBen or LOIB (see Example 2). A: The different gel formulations were prepared with a R848 concentration of 1.2 mg/mL. 100 μL of each R848 gel formulation was injected into 2 mL PBS and the cumulative release was evaluated over a period of 14 days. The cumulative release was calculated based on normalization to total drug release. Data are presented as mean±SD (n=2). B: The LOIB B gel formulation was prepared with R848 concentrations of 0.12 mg/mL, 0.24 mg/mL, 1.2 mg/mL, 2.4 mg/mL and 4.8 mg/mL. 50 μL of each R848 gel formulation was injected into 2 mL PBS and the cumulative release was evaluated over a period of 49 days. The cumulative release was calculated based on normalization to total drug release. Data are presented as mean±SD (n=2-3).

FIG. 2: In vivo release of R848 from the LOIB B gel formulation. The LOIB B gel formulation was prepared with R848 (1.2 mg/mL) and X-SAIB. Female BALB/c mice bearing subcutaneous CT26 tumors were treated with one intratumoral injection of R848 LOIB B gel therapy (3 mg/kg) containing X-SAIB. Mice were sacrificed at specified time points after injection: 1 hour, 3 hours, 24 hours, 3 days and 7 days. The remaining gel material was collected from the tumors and R848 and X-SAIB content was measured using UHPLC. For each time point, R848 release was calculated based on the R848/X-SAIB ratio in the collected gel compared to the total R848/X-SAIB ratio in the gel. A: Release over 7 days. Data are presented as mean±SD (n=5).

FIG. 3: Combination of RT and different R848 gel formulations (radioimmunotherapy). Female BALB/c mice were inoculated with CT26 tumor cells on day 0 and treated on day 12 (average tumor size: 110 mm³, n=6-8). A: Dosing schedules for the groups treated with five fractions of 2 Gy radiation as monotherapy or combined with different R848 gel formulations (3 mg/kg) administered intratumorally once per week for four weeks. Four different R848 gel formulations were tested: LAP, SuBen, LOIB A and LOIB B. For the groups receiving a R848 gel radioimmunotherapy, radiation was initiated four hours after the gel injection. A group receiving NT was included in the study as a control. B: Kaplan Meier survival plots for the individual groups. C: Mean tumor growth±SEM.

FIG. 4: Different dosing schedules combining RT and R848 LOIB B gel immunotherapy. Female BALB/c mice were inoculated with CT26 tumor cells on day 0 and treated on day 13 (average tumor size: 115 mm³, n=6-8). A: The different dosing schedules. Mice were treated with five fractions of 2 Gy radiation as monotherapy or combined with R848 LOIB B gel immunotherapy. Gel therapy was administered one or four hours prior to the first dose of radiation, 24 hours after the first dose of radiation or 72 hours after the last dose of radiation. R848 gel immunotherapy (3 mg/kg) was administered intratumorally once per week for four weeks. A group receiving NT was included in the study as a control. B: Kaplan Meier survival plot of individual groups. C: Mean tumor growth±SEM.

FIG. 5: Combination of RT and escalating doses of R848 LOIB B gel therapy. Female BALB/c mice were inoculated with CT26 tumor cells on day 0 and treated on day 12 (average tumor size: 120 mm³, n=8). A: Dosing schedules for the groups treated with five fractions of 2 Gy radiation as monotherapy or combined with the LOIB B gel formulation containing R848. 25 μL or 50 μL R848 gel therapy was administered intratumorally as weekly doses of 3 mg/kg for a total of four treatments, biweekly doses of 6 mg/kg for a total of two treatments or one dose of 12 mg/kg. For the groups receiving a R848 gel radioimmunotherapy, radiation was initiated one hour after the gel injection. A group receiving NT was included in the study as a control. B: Kaplan Meier survival plot of the groups. C: Mean tumor growth±SEM.

FIG. 6: IL-6 cytokine levels in mouse plasma. Female BALB/c mice were inoculated with CT26 tumor cells on day 0 and treated on day 12 (average tumor size: 130 mm³, n=4). The mice were treated with 2 Gy radiation as monotherapy or in combination with the LOIB B gel formulation (radioimmunotherapy) containing 3 mg/kg, 6 mg/kg or 12 mg/kg R848. The R848 gel immunotherapy was administered intratumorally in volumes of 25 μL or 50 μL. For the groups receiving a combination of RT and R848 gel therapy, the radiation was initiated one hour after the gel injection. A group receiving NT was included in the study as a control. IL-6 measurements were conducted by ELISA. A: IL-6 levels in plasma after treatment. B: Tumor volumes one day prior to treatment. Data are presented as mean±SD. A one-way ANOVA was performed followed by a Tukey's multiple comparisons test. A significant difference in mean is indicated with * (p<0.05) or ** (p<0.01).

FIG. 7: Radiographic contrast was provided by the inclusion of 15% and 25% Lipiodol (iodinated lipids) in the gel formulation. The inclusion of Lipiodol provided high radiographic contrast across all scans performed 10 minutes (scan 1), 30 minutes (scan 2), 2 hours (scan 3) and two months (scan 4) after the injection of the gel formulation containing Lipiodol. The injected formulation displayed the expected condensation following solvent diffusion, this was demonstrated by increased radiographic contrast and decreased volume, which further demonstrates that iodinated lipids are retained in the formulation. A: Mean and maximum marker radiographic contrast during scans 1-4, B: Marker volume on scans 1˜4 and C: Axial CT scan slices from scan 1 to 4 (left to right) demonstrating that the gel can be clearly identified based on radiographic contrast both with inclusion of 15% (top row) and 25% (bottom row) of iodinated lipids. Images are from randomly selected regions and as such does not provide information in positional stability.

FIG. 8: LOIB (upper) and SuBen (lower) based gels are transparent, homogeneous and form spherical gels upon injection.

FIG. 9: R848 release kinetics from injected gels can be controlled by the concentration of Lactose octa isobutyrate (LOIB) and glycerol trioctanoate (GTO) in the gel. 1.5 mg R848 per gram gel was solubilized in the gels and 100 μl of each gel was injected in duplicates into 2 ml of PBS. Samples were taken at fixed time points and the amount of released R848 was measured using fluorescence spectrometry.

FIG. 10. In vitro release of R848—effect of PLA. The addition of poly lactic acid (PLA) Mw 8-18 kDa to the LOIB gels can reduce burst and total release of R848 from injected gels. 0.5% of PLA was complemented to a 79.5% (% w/w)) LOIB formulation and R848 release was compared with a 80% (% w/w) LOIB formulation. 1.5 mg R848/gram gel was solubilized in the gels and 100 μl of each gel was injected in duplicates into 2 ml of PBS. Samples were taken at fixed time points and the amount of released R848 was measured fluorescence spectrometry.

FIG. 11: R848 cumulative release in percent from gels injected into PBS is not dependent on R848 concentrations.

FIG. 12: In vitro release of R848—effect of solvent. R848 release from injected gels can be controlled by the concentration of Sucrose benzoate (SuBen) and the addition of poly lactic acid (PLA) Mw 8-18 kDa as well as changing EtOH to BnOH. The release of R848 from four different SuBen-based formulations was investigated as indicated in the graf. 1.5 mg/gram gel of R848 was included in the gels and 100 μl of each gel was injected into 2 ml of PBS. Samples were taken at fixed time points and the amount of released R848 was measured by fluorescence spectrometry (all percentages are (% w/w)).

FIG. 13: The TGFβ signaling inhibitors RepSox and Galunisertib can be released in a controlled manner from LOIB-based gels. 8 mg/gram gel of RepSox or Galunisertib was solubilized in 82.5 (% w/w) LOIB gels. 100 μl of each gel was injected in duplicates into 2 ml of PBS. Samples were taken at fixed time points and the amount of released TGFβ inhibitors was measured using uv spectroscopy (all percentages are (% w/w)).

FIG. 14: The TGFβ inhibitor RepSox can be dissolved together with R848 in LOIB-based gels.

FIG. 15: Comparison of intratumoral R848 gel radioimmunotherapy and multidrug intratumoral R848 and TGFbi gel radioimmunotherapy in CT26 tumors on Balb/C mice. Female BALB/c mice were inoculated with CT26 tumor cells on day 0 and treated on day 14 (average tumor size: 110 mm³). A: Dosing schedules for the groups treated with three fractions of 2 Gy radiation as monotherapy (RT) or combined with the LOIB gel containing R848 or R848 and TGFbi (Galunisertib or RepSox) or SuBen gel containing R848 and TGFbi (SD-208). Gel therapy was administered intratumorally as weekly doses (3 mg/kg R848 and 20 mg/kg TGFbi) for a total of four treatments. For the groups receiving a R848 gel radioimmunotherapy, radiation was initiated one hour after the gel injection. Group receiving no treatment (NT) was included as controls. B: Mean tumor growth curves. C: Kaplan Meier survival plot.

FIG. 16: Adoptive T lymphocyte therapy. Combined R848 LOIB or R848+TGFßi LOIB gel and adoptive T lymphocyte therapy using OVA specific OT.1 T lymphocyte (10⁶/mouse) in C57BL/6 mice bearing well established EG7.OVA tumors. A: Kaplan Meier survival plot of study including groups receiving R848 LOIB or R848+TGßi LOIB gel as single administrations with or without OVA specific OT.1 T lymphocytes including OVA specific OT.1 T lymphocyte only group and untreated controls. B: Kaplan Meier survival plot of study including groups receiving R848 LOIB or R848+TGFbi LOIB gel as two administrations placed 7 days apart with or without OVA specific OT.1 T lymphocytes and including OVA specific OT.1 T lymphocyte only group and untreated controls (The Kaplan Meier plots A and B are from the same study and split into two graphs for improving overview of data). C: Median tumor volume through the study period for the included groups. TGFßi (RepSox): 20 mg/kg, R848 3 mg/kg.

FIG. 17: Example of a completely dissolved homogeneous gel (left vial), a semi-dissolved gel (middle) and a precipitated gel (right).

FIG. 18. Examples of APIs formulated in carbohydrate ester gels. From left, XAV939, KIN-1400, KIN1408, CHIR99021, TWS119 and AR-A014418.

FIG. 19. The CT contrast agents CLA-1 and CLA-8 can be incorporated into SuBen and LOIB gels. From the left (GEL 1-3), 5, 10 and 20% of CLA-8 solubilized in SuBen-based gels, respectively. GEL 4, LOIB gel containing 10% of CLA-8. GEL 5, CLA-1 gel containing 82.5% of CLA-1.

FIG. 20: CLA-8 stability in gel.—A: Chromatogram of Gel 3 after 3 weeks incubated at 37° C. The retention time of SuBen is 10.3 min and the retention time of CLA-8 is 12.8 min (UV detection at 280 nm). B: Chromatogram of Gel 4 after 3 weeks incubated at 37° C. The retention time of CLA-8 is 12.8 min (UV detection at 280 nm).

FIG. 21: R848 and gardiquimod undergo transesterification in LOIB gels. A: HPLC chromatograms comparison between a R848 LOIB gel and a LOIB gel blank (without drug) after 7 days at 37° C. (detection at 320 nm). B: HPLC chromatograms comparison between release media from a gardiquimod LOIB gel and a LOIB gel blank (without drug) after 14 days at 37° C. (detection at 320 nm). C: Chemical structures, mass and m/z values of R848 and R848-isobutyrate (R848-IBA), both found in R848 LOIB gels. D: Percentage of R848-IBA found in the R848 LOIB gels over 7 days as calculated from the total AUC at 320 nm. Data are presented as mean±SD (n=2).

FIG. 22: Effect of temperature and pH on the transesterification of R848 in LOIB gels. A: Percentage of R848-IBA in 1.2 mg/mL LOIB gels after 5 days at either 4° C. or 37° C. as calculated from the total AUC at 320 nm. B: Percentage of R848-IBA in 1.2 mg/mL LOIB gels containing either BA, TEA or none at 37° C., as calculated from the total AUC at 320 nm.

FIG. 23: Chromatograms and suspected modification of R848 in LOIB- and SAIB based gels. A: Comparison of HPLC chromatograms of SAIB:GTO:EtOH (82.5:7.5:10 (% w/w)), LOP:GTO:EtOH (82.5:7.5:10 (% w/w)) and LOIB:GTO:EtOH (82.5:7.5:10 (% w/w)) release media from gels after 7 days at 37° C. Peak 1 corresponds to native R848, while peaks 2 and 3 represent the modified versions of R848 with a mass of 370.20 and 384.22, respectively (UV detection at 320 nm). B: Chemical structure and mass of the suspected modified versions of R848, which form in LOIB- and SAIB based gels (R848-IBA), as well as in LOP-based gels (R848-PROP). The LC-MS results correspond to the analysis of the dissolved gels after 14 days at 37° C.

FIG. 24: A Chromatogram of R848 in meLOIB:GTO:EtOH (82.5:7.5:10 (% w/w)) gel. The gel contained 1 mg/g Resiquimod and the chromatogram was recorded after 2 months at 37° C. The retention time of Resiquimod is 7.8 min; UV detection was done at 320 nm.

FIG. 25: In vitro release of Gardiquimod and Resiquimod from gels. (A) Cumulative release of Gardiquimod is compared for SuBen:GTO:EtOH and SuBen:Lipiodiol:EtOH gels. (B) Cumulative release of Resiquimod is compared for SuBen:GTO:EtOH, SuBen:Ethyl-palmitate:EtOH gels and SuBen:Ethyl-oleate:EtOH gels.

FIG. 26: Viscosity of gels. The viscosity is shown as function of time (solvent efflux) for SuBen gels (A) and LOIB gels (B). The LOIB gels contained either EtOH, DMSO, BnOH, PC, or acetone as solvent. The SuBen gels contained either EtOH, BnOH or PC as solvent. The viscosities were determined in doublets on an EMS-1000 viscometer.

FIG. 27: Pharmacokinetics and biodistribution of tritiated R848. Pharmacokinetics and biodistribution was evaluated after free intratumoral (IT) or intravenous (IV) injection of free R848 and intratumoral injection of R848 in LOIB:GTO:EtOH (82.5:7.5:10 (% w/w)) gel (GEL). Results are expressed as % of injected dose per gram in the specified tissue (% ID/g) at multiple time points after injection.

FIG. 28: In vivo API release profiles are shown for Galunisertib, RepSox, and SD-208. The API release is presented as mean±SEM (n=4).

FIG. 29: Gel formulations of doxorubicin; DOX-HCL and DOX base. Formulations of doxorubicin. A: 0.8 mg/mL DOX base in SuBen:GTO:EtOH (60:25:15) (left) and 0.8 mg/g DOX-HCl in SuBen:GTO:EtOH (60:25:15 (% w/w)) (right). B: 0.8 mg/g DOX base LOIB:GTO:EtOH (82.5:7.5:10 (% w/w)) (left) and 0.8 mg/mL DOX HCl LOIB:GTO:EtOH (82.5:7.5:10 (% w/w)) (right).

FIG. 30: In-vitro release assay for DOX base in SuBen and LOIB gel. In-vitro release assay. A: 50 μL injected from 0.8 mg/g DOX base in LOIB:GTO:EtOH (82.5:7.5:10 (% w/w)) (left) and 0.8 mg/g DOX base in SuBen:GTO:EtOH (60:25:15 (% w/w)) (right) in PBS. B: Fluorescence measurements at TO and 7 days after incubation at 37° C. TO measurements correspond to PBS (blank release media). Data is shown as mean±SEM (n=2).

FIG. 31: Therapeutic evaluation of chemo-immunotherapy gel. Chemo-immunotherapy gel providing an intratumoral release of chemotherapy (doxorubicin or mitoxantrone) and immune activating R848 and RepSox in CT26 tumors on Balb/C mice. A total of three intratumoral injections were performed at 7 days interval. A: Tumor growth curves (mean±SEM). B: KaplanMeier survival plot. C: Bodyweight through the study period (mean±SEM).

FIG. 32: Visibility on CT imaging of SuBen:Lipiodol:EtOH gel in soft tissue and bone. (A) CT scans of SuBen:Lipiodol:EtOH (60:25:15 (% w/w)) gel injected in the musculature around the tibia and fibula (white arrows) (100 μL SuBen:Lipiodol:EtOH (60:25:15 (% w/w)) gel, 21G/76 mm needle 1 ml syringe). (B) CT scan of SuBen:Lipiodol:EtOH (60:25:15 (% w/w)) gel injected in a intraosseous cavity (white arrows) generated by sharp curvetting and in the trabecular bone marrow surrounding the excavated cavity (black arrows) and peri-osseous (white arrow head). All injections performed using a 21G/76 mm needle 1 ml syringe. Intraosseous injections were performed through 2 mm holes drilled in the cortical bone.

FIG. 33: Carbohydrates may be fully or partly acylated with one or more iodine containing groups. The carbohydrates may be fully or partly acylated with one or more of the groups shown. R=iodine containing aromatic acyl groups or aliphatic or aromatic acyl groups such as acetate, propionate, butyrate, isobutyrate, pivaloate, valerate, isovalerate, hexanoate, benzoate, PABA acylation or PEG acylation (where n=1-100) or OH.

FIG. 34: Carbohydrates may be functionalized with aromatic iodine containing aromatic acyl groups. The carbohydrates may be functionalized with aromatic iodine containing aromatic acyl groups (R₂) selectively on primary alcohols and/or amines on one or more positions in any acylation pattern, the rest of the positions may be functionalized with one or more aliphatic or aromatic acyl groups (R) in any acylation pattern. R=aliphatic or aromatic acyl groups such as acetate, propionate, butyrate, isobutyrate, pivaloate, valerate, isovalerate, hexanoate, benzoate, PABA acylation or PEG acylation (where n=1-100). R₂=aromatic iodine containing acyl groups with different substitution patterns.

FIG. 35: The anomeric center of the carbohydrates may be protected with aromatic iodine containing ethers (R₃). The primary positions and/or amines may be functionalized with aromatic iodine containing acyl groups (R₂) on one or more positions. The rest of the positions may be functionalized with one or more of the acyl groups denoted “R” in any pattern. R=aliphatic or aromatic acyl groups such as acetate, propionate, butyrate, isobutyrate, pivaloate, valerate, isovalerate, hexanoate, benzoate, PABA acylation or PEG acylation (where n=1-100). R₂=aromatic iodine containing acyl groups with different substitution patterns. R₃=aromatic iodine containing ether groups with different substitution patterns.

FIG. 36: The anomeric center of the carbohydrates may be protected with aliphatic linear or branched ethers or aromatic ethers of different length (R₃). The primary positions and/or amines may simultaneously be functionalized with aromatic iodine containing acyl groups (R₂) on one or more positions, the rest of the positions may be functionalized with one or more of the acyl groups denoted “R” in any pattern. R=aliphatic or aromatic acyl groups such as acetate, propionate, butyrate, isobutyrate, pivaloate, valerate, isovalerate, hexanoate, benzoate, PABA acylation or PEG acylation (where n=1-100). R₂=aromatic iodine containing acyl groups with different substitution patterns. R₃=aliphatic linear or branched ethers (where n=1-20) or aromatic ether groups.

FIG. 37: The anomeric center of the carbohydrates may be protected with aliphatic linear or branched ethers or aromatic ethers of different length (R₂). The rest of the positions may be functionalized with one or more of the acyl groups denoted “R” in any pattern. R=aliphatic or aromatic acyl groups such as acetate, propionate, butyrate, isobutyrate, pivaloate, valerate, isovalerate, hexanoate, benzoate, PABA acylation or PEG acylation (where n=1-100). R₂=aliphatic linear or branched ethers (where n=1-20) or aromatic ether groups.

### EXAMPLES

### Example 1: Log P Analysis of Gel Forming Compounds

The gels of the current disclosure are composed of hydrophobic solvents, co-solvents and esterified carbohydrates that all have differing hydrophobicity. The physiochemical properties of the gel allow for solubilization and sustained release of hydrophobic (log P>0) compounds. The hydrophobicity of the individual gel compounds can be quantified by the oil-water partitioning coefficients which is given by the Log P value. In the present example, Log P values were obtained by calculations based on the algorithm of Viswanadhan et 25 al (Viswanadhan, V. N.; Ghose, A. K.; Revankar, G. R.; Robins, R. K., J. Chem. Inf. Comput. Sci., 1989, 29, 163-172). The log P value can also be determined by octanol-water partitioning experiment. Positive log P values are characteristic hydrophobic compounds, whereas negative log P values indicate a hydrophilic compound. Log P values have been computed for the most relevant compounds of this disclosure, and are presented in table 1.

TABLE 1
LogP values for solvents, oils (co-solvents),
carbohydrate esters and drug compounds.

| Compounds | logP | Compounds | LogP |
|---|---|---|---|
| Solvents | | TGFβ inhibitors | |
| Dimethylsulfoxid (DMSO) | −1.40 | Repsox | 2.50 |
| Ethanol (EtOH) | −0.16 | Galunisertib | 2.64 |
| Propylenecarbonate (PC) | 0.79 | SD-208 | 3.50 |
| Benzyl Alcohol (BnOH or BA) | 1.21 | | |
| | | IDO inhibitors | |
| Co-solvents | | NLG919 | 3.00 |
| Glycerol trihexanoate (GTH) | 5.59 | | |
| Glycerol trioctanoate (GTO) | 8.25 | IFN-agonist | |
| Glycerol tridecanoate (GTD) | 10.92 | RO8191 | 2.46 |
| Carbohydrate esters | | GSK inhibitors | |
| Lactose octaacetate (LOA) | −1.17 | CHIR99021 | 4.06 |
| Lactose octapropionate (LOP) | 4.43 | PD0325901 | 3.98 |
| Sucrose acetate isobutyrate | 6.46 | TWS119 | 3.05 |
| (SAIB) | | | |
| Lactose octaisobutyrate (LOIB) | 8.77 | SB415286 | 2.19 |
| Sucrose octabenzoate (SuBen) | 15.30 | GSK-3 inhibitor-X | 4.24 |
| TLR agonists | | SHP2 inhibitors | |
| Gardiquimod | 1.40 | SHP099 | 2.74 |
| Resiquimod (R848) | 1.72 | PC-61275 | 6.85 |
| Imiquimod | 2.65 | | |
| | | ICD inducers | |
| | | Mitoxantrone | 0.65 |
| | | Doxorubicin | 1.27 |
| | | Bortezomib | 1.53 |
| | | Crizotinib | 3.57 |

### Discussion

In table 1, the solvents have log P values in the range −1.40 to 1.21 and are thereby soluble in both the hydrophobic gel phase and the aqueous phase. Upon exposure of the gel towards water in buffers or in tissues, the solvent may diffuse out of the gels and cause a non-solvent induced phase separation (NIPS) where the viscosity of the injected gel solution increases or the gel even solidifies.

The co-solvent examples presented in table 1 are significantly more hydrophobic than the solvents, and have log P values in the range 5.59 to 10.92. The co-solvent thus have negligible solubility in water, and therefore remain in the gel carbohydrate ester solution that remain after solvent efflux. In the carbohydrate ester solution, the co-solvents reduce the viscosity and increases diffusion and can therefore be used for tuning of the drug compound release.

The carbohydrate esters presented in table 1 have log P values in the range −1.17 to 15.30. The LAP mixture comprising 50% LOA and 50% LOP is only partially hydrophobic, whereas increasing hydrophobicity is observed for SuBen>LOIB>SAIB. For some compounds, improved solubility has been observed for SuBen gels compared to LOIB gels suggesting that the higher hydrophobicity of SuBen can enhance the drug compound solubility.

The drug compounds have Log P values ranging from 1.40 to 6.86. Compounds with the lowest log P have the weakest hydrophobic (van der Waals) interactions with the gel constituents and further have the highest solubility in the aqueous phase. Higher and more rapid release of compounds with lower log P values have been observed for e.g. Resiquimod and Imiquimod, where the latter is retained to a larger extend.

### Conclusion

Solvents with log P values in the range −1.40 to 1.21 are appropriate for inducing non-solvent induced phase separation upon exposure of the gels to water (either in buffer or interstitial tissue fluids). The co-solvents have log P values in the range 5.59 to 10.92, and remain in the gel depot after NIPS where they act to control the viscosity and diffusion of drug compounds subsequently. The carbohydrate esters have log P in the range 0 to 15.30 and provide different drug solubility and retention depending on their hydrophobicity. Optimal release and solubility of drug substances in the gels are obtained for compounds which have log Ps in the range log P>1 and log P<5.

### Example 2: Preparation of LAP, SuBen, LOIB A and LOIB B Gels

The aim of the current example is to explain the methods for preparation of gel compositions. Gels comprising LAP, SuBen and LOIB were prepared with composition given in table 2.

TABLE 2
Compositions of R848 gel formulations. Weight
ratio (w/w %) of carbohydrates, solvents and
other additives in the gel formulations.

| | Chemicals | | | | | | | |
|---|---|---|---|---|---|---|---|---|
| Gel | LOIB | SuBen | LAP | PLGA | PLA | GTO | EtOH | PC |
| LAP | | | 68 | 2 | | 15 | 5 | 10 |
| SuBen | | 60 | | | 0.5 | 25 | 15 | |
| LOIB A | 80 | | | | | 10 | 10 | |
| LOIB B | 82.5 | | | | | 7.5 | 10 | |

Abbreviations: EtOH = ethanol, GTO = glycerol trioctanoate, LAP = lactose acetate:lactose propionate 1:1, LOIB = lactose octa iso butyrate, PC = propylene carbonate, PLA = poly lactic acid, PLGA = poly(lactic-co-glycolic acid), SuBen = sucrose benzoate.

### Method:

The LAP-based gel formulation was prepared by weighing lactose acetate, lactose propionate (1:1) and poly(lactic-co-glycolic acid) (PLGA) (lactide:glycolide 75:25, MW 4-15 kDa) into glass vials and adding a volume of R848 solubilized in tert-Butanol (t-BuOH):water (9:1) resulting in a concentration of 1.2 μg/μl R848 in the finished gel formulation. Next the solutions were freeze-dried overnight. On the day of treatment, glycerol trioctanoate (GTO), ethanol (EtOH) and propylene carbonate (PC) were added to the freeze-dried powder to generate a final gel-forming matrix of LAP:PLGA:EtOH:PC:GTO with the weight ratio 68:2:5:10:15. In order for the gel to become a homogenous solution, it was placed in an ultrasonicator (USC200TH, VWR Collection) at 60-80° C. with occasional vortexing before treatment.

For the SuBen- and LOIB-based gel formulations, R848 solubilized in t-BuOH:water (9:1) was weighed into glass vials and freeze-dried. Next, poly lactic acid (PLA) (MW 10-18 kDa), LOIB and SuBen were solubilized in GTO and EtOH in the ratio and amount required to generate gel-forming matrixes of SuBen:SuBen:PLA:GTO:EtOH (60:0.5:25:15), LOIB A:LOIB:GTO:EtOH (80:10:10) and LOIB B:LOIB:GTO:EtOH (82.5:7.5:10). The weight ratios (w/w %) are listed in the parentheses. Subsequently, the solutions were prepared with R848 concentrations ranging from 0.12 mg/mL-4.8 mg/mL. Next, the gel formulations were placed in an ultrasonicator (USC200TH, VWR Collection) at 60-80° C. for 1-2 hours to generate homogenous solutions that were subsequently stored on room temperature until use.

Lactose propionate was produced by custom synthesis. LOIB was purchased from Carbosynth, R848 was purchased from Ark Pharm and all other reagents were purchased from Sigma-Aldrich.

### Results and Discussion:

Homogeneous gels based on LAP, LOIB and SuBen were produced containing various amounts of R848. Simple mixing of the compounds followed by heating and sonication was sufficient for producing transparent gel formulations.

### Conclusion

Homogeneous and transparent gel formulation containing R848 were produced by simple mixing of compounds followed by heating and sonication.

### Example 3: In Vitro Release of R848 from LOIB and SuBen Gels

The aim of the current example is to investigate the release of R848 from LOIB and SuBen-based gels.

### Methods:

The gel formulations were prepared as described in example 1. 50 μL or 100 μL gel formulation was injected into 2 mL phosphate buffered saline (PBS) in a glass vial and incubated at 37° C. For each gel formulation tested, duplicates or triplicates were prepared. At fixed time points, aliquots of 1 mL were removed and replaced with 1 mL PBS. R848 content in the aliquoted samples was measured by fluorescence spectroscopy (fixed lambda assay, excitation: 330 nm, emission: 355 nm) on a microplate reader (Spark, Tecan). The cumulative release of R848 from the gel formulations was calculated by normalization to total amount of R848 in the gel.

### Results and Discussion

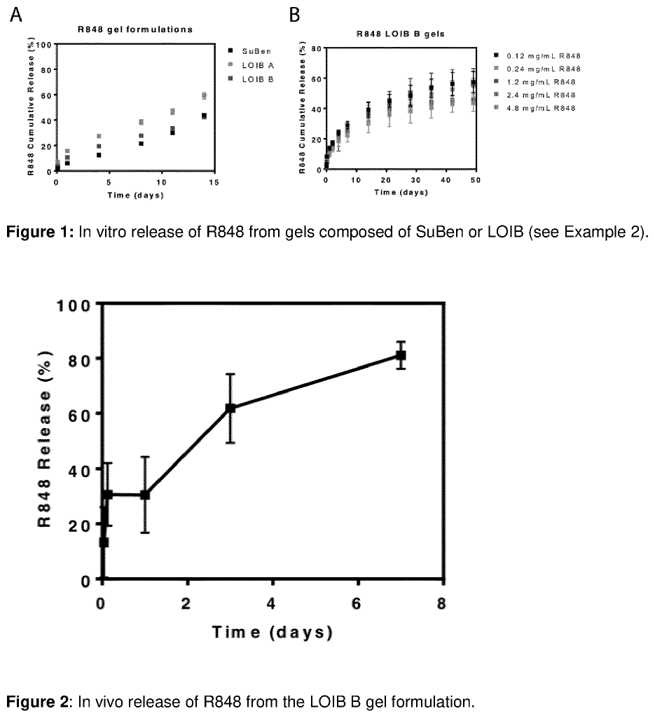

The in vitro release of R848 from gels was evaluated by injection of these into PBS buffer following evaluation of the release media using fluorescence spectroscopy. Cumulative release from SuBen and LOIB gels was compared, and the impact of varying R848 content was investigated for the LOIB B gel formulation (LOIB:GTO:EtOH 82.5:7.5:10 (% w/w)), presented in example 2). The obtained release results are compiled in FIG. 1 and are presented as cumulative release in percent.

The SuBen gel was found to have the lowest release rate followed by LOIB B and LOIB A having the highest release rate (FIG. 1A). Cumulative releases of 40-60% were obtained after 14 days for the three gel compositions. The influence of varying R848 concentration on the cumulative release from a LOIB B gel formulation was investigated, and the relative cumulative release was found to be independent of the R848 concentration, i.e. similar percentage of R848 was released independently of the actual R848 gel content. The absolute release of R848 therefore scales with the R848 concentration in the initial gel.

### Conclusion

The SuBen based gel was found to have the lowest release, and the relative cumulative release from a LOIB B gel was found to be independent of the initial R848 gel concentration.

### Example 4: In Vivo Release of R848 from LOIB Gels

The current example demonstrates the release of R848 from LOIB gels upon intratumoral administration.

### Method:

6-8 week old female BALB/c mice were inoculated with 3×10⁵ CT26 tumor cells in 100 μl of RPMI on the right flank by subcutaneous injection on day 0. When the average tumor size reached approximately 500 mm³ mice were divided into five groups based on a size-dependent randomization (n=5 per group). Mice were treated with one intratumoral injection of 50 μL LOIB gel formulation (LOIB:xSAIB:GTO:EtOH (77.5:5:7.5:10 (% w/w)) containing R848 (1.2 mg/mL, 3 mg/kg) and 5% xSAIB as a reference. The groups of mice were sacrificed at specified time points after injection (1 hour, 3 hours, 24 hours, 3 days and 7 days) and the remaining gel material in the tumor was collected. The collected gel and gel that had not been injected (for reference) was dissolved in 200 μl acetonitrile (MeCN) with 1% trifluoroacetic acid (TFA) and the samples were subsequently filtered through 0.45 μm pore syringe filters. R848 and xSAIB content was measured using an UHPLC instrument (Shimadzu) with ultraviolet to visible radiation detection at 190-800 nm. Samples were injected (10 μL) onto an XTerra C8 column (2017, 5 μm, 4.6×150 mm, Waters; temperature 40° C.). The separation method used a gradient that consisted of mobile phase A (5% MeCN, 0.1% TFA in water) and mobile phase B (0.1% TFA in MeCN) with 0-100% phase B over a period of 15 minutes. The UHPLC data was correlated to a standard curve and the R848 release was calculated based on the R848/xSAIB ratio in the collected gel compared to the total R848/xSAIB ratio in the gel.

### Results and Discussion

Gel containing R848 was intratumorally administered in CT26 tumor bearing BALB/c mice. Gels were collected as function of time and the remaining R848 was determined by UPLC. The release in percent is presented in FIG. 2.

Upon intratumoral administration, the LOIB gel displayed an initial burst release followed by continuous release. Approximately 80% of the R848 was released at day 7.

### Conclusion

LOIB:xSAIB:GTO:EtOH (77.5:5:7.5:10 (% w/w)) gels provide continuous and sustained release of R848 for the 7 days evaluated after intratumoral administration of the gel depot.

### Example 5: Therapeutic Efficacy of LAP, SuBen and LOIB Type R848 Gels in Combination with External Beam Radiotherapy (RT)

The aim of the current example was to investigate the efficacy of LAP, SuBen and LOIB type gels containing R848 in combination with RT (radioimmunotherapy). The composition of the tested gels is given in table 2.

### Methods:

The gels were prepared as described in example 2. 6-7-week-old female BALB/c mice were inoculated with 3×10⁵ CT26 tumor cells in 100 μl of RPMI on the right flank by subcutaneous injection on day 0. Tumors were allowed to grow until their average size exceeded 100 mm³ before the mice were divided into treatment groups based on a size-dependent randomization (n=6-8 per group). For each experiment, the time point and average tumor size at treatment initiation is indicated in the figure legend. Mice were treated with R848 gel therapy administered as weekly doses of 3 mg/kg for a total of four treatments. The R848 gel therapy was administered by intratumoral injections of 50 μL with mice anesthetized by inhalation anesthesia (˜4.5% sevoflurane). Radiation was delivered in five fractions of 2 Gy. Prior to radiation, the mice were anesthetized (˜4.5% sevoflurane) and fixated with a shielding device only exposing the right tumor bearing leg. Radiation was delivered with a dose rate of 1 Gy/minute (12.5 mAs/320 kV) using a dedicated small animal irradiation device (X-RAD 320, Precision X-Ray, Inc.). The R848 gels were administered one hour prior to RT. Both tumor and bodyweight measurements were collected 2-3 times per week. Mice were terminated from the study once their tumors reached a tumor volume ≥1000. Furthermore, study endpoints included weight loss >15% and presence of extensive ulcerations on the tumors. The tumors were measured by a digital caliper and the tumor volumes were calculated using the equation (length×width2)/2. Mice that survived more than 100 days post inoculation were defined as long-term survivors.

### Results and Discussion

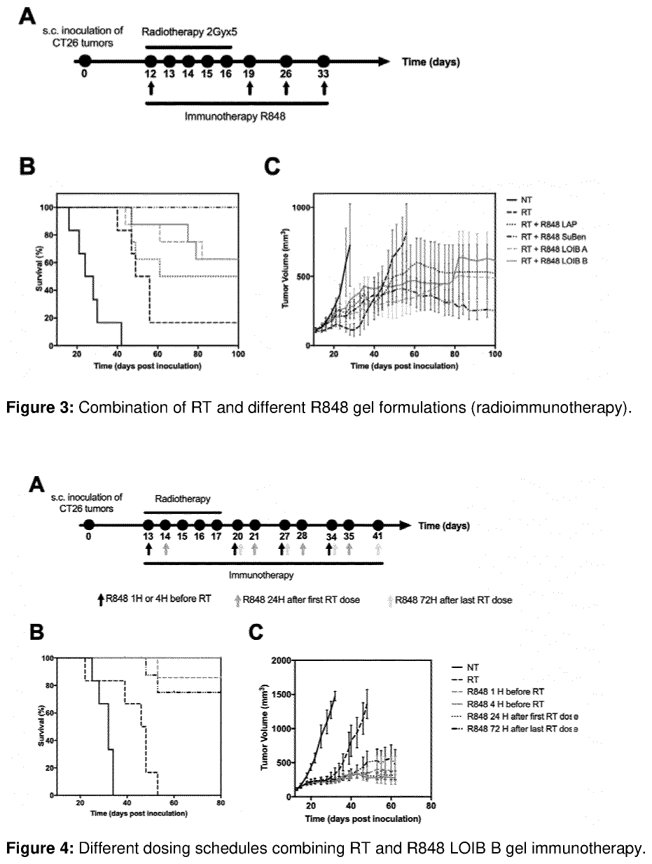

Four different R848 gel formulations with compositions given in table 2 were investigated and the dosing schedule and results are compiled in FIG. 3 and in table 3.

TABLE 3
Overview of groups, treatments and results from
the efficacy study evaluating the combination
of RT and different R848 gel formulations.

| | Parameters | | | |
|---|---|---|---|---|
| | | | Median | Long-term |
| | Treatment | Treatment | survival | survivors |
| Group | RT | R848 | [days] | [%] |
| NT | — | — | 30 | 0 |
| RT | 5 × 2 Gy | — | 56 | 25 |
| RT + R848 LAP | 5 × 2 Gy | 4 × 3 mg/kg | >100 | 57 |
| RT + R848 SuBen | 5 × 2 Gy | 4 × 3 mg/kg | >100 | 100 |
| RT + R848 LOIB A | 5 × 2 Gy | 4 × 3 mg/kg | >100 | 71 |
| RT + R848 LOIB B | 5 × 2 Gy | 4 × 3 mg/kg | >100 | 63 |

Radioimmunotherapy using the LAP, SuBen, LOIB A and LOIB B R848 releasing gel formulations displayed impressive tumor control and more than half of all mice displayed complete rejection of CT26 tumors. The highest percentage of complete responders was obtained for the SuBen gel, followed by the LOIB A, LOIB B and LAP gel formulations. Median survival exceeded 100 days for all treatment groups receiving R848 gel radioimmunotherapy. All mice were able to reject tumor formation at CT26 cancer cell re-challenge after day 100. All formulations and therapeutic interventions were well tolerated.

### Conclusion

Radioimmunotherapy using external bean radiation therapy in combination with LAP, SuBen, LOIB A and LOIB B R848 releasing gel formulations provides a highly effective immune-therapeutic intervention capable of inducing durable rejection of established tumors.

### Example 6: The Dosing Schedule for R848 LOIB Gel Radioimmunotherapy does not Influence Therapeutic Efficacy

The aim of the current example is to investigate the effect of the R848 LOIB gel dosing schedule relative to initiation of radiotherapy (RT). In this example, R848 containing LOIB gel was intratumorally administered either 1H or 4H before the first RT dose, or 24H or 72H after the first RT dose.

### Methods:

A LOIB gel (LOIB:GTO:ETOH (82.5:7.5:10 (% w/w)) was prepared as described in example 2.

The efficacy study was conducted as described in example 5, with a modified R848 gel administration schedule. R848 containing gels were intratumorally administered either 1H or 4H before the first RT dose, or 24H or 72H after the first RT dose (FIG. 4A).

### Results and Discussion

The complete dosing schedule is displayed in FIG. 4A and obtained efficacy data are compiled in FIG. 4B-C. The median survival and total number of long-term survivors was increased for all groups receiving R848 LOIB gel radioimmunotherapy compared to control (NT) and RT monotherapy. No statistically significant difference was observed between the treatment schedules indicating little or no impact of whether the R848 LOIB gel was administered 1H or 4H prior to RT or 24H or 72H after the first RT dose.

### Conclusion

The combination of R848 gel immunotherapy and RT show improved efficacy compared to control and RT monotherapy independently of the tested treatment schedules.

### Example 7: Effects of R848 LOIB Gel Dosing Frequency and Volume on Therapeutic Efficacy of R848 Gel Radioimmunotherapy

The aim of the current example is to investigate the effect of the gel dosing frequency and gel volume while keeping the R848 dose constant (12 mg/kg). In this example, 25 μL or 50 μL R848 gel R848 gel immunotherapy was administered intratumorally as weekly doses of 3 mg/kg for a total of four treatments, biweekly doses of 6 mg/kg for a total of two treatments or one dose of 12 mg/kg. IL-6 cytokine levels in plasma were furthermore monitored as a measure of toxicity.

### Methods:

A LOIB gel (LOIB:GTO:ETOH 82.5:7.5:10 (% w/w)) was prepared as described in example 2.

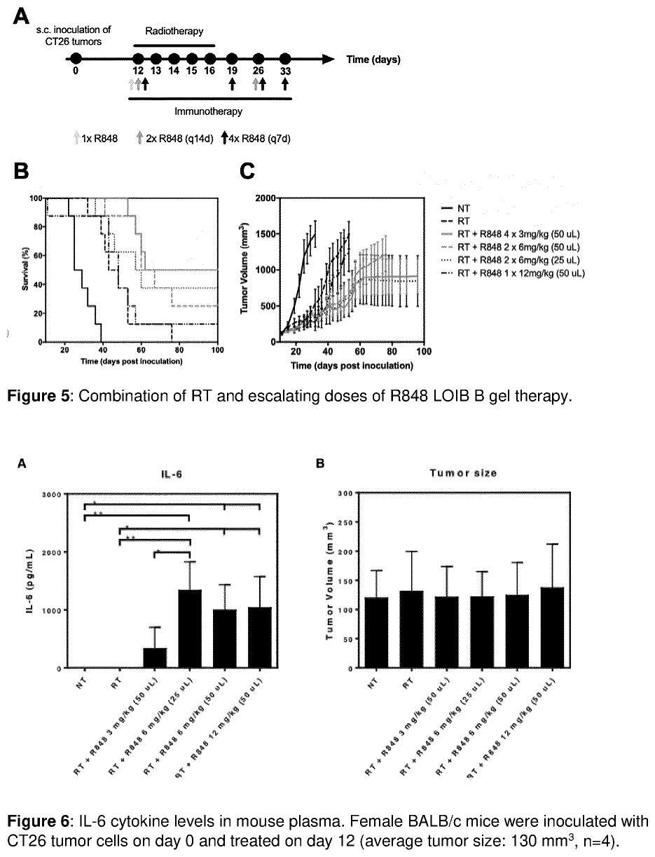

The efficacy study was conducted as described in example 5, with a modified R848 gel dosing regime. Volumes of 25 μL or 50 μL R848 gel therapy was administered intratumorally as weekly doses of 3 mg/kg for a total of four treatments, biweekly doses of 6 mg/kg for a total of two treatments or as a single dose of 12 mg/kg. (FIG. 5A).

For the mice receiving R848 gel radioimmunotherapy, RT was initiated one hour after R848 treatment. Blood samples were drawn by puncture of the sublingual vein with a 27 G needle 3 hours after administration of R848 gel therapy. For the RT monotherapy group, the blood samples were drawn 30 minutes after radiation. The blood was collected in eppendorf tubes containing ethylenediaminetetraacetic acid (EDTA) diluted in PBS to avoid blood coagulation. Blood samples were centrifuged at 5000 g for 3 minutes to separate plasma from blood cells and plasma was freezed at −80° C. The IL-6 levels were measured by enzyme-linked immunosorbent assay (ELISA) (mouse IL-6 DuoSet ELISA, R&D Systems) according to the manufacturer's protocol. Absorbance was measured at 450 and 540 nm with a FLUOstar Omega microplate reader (BMG LABTECH). PBS, Bovine serum albumin and Tween® 20 were purchased from Sigma-Aldrich. Substrate Solution and Stop Solution for ELISA were purchased from R&D Systems and EDTA was purchased from Ambion.

### Results and Discussion

The obtained therapeutic efficacy data and IL-6 data are summarized in FIG. 5-6 and table 4. The efficacy data shown in FIG. 5 and table 4 display that 4×3 mg/kg R848 gel radioimmunotherapy results in improved survival and increases the percentage of long-term survivors compared to NT and RT monotherapy. The dosing schedule 4×3 mg/kg was found to optimal with almost equal efficacy for 2×6 mg/kg (25 μL) and 2×6 mg/kg (50 μL). The 1×12 mg/kg R848 gel radioimmunotherapy resulted in shortest survival and lowest increase in long-term survivors of the R848 gel radioimmunotherapy groups. Neither the NT nor the RT group had detectable blood levels of IL-6 (FIG. 6). Of the mice receiving R848 gel radioimmunotherapy, the groups receiving 6 mg/kg or 12 mg/kg had comparable IL-6 levels. The group receiving 3 mg/kg had lower IL-6 levels than the other R848 gel radioimmunotherapy groups (FIG. 6).

TABLE 4
Overview of groups, treatments and results from the
efficacy study evaluating the combination of RT
and escalating doses of R848 LOIB B gel therapy.

| | Parameters | | | |
|---|---|---|---|---|
| | | | Median | Long-term |
| | Treatment | Treatment | survival | survivors |
| Group | RT | R848 | [days] | [%] |
| NT | — | — | 27 | 0 |
| RT | 5 × 2 Gy | — | 45.5 | 0 |
| RT + R848 4 × | 5 × 2 Gy | 4 × 3 mg/kg | 81 | 50 |
| 3 mg/kg (50 μL) | | | | |
| RT + R848 2 × | 5 × 2 Gy | 2 × 6 mg/kg | 63.5 | 25 |
| 6 mg/kg (50 μL) | | | | |
| RT + R848 2 × | 5 × 2 Gy | 2 × 5 mg/kg | >100 | 50 |
| 6 mg/kg (25 μL) | | | | |
| RT + R848 1 × | 5 × 2 Gy | 1 × 12 mg/kg | 53 | 17 |
| 12 mg/kg (50 μL) | | | | |

### Conclusion

Comparison of a several R848 LOIB gel dosing schedules during R848 gel radioimmunotherapy resulted in best outcome for 4×3 mg/kg gel group. Considerably higher IL-6 production was observed for the 2×6 mg/kg and 1×12 mg/kg dosing schedule compared to the 4×3 mg/kg dosing schedule. The dosing schedule using 3 mg/kg R848 gel radioimmunotherapy seems optimal for radioimmunotherapy of murine tumors.

### Example 8: Inclusion of Iodinated Lipids in the Gel-Formulation Provides Stable Radiographic Contrast for Optional Imaging Using Radiography Imaging Modalities

The aim of the current example is to demonstrate that iodinated lipids can be incorporated stably in the gel-formulation. The incorporation would provide optional radiography based imaging capabilities to allow for guidance of therapeutic interventions and monitoring gel position and planning of, and image guidance during, repeated administrations.

### Methods:

Gel formulations containing Lipiodol was prepared by similar mixing methods as described in example 2. Gel compositions with 15% or 25% w/w lipiodol were prepared. One formulation had the composition LOIB:Lipiodol:EtOH (75:15:10) and the second formulation had the composition SuBen:Lipiodol:EtOH (60:25:15). All ratios are weight by weight.

Female Balb/C mice, 12 weeks of age, were injected with 75 μL Lipiodol-gel formulation (LOIB or SuBen based) and micro computed tomography (CT) scans (NanoScan, Mediso, Budapes,Hungary) were performed at 10 minutes, 30 minutes, 2 hours and 2 months after the injection. For injections mice were shaved lumbar region and aseptically prepared injection. Subcutaneous injections were performed with mice under anesthesia (3-5% sevoflurane) mice using 1 ml syringe and 23 G/25 mm needle. The needle was advanced approximately 1 cm in the subcutaneous space and the formulation was slowly injected. The needle was left in place for 1 minute before being slowly retracted. CT scans were performed with mice under anesthesia (3-5% sevoflurane) and fixated on a dedicated small animal imaging bed with built heating. Houndsfield unit correct CT scans were analysed in commercially available software (Inveon, Siemens, Erlangen, Germany). In short, a volume of interest was placed around the injected formulation, taking care not to include any adjacent bony structures. The constructed volume of interest was segmented using a lower thresholds of 500 HU. The segmented volume of interest was visually inspected to include the injected formulation, which for both 15% and 25% Lipiodol was easily identified. From the segmented volume of interest radiographic mean and maximum contrast (HU) and volume was determined as a function of time.

### Results and Discussion

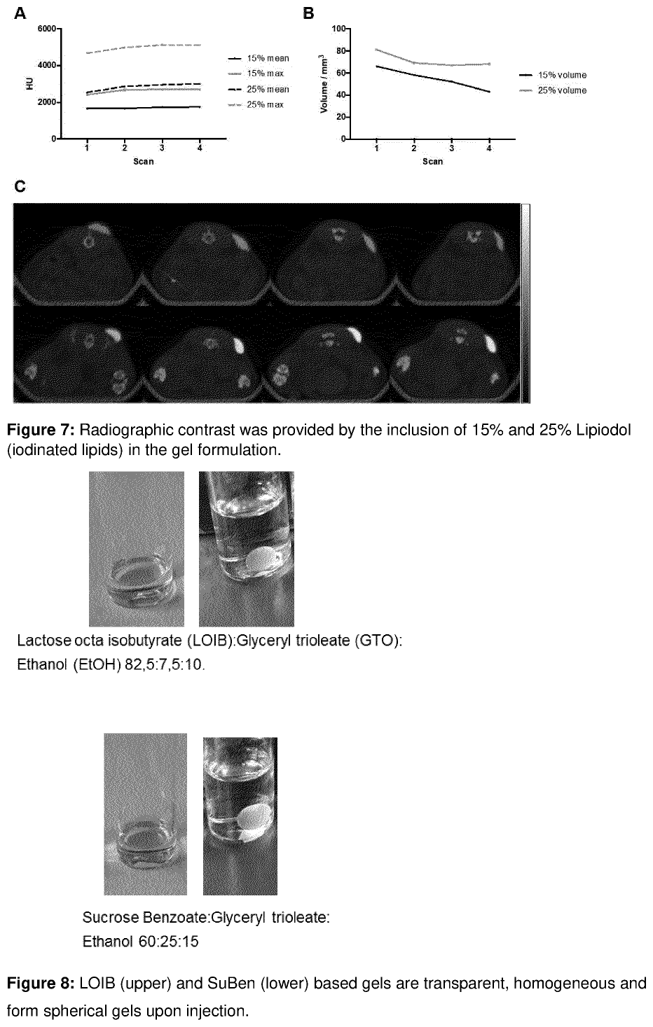

The inclusion of Lipiodol in the formulation provided high and stable radiographic contrast throughout the study period. The marker displayed increased radiographic contrast and decreasing volume which is compatible with the initial diffusion of solvent from the marker and slow degradation of the solidified gel (FIG. 7A-B). The contrast levels are sufficient for visualization on radiography-based imaging equipment (FIG. 7C). The possibility to non-invasively locate the gel can be used for optimal placement of gels at repeated injections and may serve to guide therapeutic interventions. The formulation was well tolerated and no adverse reactions were observed.

### Conclusion

Lipiodol was successfully formulated in both LOIB and SuBen based gels, and displayed constant radiographic contrast over the study period of two months for both formulations.

### Example 9. SuBen and LOIB Form Transparent Gels that Set Upon Injection into Buffer

The aim of the current example is to generate stable transparent injectable gels that solidifies upon injection.

### Methods:

LOIB:GTO:EtOH (82.5:7.5:10) weight ratios were mixed and solubilized in a water bath sonicator at 75° C. for 1 hour to form a viscous fluid. SuBen:PLA:GTO:EtOH (59.5:0.5:25:15) weight ratios were mixed and solubilized in a water bath sonicator at 75° C. for at least two hours to form a viscous fluid. Approximately 100 μL of the gel formulation were injected into 2 ml of PBS buffer using 21 G needles to form a spherical gel (lower right picture)

### Results:

LOIB and SuBen based gels were prepared and injected into PBS buffer. Representative image of gel before and after injection are presented in FIG. 8.

Homogenous and transparent gels could be produced by simple mixing of compounds followed by heating and sonication (FIG. 8). The gels could be injected into aqueous solutions and formed spherical gels.

### Conclusion:

The SuBen and LOIB gels described in this example were all transparent and injectable. The solutions formed spherical gel depots upon injection.

### Example 10. The Fraction of the Oil (Co-Solvent), GTO Determines the R848 Release Rate from LOIB Gels

The aim of the current example is to investigate the effect of GTO concentration on release kinetics of R848.

### Methods:

LOIB:GTO:ETOH gels were produced with the weight ratios 75:15:10, 80:10:10 and 82.5:7.5:10 as described in example 9, and 1.5 mg R848 per gram gel was solubilized in the gels. Briefly, 750 mg of Lactose octa isobutyrate (LOIB) was mixed with 150 mg glyceryl trioctanoate (GTO), 100 mg of ethanol (EtOH) and 1.5 mg of R848. 800 mg of Lactose octa isobutyrate (LOIB) was mixed with 100 mg glyceryl trioctanoate (GTO), 100 mg of ethanol (EtOH) and 1.5 mg of R848. 825 mg of Lactose octa isobutyrate (LOIB) was mixed with 75 mg glyceryl trioctanoate (GTO), 100 mg of ethanol (EtOH) and 1.5 mg of R848.

Following, 100 μL of each gel were injected in duplicates into 2 ml of PBS. Cumulative release of R848 as a function of time was measured μl of by fluorescence (ex. 330 nm, em. 355 nm).

### Results and Discussion

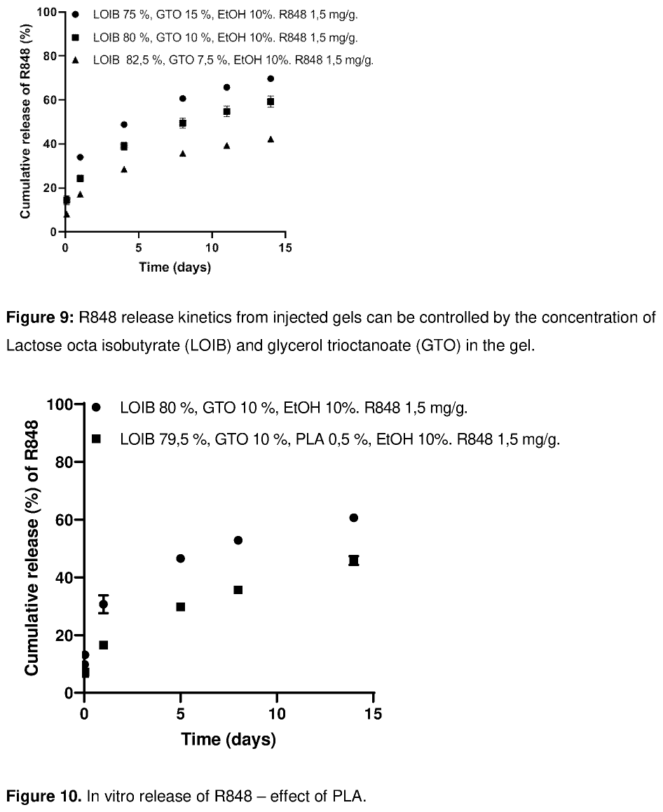

LOIB:GTO gels were prepared and 100 μL were injected into PBS buffer for study of the in vitro release of R848. The in vitro release results are compiled in FIG. 9.

The LOIB-based gel formulations have ideal viscosity when used in weight % concentrations between 75-82.5%. To investigate release kinetics of R848 from three formulations containing 75%, 80% and 82.5% (% w/w) of LOIB, 1.5 mg R848 per gram gel was solubilized in the gels and 100 μl of each gel was injected in duplicates into 2 ml of PBS. Samples were taken at fixed time points as described and the amount of released R848 was measured. We could observe that both initial and total release was dependent on the content of GTO in the gels. The difference in total cumulative release was almost 30% between LOIB 75% and LOIB 82.5%(% w/w)

### Conclusion

The data show that the maximal cumulative release of R848 obtained after 14 days differed from around 70% for the 75% (% w/w) LOIB formulation (15% GTO) to 42% for the 82.5% (% w/w) LOIB formulation (7.5% GTO). This means that depending on the wanted release profile we can tune the kinetics by changing the GTO content in the gels.

### Example 11. Inclusion of PLA in LOIB Gels Reduces Burst Release of R848

The aim of the current example is to investigate the effect of PLA inclusion in LOIB-based gels on release kinetics of R848.

### Methods:

LOIB:GTO:EtOH (80:10:10 (% w/w) containing 1.5 mg/mL R848 was prepared as described in example 9. Briefly 800 mg of Lactose octa isobutyrate (LOIB) was mixed with 100 mg glyceryl trioctanoate (GTO), 100 mg of ethanol (EtOH) and 1.5 mg of R848.

LOIB:GTO:EtOH:PLA (79.5:10:10:0.5 (% w/w) gel containing 1.5 mg/mL R848 was prepared as described in example 9. Briefly, 795 mg of Lactose octa isobutyrate (LOIB) was mixed with 100 mg glyceryl trioctanoate (GTO), 5 mg of poly lactic acid (PLA) Mw 8-18 kDa, 100 mg of ethanol (EtOH) and 1.5 mg of R848.

Following, 100 μL of each gel were injected in duplicates into 2 ml of PBS. Cumulative release of R848 as a function of time was measured by fluorescence (ex 330 nm, em 355 nm).

### Results and Discussion

LOIB:GTO:EtOH gels with and without PLA were prepared and 100 μL was injected into PBS buffer for study of the in vitro release of R848. The in vitro release results are compiled in FIG. 10.

Another way of controlling the drug release can be the inclusion of polymers in the gels such as PLA. Therefore 0.5 (% w/w) of PLA was complemented to a 79.5 (% w/w) LOIB formulation and R848 release was compared with an 80 (% w/w) LOIB formulation. We observed that 0.5 (% w/w) of PLA could reduce both the early burst release and overall release after 14 days showing that the addition of PLA to the gels regulates the release kinetics.

### Conclusion

The data show that addition of 0.5 (% w/w) PLA to LOIB gels reduced the burst release of R848 with around 50% and the total cumulative release was reduced from 60% to less than 50%.

### Example 12. R848 Release Profile from LOIB Gels is Independent of Initial R848 Gel Concentration

The aim of the current example is to investigate the effect in release of different concentrations of R848 incorporated into LOIB-based gels.

### Methods:

LOIB:GTO:EtOH (82.5:7.5:10 (% w/w)) gel containing either 1.5, 5.0 or 10 mg/g R848 was prepared as described in example 9. Briefly, 825 mg of Lactose octa isobutyrate (LOIB) was mixed with 75 mg glyceryl trioctanoate (GTO), 100 mg of ethanol (EtOH) and 1.5, 5 or 10 mg of R848.

Following, 100 μL of each gel was injected in duplicates into 2 ml of PBS. Cumulative release of R848 as a function of time was measured by fluorescence (ex 330 nm, em 355 nm).

### Results and Discussion

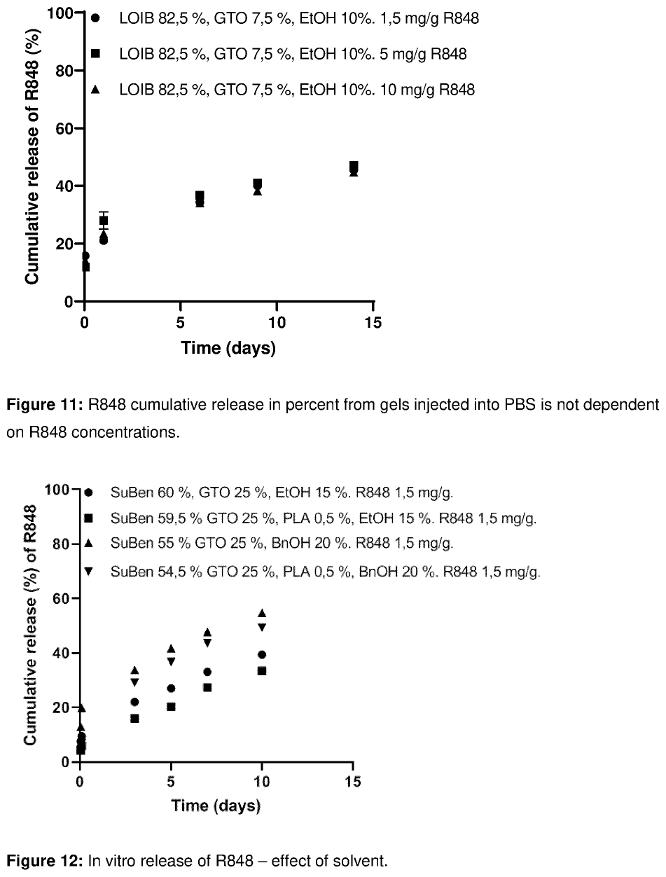

LOIB:GTO:EtOH gels with increasing R848 concentration were prepared and 100 μL was injected into PBS buffer for study of the in vitro release of R848. The in vitro release results are compiled in FIG. 11.

LOIB 82.5 (% w/w) was used to solubilize 1.5, 5 and 10 mg R848/g gel. 100 μl of each gel was injected in duplicates into 2 ml of PBS. Samples were taken at fixed time points and the amount of released R848 was measured using fluorescence spectrometry (all percentages are (% w/w)).

The relative cumulative release (given in percent) from Lactose octa isobutyrate (LOIB) gels is observed to be independent of the initial R848 gel concentration (FIG. 11). The absolute release of R848 from the gel upon injection in PBS buffer is therefore proportional to the initial R848 concentration in the gel.

### Conclusion:

For LOIB:GTO:EtOH (82.5:7.5:10 (% w/w)) gels, the relative cumulative release of R848 is independent of the initial R848 concentration in the gel, whereas the absolute release of R848 is proportional to the R848 gel concentration.

### Example 13: The SuBen Gel Concentration and the Combination with Benzyl Alcohol and PLA can Control the In Vitro Release of R848 from Gels

The aim of the current example is to investigate release of R848 in SuBen-based gels produced with EtOH or BNOH as solvent and with the addition of 0.5 (% w/w) PLA.

### Methods:

SuBen:GTO:EtOH (60:25:15 (% w/w)) Gel was prepared as described in example 9. Briefly, 600 mg of Sucrose benzoate (SuBen) was mixed with 250 mg glyceryl trioctanoate (GTO) and 150 mg of ethanol (EtOH) and 1.5 mg of R848.

SuBen:GTO:PLA:EtOH (59.5:25:0.5:15 (% w/w)) Gel was prepared as described in example 9. Briefly, 595 mg of Sucrose benzoate (SuBen) was mixed with 250 mg glyceryl trioctanoate (GTO) 5 mg of poly lactic acid (PLA) Mw 8-18 kDa, 150 mg of ethanol (EtOH) and 1.5 mg of R848.

SuBen:GTO:BnOH (55:25:20 (% w/w)) Gel was prepared as described in example 9. Briefly, 550 mg of Sucrose benzoate (SuBen) was mixed with 250 mg glyceryl trioctanoate (GTO), 200 mg of Benzylalcohol and 1.5 mg of R848.

SuBen:GTO:PLA:BnOH (54.5:25:0.5:20 (% w/w)) Gel was prepared as described in example 9. 545 mg of Sucrose benzoate (SuBen) was mixed with 250 mg glyceryl trioctanoate (GTO), 200 mg of Benzylalcohol, 5 mg of poly lactic acid (PLA) Mw 8-18 kDa and 1.5 mg of R848.

Following, 100 μL of each gel were injected in duplicates into 2 ml of PBS. Cumulative release of R848 as a function of time was measured by fluorescence (ex 330 nm, em 355 nm).

### Results and Discussion

Suben:GTO gels were prepared with EtOH or BnOH (Benzyl alcohol, BA) and 1.5 mg R848/1 g gel was solubilized. In vitro release was conducted in PBS buffer and the results are compiled in FIG. 12.

The SuBen-based gel formulations have ideal viscosity when used in weight % concentrations between 50-60 weight %. The gels formulated with benzylalcohol could only contain 55 (% w/w) of SuBen otherwise the formulation was too viscous. To investigate release kinetics of R848 from four formulations containing 60 (% w/w), 59.5 (% w/w), 55 (% w/w) or 54.5 (% w/w) of SuBen and solubilized with ethanol or benzylalcohol and with or without poly lactic acid (PLA). The results showed that benzyl alcohol increased the release of R848 compared to ethanol and the incorporation of PLA could reduce R848 release meaning that both the choice of solvent and the addition of PLA can be used to control release.

### Conclusion:

The Sucrose benzoate concentration and the combination with Benzyl alcohol and PLA can control the release of R848 from injected gels.

### Example 14. In Vitro Release of RepSox and Galunisertib from LOIB Based Gels

The aim of the current example is to investigate release of the TGFβ inhibitors RepSox and Galunisertib in LOIB-based gels.

### Methods:

LOIB:GTO:EtOH (82.5:7.5:10 (% w/w)) gel containing either 8 mg RepSox or Galunisertib was prepared as described in example 9. Briefly, 825 mg of Lactose octa isobutyrate (LOIB) was mixed with 75 mg glyceryl trioctanoate (GTO), 100 mg of ethanol (EtOH) and 8 mg of RepSox or Galunisertib.

Following, 100 μL of each gel were injected in duplicates into 2 ml of PBS. Cumulative release of RepSox and Galunisertib as a function of time was measured by absorbance at 330 nm and 280 nm.

### Results and Discussion

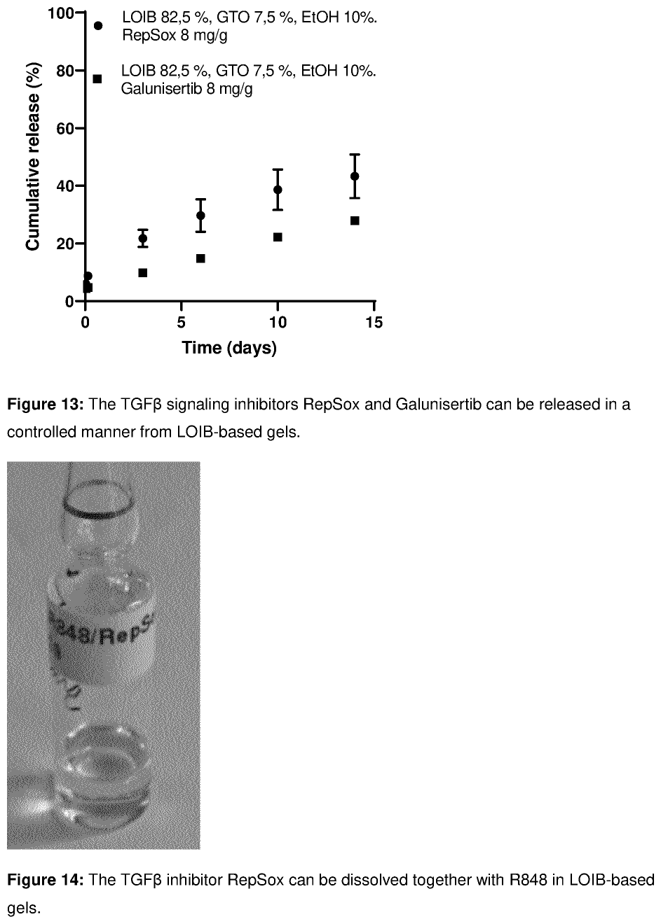

LOIB:GTO gels containing either 8 mg RepSox/1 g gel or 8 mg Galunisertib/1 g gel were prepared successfully and in vitro release into PBS buffer was studied. Release results are compiled in FIG. 13.

RepSox and Galunisertib are TGFβ signaling inhibitors that are interesting to use in combination with TLR 7 agonists such as R848 as immune therapy for cancer treatment. These drugs can be solubilized in Lactose octa isobutyrate (LOIB)-based gel formulations at 8 mg/gram or more. Therefore 8 mg/gram of RepSox or Galunisertib was solubilized in 82.5 (% w/w) LOIB gels. Both drugs released from the gels in a linear fashion and RepSox cumulative release was around 40% after 14 days. Galunisertib released slower with a release of 27% after 14 days.

### Conclusion:

The data show that the TGFβ signaling inhibitors RepSox and Galunisertib can be incorporated in LOIB-based gels and be released up to 40% after 14 days.

### Example 15: Co-Solubilization of R848 and RepSox in LOIB Gels

The aim of the current example is to test if the TGFβ inhibitor RepSox could be co-dissolved with R848 in LOIB-based gels.

### Methods:

A LOIB:GTO:EtOH (82.5:7.5:10 (% w/w)). gel was produced as described in example 9. Following, 2.4 mg R848 and 8.0 mg RepSox was weighed into a vial, and 1 g of gel was added. The mixture was solubilized using a magnetic stirring for 2 hours at 40-50° C. under continuous visual inspection.

### Results and Discussion

The LOIB:GTO:EtOH gel was successfully prepared and R848 and RepSox was co-dissolved in the gel as displayed in FIG. 14.

RepSox is a TGFβ signaling inhibitor that is interesting to use in combination with TLR 7 agonists such as R848 for immune therapy for cancer treatment. Therefore, we tested if the two drugs could be dissolved in the same gel at a high concentration to obtain a combination gel. 2.4 mg of R848 and 8 mg/g could readily be dissolved together and a weakly yellowish solution was obtained.

### Conclusion:

The data show that it is possible to co-dissolve RepSox and R848 at high concentrations in the same gel.

### Example 16: Combined Radiation Therapy and Intratumoral Immunotherapy (Radioimmunotherapy) Using Multi-Drug Releasing Intratumoral Gels Demonstrate the Therapeutic Potential of a Multitargeted Approach

The aim of the current example is to demonstrate that the LOIB and SuBen based gel formulations can provide improved therapeutic efficacy by providing controlled release of multiple immunotherapeutic drug in tumors undergoing radioimmunotherapy.

### Methods:

The gels used in this study were LOIB:GTO:EtOH (82.5:7.5:10 (% w/w)) for RepSox and Galunisertib in combination with R848 and SuBen:GTO:PLA:EtOH (59.5:25:0.5:15 (% w/w)) for SD-208 in combination with R848. The gels were prepared as previously described and thereafter added on top of freeze-dried TGFβ inhibitors together with R848. The drugs were dissolved at 40° C. using a magnetic stirrer until a clear gel was obtained. The TGFβ inhibitors had a final concentration of 8 mg/g and the co-dissolved R848 was 1.2 mg/g.

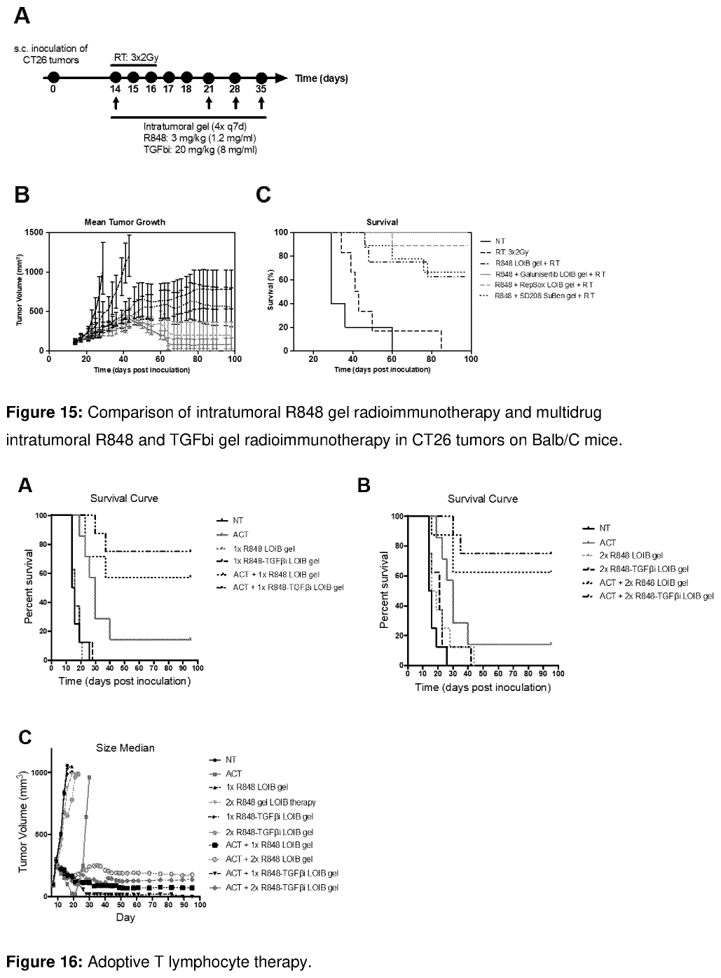

6-7-week-old female BALB/c mice were inoculated with 3×10⁵ CT26 tumor cells in 100 μl of RPMI on the right flank by subcutaneous injection on day 0. Tumors were allowed to grow until their average size exceeded 100 mm³ (day 14) before the mice were divided into treatment groups based on a size-dependent randomization (n=8/group). Mice in the R848 monotherapy group were treated with R848 LOIB gel administered as weekly doses of 3 mg/kg for a total of four treatments. Mice in R848 and transforming growth factor beta signalling inhibitors (TGFbi) groups were treated with either R848+TGFbi LOIB gel (TGFbi; Galunisertib or RepSox) or R848+TGFbi SuBen gel (SD-208) administered as weekly doses of 3 mg/kg R848 and 20 mg/kg TGFbi for a total of four treatments (please refer to FIG. 15A for treatment overview). The gel was administered by intratumoral injections of 50 μL and mice were anesthetized by inhalation anesthesia (˜4.5% sevoflurane). Radiation was delivered in three fractions of 2 Gy. Prior to radiation, the mice were anesthetized (˜4.5% sevoflurane) and fixated with a shielding device only exposing the right tumor bearing leg. Radiation was delivered with a dose rate of 1 Gy/minute (12.5 mAs/320 kV) using a dedicated small animal irradiation device (X-RAD 320, Precision X-Ray, Inc.). Gel therapies were administered one hour prior to RT. Both tumor and bodyweight measurements were collected 3 times per week. Mice were terminated from the study once their tumor reached a tumor volume 1000. Additional, study endpoints included weight loss >15% and presence of extensive ulcerations on the tumors. The tumors were measured by a digital caliper and the tumor volumes were calculated using the equation (length×width2)/2. Mice that survived more than 100 days post inoculation were defined as long-term survivors. Mice designated as long-term survivors were rechallenged after day 100 with CT26 cancer cells on the left flank (3×10⁵ CT26 tumor cells in 100 μl of RPMI by subcutaneous injection) along with naïve mice to determine if sufficient immunologic memory has been raised to reject tumor establishment. None of the included mice displayed adverse reactions and the combined radioimmunotherapy was well tolerated across groups.

### Results and Discussion

The combined radioimmunotherapy was highly effective at controlling tumor growth (FIG. 15B) and induced complete rejection (survival) of tumor in the majority of mice across all group receiving external beam radiation therapy and immunotherapeutic gels (FIG. 15C). The groups receiving the multitargeted immunotherapy LOIB gels containing both R848 and TGFbi inhibitors Galunisertib or RepSox displayed complete rejection of tumors in 8/8 (LOIB gel R848+Galunisertib) and 7/8 mice (LOIB gel R848+RepSox). The groups receiving radioimmunotherapy using LOIB gel R848 and the combined SuBen gel R848+SD-208 induced complete rejection of 5/8 mice in each group. All tumor free mice were able to reject tumor rechallenge indicating that durable immunological memory has been established. The presented results indicate that gels providing release of multiple immunotherapeutics may provide improved therapeutic efficacy with compromising tolerability. These observations indicate that the controlled release intratumoral gel formulation may provide the highly attractive multitargeted immunotherapeutic approach to overcome the hostile immunosuppressive tumor microenvironment.

### Conclusion

The presented example demonstrates that the providing controlled intratumoral release of multiple immunotherapeutic may improve therapeutic efficacy with compromising tolerability.

### Example 17. The Gel Formulations have Viscosity Between 290 and 570 mPa s at 37° C.

The aim of the current example is to investigate the viscosity of LOIB and SuBen-based gels.

### Methods

LOIB:GTO:EtOH (80:10:10 (% w/w)), LOIB:GTO:EtOH (82.5:7.5:10 (% w/w)) and SuBen:GTO:PLA:EtOH (59.5:25:0.5:15) gels were prepared as in example 2. Viscosity measurements were performed in glass vials with a metal ball (2 mm in diameter) and at least 300 μL of gel material. For each measurement the balls were spun at 1000 revolutions per minute for 1 minute at 25° C. or 37° C. (EMS-1000 Electro Magnetically Spinning Viscometer, Kyoto Electronics).

### Results and Discussion

The LOIB and SuBen based gels were prepared successfully and the viscosity was determined. The results are compiled in table 5.

TABLE 5
Example of viscosity measurements at 25° C. and 37°
C. Unit: mPa s or cP, (all percentages are (% w/w)).

| Formulation | Viscosity at 25° C. [Pa S] | Viscosity at 37° C. [Pa S] |
|---|---|---|
| LOIB 80% | — | 475 |
| LOIB 82.5% | 1160 | 570 |
| SuBen 59.5% | — | 288 |

The viscosity measurements performed on the different gels at 37° C. showed that the addition of 2.5 (% w/w) GTO to the LOIB gels resulted in a reduced viscosity of 100 mPa s. The SuBen-based gel was the most fluid at 37° C. A viscosity of the gels between 200-1500 mPa s is suitable for injection into animal or human tissues. The LOIB:GTO:EtOH (82.5:7.5:10 (% w/w)) formulation was also tested at 25° C. and was 1160 mPa s, around a doubling from the results obtained at 37° C. but still within the range suitable for injection.

### Conclusion

The LOIB and SuBen based gels tested in this experiment showed viscosity numbers between 288 and 570 mPa s at 37° C. and that is compatible with controlled injections into tissues.

### Example 18: R848 LOIB Gel and R848 TGFßi LOIB Gel Potentiates the Effect of Adoptive T Lymphocyte Therapy

The aim of the current example is to demonstrate how intratumoral R848 LOIB gel and R848+TGFßi (RepSox) LOIB gel potentiates the therapeutic efficacy of adoptive T lymphocyte therapy. Furthermore, the influence on repeated dosing of immune stimulating gels was investigated.

### Methods:

The gels used in this study were LOIB:GTO:EtOH (82.5:7.5:10 (% w/w)) for R848 as single drug or in combination with RepSox (TGFβi). The gels were prepared as previously described and thereafter added on top of freeze-dried RepSox inhibitor together with R848 for the multidrug gel. The drugs were dissolved at 40° C. using a magnetic stirrer until a clear gel was obtained. The TGFβ inhibitor (RepSox) had a final concentration of 8 mg/g and the co-dissolved R848 was 1.2 mg/g.

6-7-week-old female C57BL/6 mice were inoculated with 3×10⁵ EG7.OVA cancer cells in 100 μl of RPMI on the right flank by subcutaneous injection on day 0. Tumors were allowed to grow until their average size exceeded 100 mm³ (day 7) before the mice were divided into treatment groups based on a size-dependent randomization (n=7-8/group). The following groups were included in the therapeutic study: No treatment controls, OVA specific OT.1 CD8+T lymphocytes, R848 LOIB gel single dose, R848 LOIB gel two doses placed 7 days apart, R848+TGßi LOIB gel single dose, R848+TGFßi LOIB gel two doses placed 7 days apart or the combination of OVA specific OT.1 T lymphocytes and R848 LOIB gel single dose, OVA specific OT.1 T lymphocytes and R848 LOIB gel two doses placed 7 days apart, OVA specific OT.1 T lymphocytes and R848+TGFßi LOIB gel single dose and OVA specific OT.1 T lymphocytes and R848+TGßi LOIB gel two doses placed 7 days apart. For all treatments with OVA specific OT.1 T lymphocytes a single intravenous dose of 10⁶ T cells was used. All LOIB gels were administered intratumorally at a volume of 50 μL with mice under general anaesthesia (sevoflurance 3-5%). Doses of 3 mg/kg R848 and 20 mg/kg TGFßi were administered. Tumor and bodyweight measurements were performed 3 times per week. Mice were terminated from the study once their tumor reached a tumor volume 1000. Additional, study endpoints included weight loss >15% and presence of extensive ulcerations on the tumors. The tumors were measured by a digital caliper and the tumor volumes were calculated using the equation (length×width2)/2. Mice that survived more than 100 days post inoculation were defined as long-term survivors.

### Results and Discussion

The inclusion of the R848 LOIB gel and combined R848+TGFßi LOIB gel potentiated the therapeutic efficacy of adoptive OVA specific OT.1 T lymphocyte therapy. In the group only receiving adoptive T lymphocytes only 1/8 mice displayed complete rejection of its tumor and none of the mice treated with only R848 or R848+TGFßi LOIB gels were able to reject their tumors. On the contrary, the combination of OVA specific OT.1 T lymphocytes and R848 or R848+TGßi LOIB gels induced complete rejection of tumors in the majority of mice. For the OVA specific OT.1 T lymphocytes and R848 LOIB gels 4/7 (single dose LOIB gel) and 5/8 (two doses of LOIB gel) mice displayed complete rejection of their tumors (FIG. 16A). The combination of OVA specific OT.1 T lymphocytes and R848+TGFßi LOIB gels induced complete rejection of tumors in 6/8 mice in both single dose and two doses of LOIB gel (FIG. 16B). On the tumor growth curves (FIG. 16C) of median size over time the influence of the R848LOIB and R848+TGßi LOIB gels can be readily appreciated. As seen from the curves the OVA specific OT.1 T lymphocytes induces a rapid reduction of tumor volume in the cell only treated group, however, this response is only short lived and tumors regrow. On the contrary, the addition of R848 LOIB and R848+TGFßi LOIB gels provides a therapeutic benefit that inhibits the regrowth of tumors which directly indicates that the activity of the transferred OVA specific OT.1 T lymphocytes is optimized. Neither the R848 LOIB or R848+TGßi LOIB gel displayed any therapeutic efficacy as single therapy which further indicates that the observed effect must be associated with a potent improvement of OVA specific OT.1 T lymphocyte anti-cancer efficacy. The comparable effect of only a single administration and repeated dosing of R848 LOIB or R848+TGFßi LOIB gel demonstrate the controlled drug delivery system provides a long lived activity sufficient to support the activity of the OVA specific OT.1 T lymphocyte.

### Conclusion

The presented example demonstrates that the LOIB gel drug delivery system can deliver immune stimulating drugs directly in tumors to significantly potentiate the effect of adoptively transferred cell therapies. The observed effect is highly encouraging towards further advancing this therapeutic combination.

### Example 19: API Gel Preparation Procedure

The aim of the example is to describe the procedure of preparation of gels and solubilization of active pharmaceutical compounds (APIs) in these.

Gels are prepared by mixing of carbohydrate esters e.g. SuBen, LacBen, LOIB etc. with solvents and co-solvents e.g. EtOH, GTO, DMSO, PC etc. Additional gel components are listed in example 20 and APIs in example 21. Compositions are given in weight percent (or weight ratio) and each compound is weighed into one vial. The mixture is placed in an ultrasonication bath at 70-80° C. for 1-2 hours and occasionally vortexed to generate homogenous solutions that is subsequently stored at 4° C. until further use. Afterwards, all active pharmaceutical ingredients (APIs) are incorporated into the gels proportionally by adding gel on top of freeze-dried APIs or combinations of freeze-dried APIs. The gel is subjected to magnetic stirring at 40-50° C. until the API or combination of APIs is completely dissolved. The gel-API mixture is transferred and stored in sealed vials at room temperature or at 4° C. Certain gel-API mixtures may be stored at −20° C.

### Example 20: Mixable Gel Constituents

The aim of the current example is to describe weight-percent (or weight ratio) ranges for gel constituents within which completely dissolved gels are formed. Preferred gel compositions are also stated.

### Methods

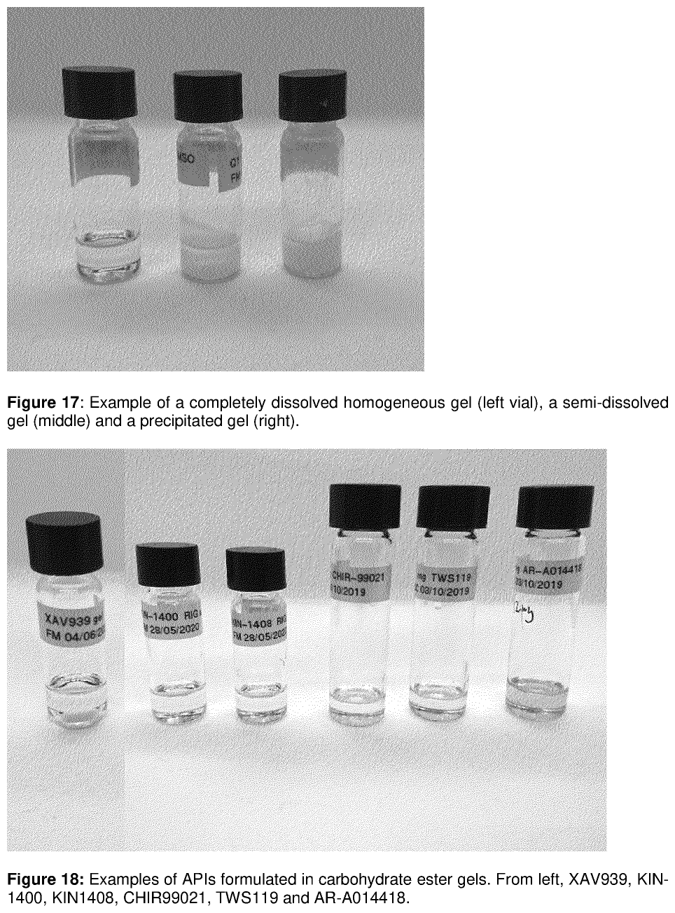

Gels were prepared as described in example 19. After settling to room temperature, the gels were visually inspected to verify if the gel solutions were completely dissolved as shown in the leftmost vial in FIG. 17.

### Results and Discussion

FIG. 17 shows a completely dissolved gel (left vial), a semi-dissolved gel (middle) and precipitated gel (right).

Table 6 and 7 below show weight ratios of formulations capable of forming completely dissolved gels. The preferred compositions have the most adequate viscosity for injection through fine needles using a syringe.

TABLE 6
Gel compositions based on SuBen (weight ratio or weight %).

| | Gel with EtOH | | Gel with PC | | Gel with BnOH | |
|---|---|---|---|---|---|---|
| Chemical | Range | Preferred | Range | Preferred | Range | Preferred |
| SuBen | 40-70 | 60 | 40-70 | 52.5 | 40-70 | 55 |
| GTO | 15-30 | 25 | 15-30 | 22.5 | 15-30 | 25 |
| EtOH | 5-30 | 15 | | | | |
| PC | | | 5-30 | 20 | | |
| BnOH | | | | | 5-30 | 20 |
| PLA (10-18 kDa) | 0.25-2 | 0-0.5 | 0.25-2 | 0-0.5 | 0.25-2 | 0-0.5 |

SuBen-based formulations can additionally be made using co-solvents Ethyl-myristate, Ethyl-palmitate or Ethyl-oleate alone or in combination with GTO.

Other tested SuBen formulations yielding homogeneous solutions are (weight ratio or weight %):

SuBen:Ethyl-myristate:EtOH (60:25:15), SuBen:Ethyl-myristate:GTO:EtOH (60:12.5:12.5:15), SuBen:Ethyl-myristate:BnOH (60:25:15), SuBen:Ethyl-palmitate:Ethyl-myristate:EtOH (62.5:11.25:11.25:15) SuBen:Ethyl-palmitate:EtOH (60:25:15), SuBen:Ethyl-palmitate:GTO:EtOH (60:12.5:12.5:15), SuBen:Ethyl-palmitate:BnOH (60:25:15), SuBen:Ethyl-oleate:EtOH (60:25:15), SuBen:Ethyl-oleate:GTO:EtOH (60:12.5:12.5:15), SuBen:Ethyl-oleate:BnOH (60:25:15).

SuBen may be replaced by RaBen or LacBen in certain compositions. The following composition have been successfully tested (w/w %):LacBen:GTO:EtOH (60:25:15), LacBen:GTO:BnOH (55:25:20), LacBen:GTO:PC (55:25:20) RaBen:GTO:EtOH (60:25:15), RaBen:GTO:BnOH (55:25:20), RaBen:GTO:PC (55:25:20).

TABLE 7
Gel composition based on LOIB (weight ratio or weight %).

| | Gel with EtOH | | Gel with PC | | Gel with BnOH | | Gel with DMSO | | Gel with Acetone | |
|---|---|---|---|---|---|---|---|---|---|---|
| Chemical | Range | Preferred | Range | Preferred | Range | Preferred | Range | Preferred | Range | Preferred |
| LOIB | 70-85 | 82.5 | 70-85 | 80 | 70-85 | 80 | 70-85 | 80 | 70-85 | 82.5 |
| GTO | 5-15 | 7.5 | 5-15 | 7.5 | 5-15 | 7.5 | 5-15 | 7.5 | 5-15 | 7.5 |
| EtOH | 5-15 | 10 | | | | | | | | |
| PC | | | 5-15 | 12.5 | | | | | | |
| BnOH | | | | | 5-15 | 12.5 | | | | |
| DMSO | | | | | | | 5-15 | 12.5 | | |
| Acetone | | | | | | | | | 5-15 | 10 |

LOIB-based formulations can alternatively be made using co-solvents: Ethyl-myristate, Ethyl-palmitate or Ethyl-oleate alone or in combination with GTO. Tested examples are (% w/w):

LOIB:Ethyl-myristate:EtOH (82.5:7.5:10, LOIB:Ethyl-myristate:GTO:EtOH (75:7.5:7.5:10)

LOIB:Ethyl-palmitate:EtOH (82.5:7.5:10), LOIB:Ethyl-palmitate:GTO:EtOH (75:7.5:7.5:10)

LOIB:Ethyl-oleate:EtOH (82.5:7.5:10), LOIB:Ethyl-oleate:GTO:EtOH (75:7.5:7.5:10).

Abbreviations: EtOH=ethanol, BnOH=benzylalcohol, PC=propylene carbonate, DMSO=dimethyl sulfoxide, GTO=glycerol trioctanoate, SuBen=sucrose octabenzoate, LOIB=lactose octa-isobutyrate, LacBen=lactose octabenzoate, RaBen=raffinose octabenzoate, PLA=poly lactic acid,

Homogeneous gels based on SuBen, LOIB, LacBen, RaBen could be produced using different solvents and co-solvents. Simple mixing of the compounds followed by heating and sonication was sufficient for producing transparent gel formulations.

### Conclusion

Homogeneous and transparent gel formulation could be formed from disaccharides carbohydrate esters such as SuBen, LOIB and LacBen or the trisaccharide carbohydrate ester RaBen together with solvents, and co-solvents.

### Example 21: Investigating Solubility of APIs in Carbohydrate Ester Gels

The aim of the current example is to show solubility of APIs in carbohydrate ester gels.

### Methods

The gels and the solubilisation of APIs were performed as described in Example 19.

### Results and Discussion

Drugs from several classes of compounds including TLR agonists, RIG-I agonists, TGFβ inhibitors, GSK3β inhibitors, Wnt-β-catenin (tankyrase) inhibitors and antibiotics such as Erythromycin have been tested and can be solubilised in gels. Examples of tested APIs are shown in table 8 and the investigated solubility is indicated. FIG. 18 shows examples of dissolved compounds in carbohydrate gels. It should be noted that the maximal solubility may be higher than the tested solubility given in table 8.

TABLE 8
Tested solubility of APIs in gel formulations. Gel
compositions are given in weight percentage/weight
ratio and API solubility in mg API per gram of gel.

| | | Tested |
|---|---|---|
| | | solubility in |
| | Gel formulations, weight | gels (mg/g |
| APIs | percentage/weight ratio. | gel) |
| Resiquimod (R848) | SuBen:GTO:EtOH (60:25:15) | 4.8 |
| (TLR agonist) | SuBen:GTO:EtOH:PLA | |
| | (60:25:15:0.5) | |
| | SuBen:Ethyl-palmitate:EtOH | |
| | (60:25:15) | |
| | SuBen:Ethyl-myristate:EtOH | |
| | (60:25:15) | |
| | SuBen:Ethyl-oleate:EtOH | |
| | (60:25:15) | |
| | SuBen:Lipiodol:EtOH (60:25:15) | |
| | SuBen:GTO:PC (60:25:15) | |
| | SuBen:GTO:BnOH (60:25:15) | |
| | SuBen:GTO:BnOH:PLA | |
| | (60:25:15:0.5) | |
| | SuBen:GTO:BnOH:PLGA | |
| | (60:25:15:0.5) | |
| | LOIB:GTO:EtOH (70:20:10), | |
| | (80:10:10), (82.5:7.5:10), | |
| | (85:5:10) | |
| | LOIB:GTO:EtOH:PLGA | |
| | (70:20:10:0.5), (80:10:10:0.5) | |
| | LOIB:GTO:EtOH:PLA (80:10:10:1 | |
| | LOIB:Lipiodol:EtOH (80:15:5) | |
| | LOIB:SuBen:GTO:EtOH | |
| | (35:35:20:10) | |
| | LOIB:GTO:EtOH:DSPE-PEG2000 | |
| | (70:20:10:0.5), (70:20:10:1) | |
| | LOIB:GTO:EtOH:DOTAP | |
| | (70:20:10:0.5), (70:20:10:1) | |
| | LOIB:GTO:EtOH:DOTAP:PLA | |
| | (75:15:10:1) | |
| | Methoxy-LOIB:GTO:EtOH | |
| | (80:10:10) | |
| | LacBen:GTO:EtOH (60:25:15) | |
| | ROIB:GTO:EtOH (80:10:10) | |
| | TOIB:GTO:EtOH (80:10:10) | |
| | SOIB:GTO:EtOH (80:10:10) | |
| | CLA-1:GTO:EtOH (82.5:7.5:10) | |
| Imiquimod (TLR | SuBen:GTO:BnOH (60:25:15) | 9.6 |
| agonist) | SuBen:GTO:EtOH:PLA | |
| | (60:25:15:0.5) | |
| Gardiquimod (TLR | LOIB:GTO:EtOH (82.5:7.5:10) | 5 |
| agonist) | SuBen:GTO:BnOH (60:25:15) | |
| RepSox (TGFβ | LOIB:GTO:EtOH (82.5:7.5:10) | 8 |
| inhibitor) | SuBen:GTO:EtOH:PLA | |
| | (60:25:15:0.5) | |
| Galunisertib (TGFβ | LOIB:GTO:EtOH (82.5:7.5:10) | 8 |
| inhibitor) | SuBen:GTO:EtOH:PLA | |
| | (60:25:15:0.5) | |
| SD-208 (TGFβ | SuBen:GTO:EtOH:PLA | 8 |
| inhibitor) | (60:25:15:0.5) | |
| | LOIB:SuBen:GTO:EtOH | |
| | (35:35:20:10) | |
| KIN-1400 (RIG-I | SuBen:GTO:EtOH (60:25:15) | 2 |
| agonist) | | |
| KIN-1408 (RIG-I | SuBen:GTO:EtOH (60:25:15) | 2 |
| agonist) | | |
| CHIR-99021 (GSK3β | SuBen:GTO:EtOH (60:25:15) | 1 |
| inhibitor) | | |
| TWS119 (GSK3β | SuBen:GTO:EtOH (60:25:15) | 1 |
| inhibitor) | | |
| AR-A014418 (GSK3β | SuBen:GTO:EtOH (60:25:15) | 1 |
| inhibitor) | | |
| TDZD-8 (GSK3β | LOIB:GTO:EtOH (82.5:7.5:10) | 1 |
| inhibitor) | | |
| XAV939 (Wnt-β- | SuBen:GTO:EtOH (60:25:15) | 1 |
| catenin inhibitor) | | |
| Doxorubicin | SuBen:GTO:EtOH (60:25:15) | 1 |
| (Topoisomerase II | LOIB:GTO:EtOH (82.5:7.5:10) | |
| inhibitor) | | |
| Erythromycin | SuBen:GTO:EtOH (60:25:15) | 150 |
| (antibiotic) | LOIB:GTO:EtOH (82.5:7.5:10) | |

### Conclusion

Carbohydrate ester based gels containing solvents and co-solvents can dissolve a large variety of active compounds at relevant concentrations making the gel system suitable as injectable drug depots.

### Example 22: Gel Formulations Incorporating CT Contrast Agents CLA-1 and CLA-8

The aim of the current example is to investigate the solubility of CLA-1 (3-iodobenzyl LOIB, 3-iodobenzyl hepta-O-isobutyryl-α,β-lactoside) and CLA-8 (α,β Lactose octa para-iodobenzoate) in SuBen and LOIB gels. The integrity of the CLA-8 gels was in addition investigated for 3 weeks using HPLC.

### Methods

The gels were prepared as described in Example 19.

HPLC assay: Samples were analyzed using a Shimadzu Nexera-i instrument. The samples were injected (5 μL) onto a Waters Terra RP8 column (5 μm, 4.6×150 mm, temperature 40° C.) at a flow rate of 1 mL/min. The solvent system consisted of mobile phase A (5% MeCN, 0.1% TFA in water) and mobile phase B (0.1% TFA in MeCN). Chromatographic separation was achieved using a gradient of 40 to 100% phase B in 10 min. Ultraviolet detection at 280 nm was used to identify the CT contrast agents. Gel samples were prepared by dilution of 50 μL gel in 1 mL MeCN, which were then diluted 10-fold in MeCN.

### Results and Discussion

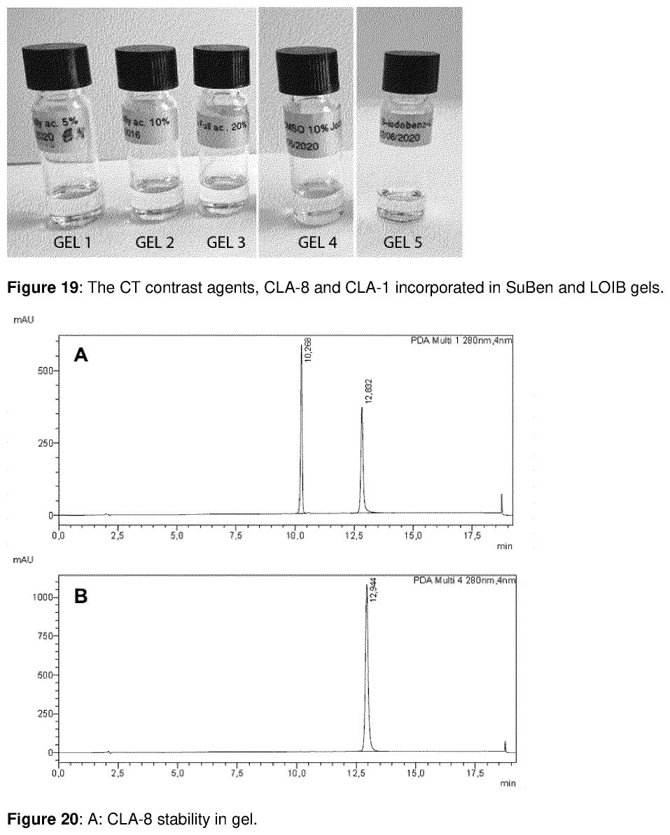

SuBen and LOIB-based gels were mixed according to table 9. CLA-8 was completely solubilized in SuBen gels up to 20% (Gel 3, table 9), and up to 10% in LOIB gels (Gel 4, table 9). The solubilized gels are shown in FIG. 19.

CLA-1 formed a fully transparent and homogeneous gel solution at 82.5% content (FIG. 19, Gel 5). Hence, CLA-1 may replace LOIB in formulations where contrast agents are beneficial.

TABLE 9
Compositions of gels containing CLA-1 or CLA-8. Composition are
given as weight percentage. The gels appearance is reported.

| Chemical | Gel 1 | Gel 2 | Gel 3 | Gel 4 | Gel 5 |
|---|---|---|---|---|---|
| SuBen | 55 | 50 | 40 | | |
| LOIB | | | | 65 | |
| CLA-1 | | | | | 82.5 |
| CLA-8 | 5 | 10 | 20 | 10 | |
| GTO | 25 | 25 | 25 | 10 | 7.5 |
| EtOH | 15 | 15 | 15 | | 10 |
| DMSO | | | | 15 | |
| Appearance | Clear | Clear | Clear | Clear | Clear |
| | gel | gel | gel | gel | gel |

Following preparation, Gel 3 and Gel 4 were sampled for HPLC and the gels were stored at 37° C. for 3 weeks, and then resampled for HPLC analysis. CLA-8 was identified as a single peak in the chromatograms at TO and after 3 weeks for both Gel 3 and Gel 4 indicating high integrity of CLA-8. Examples of chromatograms are shown in FIG. 20.

### Conclusion

SuBen and LOIB can incorporate 10-20% CLA-8 contrast agents in fully solubilised homogeneous gels. Homogeneous and fully solubilized CLA-1 gels can be produced with CLA-1 as the main carbohydrate ester component. CLA-8 was found to have high integrity in both LOIB and SuBen gels.

### Example 23: CT Contrast Estimates of Gel Formulations Containing CLA-1 or CLA-8

The iodine content of gels comprising CLA-8 or CLA-1 is calculated, and their CT contrast is estimated via comparison of their iodine content relative to the Lipiodol gels presented in example 8.

### Materials and Methods

Lipiodol contains 480 mg l/mL and has a density of 1.28 g/mL. This corresponds to 375 mg 1/g and hence a 37.5% w/w of iodine in Lipiodol. CLA-8 has 8 iodine and has a molecular weight of 2180 g/mol, corresponding to 46.6% w/w iodine. CLA-1 has 1 iodine and has a molecular weight of 1048 g/mol, corresponding to 12% w/w iodine. The % w/w iodine in a given gel formulation is determined as the weigh fraction of e.g. CLA-1, CLA-8 or Lipiodol in the formulation, and the iodine content of e.g. CLA-1, CLA-8 or Lipiodol. Iodine contents of the formulations presented in example 8 and in FIG. 7 are given in table 10.

### Results and Discussion

The iodine content of CLA-8 equals 8*Mw (I)/Mw (CLA-8)=46.6%. The iodine content of CLA-1 equals 1*Mw (I)/Mw (CLA-1)=12.1%. Mw is the molecular weight. A formulation containing 82.5% CLA-1 thus has a total iodine content of 82.5%×12%=10.0% w/w iodine.

TABLE 10
The iodine content is given as % iodine (w/w) for the lipiodol
gel formulations presented in example 8 and the CLA-1 and CLA-8
gel formulations described in example 22. The CT contrast for
the CLA-1 and CLA-8 formulations were estimated by linear regression
of the Lipiodol (CT (HU) = 300 × % Iodine (w/w))

| | | Mean CT |
|---|---|---|
| Formulation | % Iodine (w/w) | contrast (HU) |
| LOIB:Lipiodol:EtOH (75:15:10) | 5.6 | 1700 * |
| SuBen:Lipiodol:EtOH (60:25:15) | 9.4 | 2800 * |
| LOIB:CLA-8:EtOH (70:10:20) | 4.7 | 1400 ** |
| SuBen:CLA-8:EtOH (60:15:15) | 7.0 | 2100 ** |
| CLA-1:GTO:EtOH (82.5:7.5:10) | 10.0 | 3000 ** |

* Obtained from FIG. 7, example 8.
** CT contrast was estimated by linear regression using the lipiodol data.

### Conclusion:

The CLA-1 and CLA-8 gel formulations described in example 22 have comparable iodine content as the Lipiodol gels presented in examples 8, and thus have similar CT contrast.

### Example 24: Transesterification of Resiquimod and Gardiquimod in LOIB Gels

The aim of the current example is to investigate the stability of resiquimod and gardiquimod, when formulated in LOIB gels.

### Materials and Methods

Preparation of gels: Resiquimod (R848) and gardiquimod were formulated in LOIB gels (LOIB:GTO:ETOH 82.5:7.5:10) as described in example 19. Formulations were done in duplicates and had a final drug concentration of 1.2 mg/g.

Stability test: The gels were then incubated at 37° C. At fixed time points, 50 μL of gel were taken and dissolved in 1 mL acetonitrile (MeCN).

HPLC assay: Samples were analyzed using an UHPLC instrument (Shimadzu). The samples were injected (5 μL) onto a Waters Terra RP18 column (5 μm, 4.6×150 mm, temperature 40° C.) at a flow rate of 1 mL/min. The solvent system consisted of mobile phase A (5% MeCN, 0.1% TFA in water) and mobile phase B (0.1% TFA in MeCN). Chromatographic separation was achieved using a gradient of 0 to 100% phase B in 15 min. Ultraviolet detection at 260 nm and 320 nm was used to identify the drug compounds.

LC-MS assay: Resiquimod gel samples were analyzed in an ACQUITY UPLC instrument coupled with a QDa detector. Samples (5 μL) were injected onto a Waters BEH C18 column (2.1 μm, 2.1×50 mm, temperature 40° C.) at a flow rate of 0.4 mL/min. The solvent system consisted of mobile phase A (5% MeCN, 0.1% FA in water) and mobile phase B (0.1% FA in MeCN). Chromatographic separation was achieved using a gradient of 0 to 100% phase B in 6 min.

### Results and Discussion

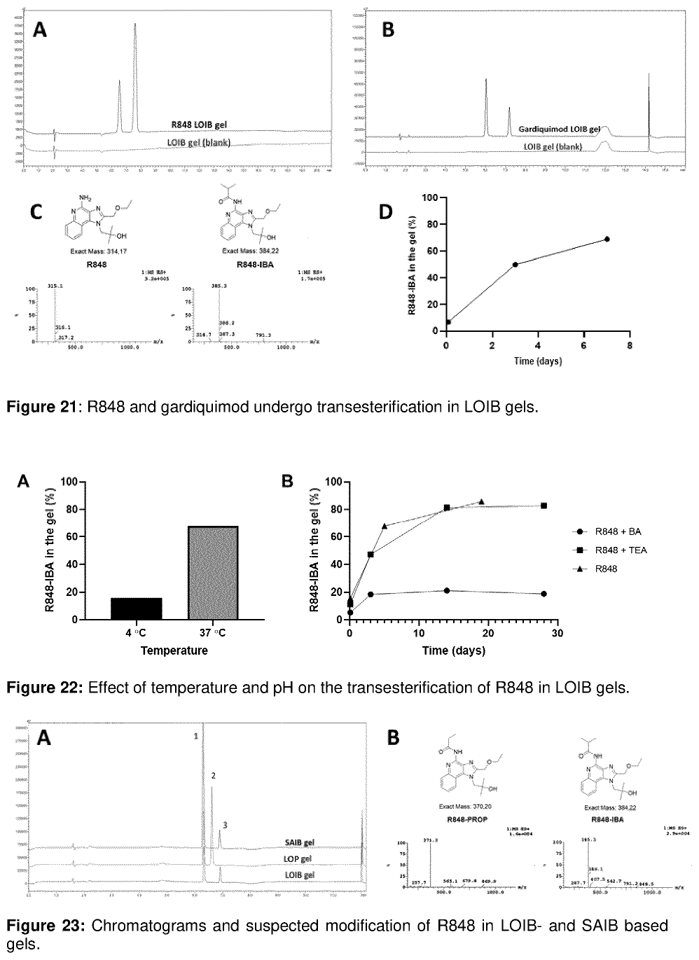

The HPLC chromatograms showed the presence of two different drug compounds in the R848 LOIB gels (FIG. 21A). The LC-MS analysis showed that the m/z values of such drug compounds were 385.2 and 371.3, which correspond to the mass+H values of R848 and a modified R848 product from a transesterification, respectively.

The transesterification causes the modification of the primary amine of R848 with isobutyric acid, forming the compound shown in FIG. 21C. Overtime R848 gradually converts to R848-isobutyrate in the LOIB gels (FIG. 21D).

An in-vitro release study was done to investigate if gardiquimod is prone to transesterification and if the product of such reaction can be released from the gel. Gardiquimod LOIB gels were injected (50 μL) in glass vials containing 2 mL of PBS and then incubated at 37° C. At fixed time points, 1 mL of the release media was taken and replaced with 1 mL of fresh PBS. The samples were analysed by HPLC (0-50% B in 7 min) and LC-MS.

As seen with R848, the HPLC chromatograms showed the presence of two different drug compounds in the release media from gardiquimod LOIB gels (FIG. 21B). The LC-MS analysis showed that the m/z values of such drug compounds were 314.2 and 384.2, which correspond to the mass+H values of gardiquimod and a modified gardiquimod product from a transesterification, respectively.

### Conclusion

Resiquimod (R848) and gardiquimod display poor stability in LOIB gels, and are found to undergo transesterification. In this process, the primary amine of Resiquimod and Gardiquimod is modified with isobutyric acid as shown in FIG. 21.

### Example 25: Effect of Temperature and pH on the Transesterification of R848

The aim of the current example is to show the effect of temperature and pH on the transesterification of resiquimod (R848) when formulated in LOIB gels.

### Materials and Methods

Preparation of resiquimod gels to test the effect of temperature: LOIB:GTO:EtOH (82.5:7.5:10 (% w/w)) with a concentration of 1.2 mg/g R848 was prepared as described in example 19. The gel was divided in individual glass vials, which then were incubated at either 4° C. or 37° C. After 5 days, 50 μL of gel were taken and dissolved in 1 mL MeCN.

Preparation of resiquimod gels to test the effect of pH: LOIB:GTO:EtOH (82.5:7.5:10 (% w/w)) gels with a concentration of 1.2 mg/g R848 were prepared as described in example 19. Then three gel formulations were obtained by adding either benzoic acid (BA), triethylamine (TEA) or none. The gels were incubated at 37° C. At fixed time points, 50 μL of gel were taken and dissolved in 1 mL MeCN.

HPLC assay: All samples were analyzed using an HPLC instrument (Shimadzu Nexera-i). The samples were injected (5 μL) onto a Waters Terra RP8 column (5 μm, 4.6×150 mm, temperature 25° C.) at a flow rate of 0.8 mL/min. The solvent system consisted of mobile phase A (5% MeCN, 0.1% TFA in water) and mobile phase B (0.1% TFA in MeCN). Chromatographic separation was achieved using a gradient of 0 to 100% phase B in 15 min. Ultraviolet detection at 320 nm was used to identify the drug compounds.

### Results and Discussion

After 5 days, around 15% of R848-isobutyrate (R848-IBA) is present in the gels incubated at 4° C. In contrast, approximately 70% of R848 is converted to R848-IBA in the same period at 37° C. (FIG. 22A). Thus at 4° C. there is approximately a 5-fold reduction in the conversion rate of resiquimod.

Acidic conditions shift the equilibrium of the R848 transesterification reaction. In the presence of BA, such reaction reaches equilibrium after 3 days, when the percentage of R848-IBA is around 18%. Contrastingly, in both a regular gel and in a gel with TEA (basic conditions), equilibrium is not reached until day 14 with around 80% of R848-IBA present in the gel (FIG. 22B).

### Conclusion

The conversion rate of R848 to R848-IBA can be decreased almost 5-fold by decreasing temperature from 37° C. to 4° C. The equilibrium of the transesterification reaction of R848 is shifted towards a lower degree on conversion in acidic conditions.

### Example 26: Effect of the Carbohydrate Ester Chemistry on the Transesterification of Resiquimod

The aim of the current examples is to investigate the conversion of Resiquimod into R848-IBA (isbutyrate-R848) in gels comprising carbohydrate esters based on non-reducing sugars, as well as propionate, isobutyrate or benzoate carbohydrate ester derivatives of lactose.

### Materials and Methods

Preparation of gels: gels with composition (% w/w) LOIB:EtOH (82.5:17.5), LOIB:GTO:EtOH (82.5:7.5:10), LOP:GTO:EtOH (82.5:7.5:10), SAIB:GTO:EtOH (82.5:7.5:10), SOIB:GTO:EtOH (80:10:10), ROIB:GTO:EtOH (80:10:10), TOIB:GTO:EtOH (80:10:10), SuBen:GTO:EtOH, (60:25:15), RaBen:GTO:EtOH, (60:25:15), LacBen:GTO:EtOH, (60:25:15), containing 1 mg/g Resiquimod were prepared according to example 19. The gels were then stored at 37° C., and 50 μL samples diluted in 1 ml MeCN were taken as function of time for HPLC analysis.

HPLC assay: HPLC analysis was conducted on a Shimadzu Nexera-i system. The samples were injected (5 μL) onto a Waters Terra RP8 column (5 μm, 4.6×150 mm, temperature 25° C.) at a flow rate of 0.8 mL/min. The solvent system consisted of mobile phase A (5% MeCN, 0.1% TFA in water) and mobile phase B (0.1% TFA in MeCN). The gradient was 0 to 100% phase B in 15 min. Ultraviolet detection at 320 nm was used to identify the drug compounds.

### Results and Discussion

All gel samples were successfully prepared and HPLC samples were taken as function of time. The conversion of Resiquimod was studied for all gels, and conversion products were identified by LC-MS. The conversion products formed in LOP (lactose octa-propionate), SAIB (sucrose acetate isobutyrate) and LOIB (lactose octa-isobutyrate) are shown in FIG. 23.

The conversion of Resiquimod in gels containing LOP or LOIB are presented in table 11. The conversion of Resiquimod in gels containing SAIB or octaisobutyrate esters of Sucrose, Raffinose and Trehalose are presented in table 12. The conversion of Resiquimod in gels containing SuBen, LacBen or Raben are presented in table 13.

TABLE 11
Percent conversion of Resiquimod in gels given as function
of the incubation time at 37° C. Conversion is presented
for LOIB gel with and without the co-solvent GTO and for
a LOP:GTO gel. Gel compositions are given as weight ratio.

| Time | LOIB:EtOH | LOIB:GTO:EtOH | LOP:GTO:EtOH |
|---|---|---|---|
| (days) | (82.5:17.5) | (82.5:7.5:10) | (82.5:7.5:10) |
| 0.083 (2 h) | 6.8 | 15.6 | 5.0 |
| 1 | — | — | 30.6 |
| 3 | 50.1 | — | 58.9 |
| 5 | — | 67.7 | — |
| 7 | 69.7 | — | 76.0 |
| 14 | 78.5 | — | 82.1 |
| 19 | — | 85.7 | — |

TABLE 12
Percent conversion of Resiquimod in gels given as function of the
incubation time at 37° C. The conversion is compared for gels
based on SAIB and for the octaisobutyrate esters of Sucrose, Raffinose
and Trehalose. Gel compositions are given as weight ratio.

| Time | SAIB:GTO:EtOH | SOIB:GTO:EtOH | ROIB:GTO:EtOH | TOIB:GTO:EtOH |
|---|---|---|---|---|
| (days) | (82.5:7.5:10) | (80:10:10) | (80:10:10) | (80:10:10) |
| 0.083 (2 h) | 0.9 | 0.0 | 0.0 | 0.0 |
| 1 | 3.9 | 0.8 | 1.7 | 1.4 |
| 3 | 7.5 | — | — | — |
| 4 | — | 2.5 | 5.1 | 4.2 |
| 7 | 10.3 | — | — | — |
| 14 | 12.4 | — | — | — |

TABLE 13
Percent conversion of Resiquimod in gels given as function of the incubation
time at 37° C. Gel compositions are given as weight ratio.

| Time | SuBen:GTO:EtOH | RaBen:GTO:EtOH | LacBen:GTO:EtOH |
|---|---|---|---|
| (days) | (60:25:15) | (60:25:15) | (60:25:15) |
| 0.083 (2 h) | 0.0 | 0.0 | — |
| 1 | 0.0 | 0.0 | — |
| 3 | 0.0 | 0.0 | — |
| 7 | 0.0 | 0.0 | — |
| 14 | 0.0 | 0.0 | — |
| 60 | — | — | 0.0 |

High degree of conversion of Resiquimod is observed for LOIB gels independent of GTO. In LOIB gels, Resiquimod is found to convert into R848-IBA (FIG. 23B, Table 11). An equally high conversion of Resiquimod is found for LOP based gels, where Resiquimod is converted into R848-PROP (FIG. 23B, Table 11).

LOP and LOIB are the octapropionate and octaisobutyrate ester derivatives of Lactose respectively, and Lactose is a reducing sugar containing one anomeric centre. To investigate the effects related to the anomeric centre of Lactose, conversion of Resiquimod was investigated in gels comprising the octaisobutyrate ester derivatives of Sucrose (SOIB), Raffinose (ROIB), and Trehalose (TOIB). Conversion of Resiquimod was further investigated in gels comprising the mixed acetate isobutyrate derivative of Sucrose, SAIB.

In comparison to LOIB gels, a significant reduction in the conversion of Resiquimod of approximately 10-fold was found for gels based on the derivatives SOIB, ROIB and TOIB. Gels based on non-reducing carbohydrates (Sucrose, Raffinose and Trehalose) with no anomeric centre thus display lower reactivity in transesterification reactions with Resiquimod (Table 12). In comparison to SOIB (Sucrose octaisobutyrate), SAIB (Sucrose acetate isobutyate) displayed higher reactivity in the transesterification reaction with Resiquimod. Overall, the conversion of Resiquimod in SAIB gels was significantly reduced compared to LOIB gels but had a 2-3 fold higher conversion than for SOIB, ROIB and TOIB gels (Table 12).

Conversion of Resiquimod was further analysed in gels based on octabenzoate esters of Sucrose, Lactose and Raffinose (SuBen, LacBen and RaBen). No conversion of Resiquimod was found in neither the SuBen, LacBen nor RaBen based gels (Table 13).

### Conclusion

The presence of an anomeric centre in LOIB was shown to increase the reactivity towards transesterification with Resiquimod. Octaisobutyrate esters of non-reducing carbohydrates (Sucrose, Raffinose and Trehalose, without an anomeric centre) were found to have reduced reactivity towards transesterification with Resiquimod. No reactivity towards transesterification with Resiquimod was detected for gels based on SuBen, LacBen or RaBen. SAIB with mixed acetate and isobutyrate esters displayed higher reactivity towards transesterification with R848 compared to the octaisobutyrate variant SOIB.

### Example 27: Methoxy-LOIB (meLOIB) Gels Display Highly Reduced Reactivity Towards Transesterification of Resiquimod

The aim of the current example was to investigate the transesterification of Resiquimod in gels comprising methoxy-LOIB (meLOIB).

### Materials and Methods

Preparations of gels: A meLOIB:GTO:EtOH (82.5:7.5:10 (% w/w)) gel containing 1 mg/g Resiquimod was prepared according to example 19. The synthesis of meLOIB is described in example 36.

The gel was stored at 37° C. and samples were taken for HPLC analysis over time. 50 μL samples were taken and diluted in 1 mL MeCN for HPLC analysis.

HPLC assay: HPLC analysis was conducted on a Shimadzu Nexera-i system running a 5% MeCN+0.1% TFA in MQ water (solvent A) and MeCN+0.1% TFA (solvent B) as mobile phase with a Waters Terra RP8 column (5 μm, 4.6×150 mm). The flow rate was 0.8 ml/min, injection volume of 5 μL, temperature 25° C. The gradient was 0-100% B in 15 min.

### Results and Discussion

meLOIB gel containing Resiquimod was transparent and homogeneous after preparation. Then the gel was stored at 37° C., and the transesterification of Resiquimod was assayed by HPLC.

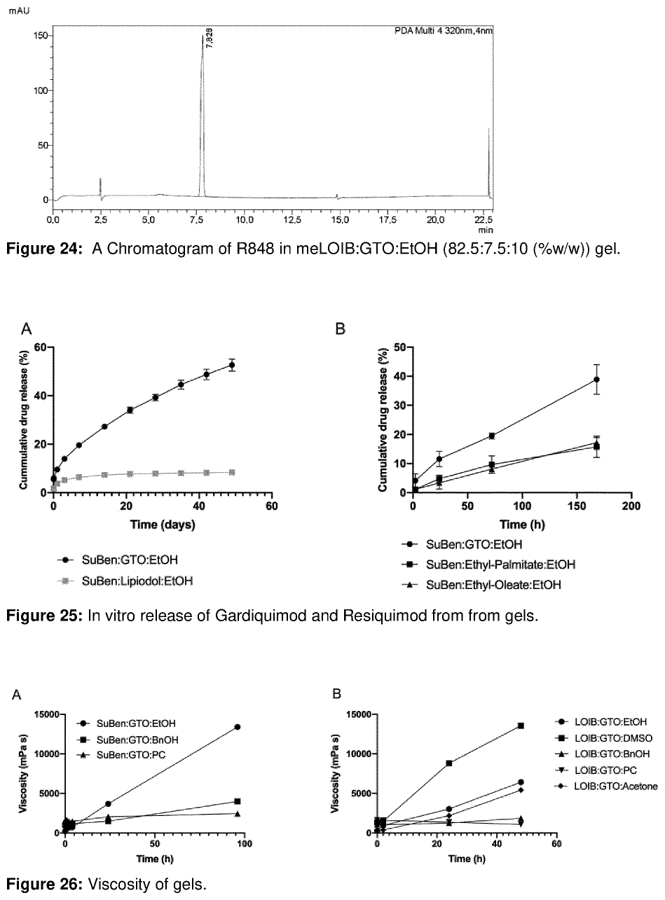

The meLOIB gel was sampled at Oh, 3 days, 7 days and 2 months and no transesterification of Resiquimod was observed at any timepoint. All chromatograms were identical to the chromatogram displayed in FIG. 24.

### Conclusion

Substituting the isobutyric acid on the anomeric centre of LOIB with a methoxy group abolishes LOIB reactivity with Resiquimod. No transesterification is observed in meLOIB gels within the timeframe of the experiment.

### Example 28: Lipiodol, Ethyl-Palmitoyl and Ethyl-Oleate Ester Reduces the Release of API from Gels

In the current example the release of Resiquimod and Gardiquimod are investigated for gels containing Lipiodol or ethyl-esters as co-solvent. Gels comprising GTO were included as reference.

### Materials and Methods

Preparation of Gardiquimod gel: SuBen:GTO:EtOH (60:25:15 (% w/w)) and SuBen:Lipiodol:EtOH (60:25:15 (% w/w)) were prepared by simple mixing and sonication (80° C., 90-120 min) as described in example 19. Stocks containing 1.2 mg/g Gardiquimod were prepared from each gel (GTO or Lipiodol gel) by adding 1.25 g of gel on top of 1.5 mg Gardiquimod followed by stirring.

Preparation of Resiquimod gel: SuBen:GTO:EtOH:PLA (60:25:15:0.5 (% w/w)), SuBen:Ethyl-palmitate:EtOH (60:25:15 (% w/w)), and SuBen:Ethyl-oleate:EtOH (60:25:15 (% w/w)) were prepared by simple mixing and sonication (80° C., 90-120 min) as described in example 19. Stocks containing 1.2 mg/g Resiquimod were prepared from each gel by adding 1.25 g of gel on top of 1.5 mg Resiquimod followed by stirring.

Release assay: 2 mL of PBS buffer is pipetted into an 8-mL glass vial. 100 μL of gel is injected into the buffer, and the injected mass is quantified by a scale. The vials are closed with lids and are incubated at 37° C. At each sampling point, take 1 mL of buffer and place it inside an Eppendorf tube. Then add 1 mL of fresh buffer to replace the taken sample. The samples are analyzed by HPLC.

HPLC assay: Gardiquimod and Resiquimod were quantified in a Shimadzu instrument using a Waters Terra C18 or C8 column (both 5 μm, 4.6×150 mm), respectively. For both compounds, the flowrate was 1 mL/min and the column temperature was 40° C. For gardiquimod, the injection volume was 30 μL and the gradient was 0-50% B over 7 min with the mobile phases as 5% MeCN+0.1% TFA in MQ water (A) and MeCN+0.1% TFA (B). For resiquimod, the injection volume was 40 μL and the gradient was 0-60% B over 10 min with the mobile phases as 5% MeCN+0.1% TFA in MQ water (A) and MeCN+0.1% TFA (B).

### Results and Discussion

Suben gels containing either Lipiodol or GTO were successfully prepared and Gardiquimod was formulated at 1.2 mg/g. Release studies were conducted and release media samples were analysed by HPLC. The Gardiquimod release data are presented in FIG. 25A.

Suben gels containing either Ethyl-palmitate or Ethyl-oleate were successfully prepared and Resiquimod was formulated at 1.2 mg/g in gels. Release studies were conducted and release media samples were analysed by HPLC. The Resiquimod release data are presented in FIG. 25B.

The in vitro release of Gardiquimod from a SuBen gel is reduced upon substitution of the co-solvent GTO with Lipiodol (FIG. 25A). At the timepoint 49 days, the release of Gardiquimod is reduced 6-fold from 52.5% to 8.4% (FIG. 25A).

The in vitro release of Resiquimod from a SuBen gel is reduced upon substitution of the co-solvent GTO with Ethyl-palmitate or Ethyl-oleate (FIG. 25B). At the 7 day timepoint, the average release of Resiquimod is reduced 2.5-fold from −39% to 15% (FIG. 25B).

### Conclusion

Substitution of the co-solvent GTO with either Lipiodol, Ethyl-palmitate or Ethyl-oleate reduces the in vitro release of Gardiquimod or Resiquimod from SuBen gels 2.5-6 fold.

### Example 29: Viscosity of Gel Formulation with and without Solvent

The aim of the current example is to investigate the change in viscosity of SuBen and LOIB gels upon efflux of the solvent EtOH. The viscosity of the gels was determined before and after evaporation of EtOH using a vacuum oven.

### Materials and Methods:

Preparation of gels: SuBen:GTO:EtOH:PLA (60:25:15:0.5 (% w/w)), LOIB:GTO:EtOH (82.5:7.5:10 (% w/w)), and LOIB:GTO:EtOH (75:15:10 (% w/w)) gels were prepared in 6 gram scale according to example 19.

Viscosity measurement: 1 mL of gel was pipetted into an EMS-1000 viscometer tube and a 4.7 mm sphere was added. The sample tube was then sealed with a lid to prevent evaporation. The sample viscosity was determined using standard settings of 1000 rpms, 5 s to 45 min sampling time depending on temperature and viscosity. Viscosities were determined at 25, 35, 37, 40, 60° C. for sample containing EtOH and 35, 37, 40, 60, 80° C. for sample without EtOH.

Removal of solvent: The EMS-1000 tube sample lid was removed and the tube containing sphere and gel was incubated at 120° C. and at reduced pressure for 16 hours. The sample was equilibrated at room temperature before measurement of the viscosity.

### Results and Discussion

The gels were prepared and the viscosity was determined as function of temperature. Then, ethanol was evaporated from the gels using a vacuum oven, and the viscosity of the gel samples were determined as function of temperature. The compiled results are given in table 14.

TABLE 14
The viscosity (mPa s) of SUBEN = SuBen:GTO:EtOH:PLA (60:25:15:0.5
(% w/w)), LOIB7.5 = LOIB:GTO:EtOH (82.5:7.5:10 (% w/w)),
and LOIB15 = LOIB:GTO:EtOH (75:15:10 (% w/w)) gels are given
as function of temperature, and with (+EtOH) and without (−EtOH)
ethanol. Viscosities were determined using an EMS-1000 viscometer.

| | SUBEN + | SUBEN − | LOIB7.5 + | LOIB7.5 − | LOIB15 + | LOIB15 − |
|---|---|---|---|---|---|---|
| Temperature | EtOH | EtOH | EtOH | EtOH | EtOH | EtOH |
| 25° C. | 740 | — | 1320 | — | 478 | 243 000 |
| 35° C. | 346 | 307 000 | 525 | — | 225 | 42800 |
| 37° C. | 291 | 209 000 | 455 | 1 320 000 | 191 | 31200 |
| 40° C. | 228 | 121 000 | 352 | 676 000 | 156 | 20 650 |
| 60° C. | 62 | 6400 | 93 | 19000 | 50 | 1880 |
| 80° C. | — | 860 | 34 | 1725 | 20 | 351 |

The viscosity of SuBen:GTO:EtOH:PLA (60:25:15:0.5 (% w/w)) was found to decrease with increasing temperature both with and without EtOH (Table 14). At 37° C. the viscosity increased more than 700-fold upon removal of ethanol. Upon injection of the gel into tissue the solvent will diffuse out and an increase of 700-fold in viscosity can be expected.

The viscosity of both LOIB formulations was found to decrease with increasing temperature both with and without EtOH (Table 14). The change in co-solvent from 15% GTO to 7.5% GTO resulted in an increase from 31200 to 1320000 mPa s for the LOIB gels at 37° C. Thus, the co-solvent content can be used effectively to control the viscosity of the formed depot.

### Conclusion

The viscosity of a SuBen gel was found to decrease as function of temperature and display a more than 700-fold increase in viscosity upon solvent removal. Likewise, the LOIB gel containing 7.5% GTO displayed a 2900-fold increase in viscosity upon solvent removal.

### Example 30: Solvent Dependent Increase in Viscosity for SuBen or LOIB Gels

The aim of the current example is to investigate the increase in viscosity of LOIB and Suben gels upon solvent efflux. The increase in viscosity will be investigated for a range of solvents with different hydrophilicities, including Ethanol (EtOH), DMSO, propylene carbonate (PC), acetone, and benzyl alcohol (BnOH).

### Materials and Methods

Preparation of LOIB gels: LOIB gels with the compositions, LOIB:GTO:EtOH (80:10:10 (% w/w)), LOIB:GTO:DMSO (80:10:10 (% w/w)), LOIB:GTO:BnOH (80:10:10 (% w/w)), LOIB:GTO:PC (80:10:10 (% w/w)), and LOIB:GTO:Acetone (80:10:10 (% w/w)) were prepared in 2 gram scale as described in example 19.

Preparation of SuBen gels: LOIB gels with the compositions, SuBen:GTO:EtOH (60:25:15 (% w/w)), SuBen:GTO:BnOH (60:25:15 (% w/w)), and SuBen:GTO:PC (60:25:15 (% w/w)) were prepared in 2 gram scale as described in example 19.

Viscosity measurements: 500 μL of gel is injected using a 21 G needle in the EMS-1000 viscometer tubes containing a 4.7 mm diameter sphere. The viscosity at time 0 and following measurements were determined at 37° C. 5 mL of PBS is carefully injected on top of the gels with a slightly tilted tube and the tubes are sealed with a cap to eliminate evaporation. The tubes are incubated at 37° C. in between measurements to mimic solvent efflux at body temperature. For LOIB gels, viscosity measurements were performed after preparation, 2 h, 24 h and 48 h post addition of PBS. For SuBen gels, viscosity measurements were performed after preparation, 0.5 h, 1 h, 2 h, 4 h, 24 h and 96 h post addition of PBS. 4.5 ml of PBS is exchanged every measurement to ensure that the sink contains small amounts of solvent. Measurements are performed as doublets.

### Results and Discussion

SuBen and LOIB gels were prepared and injected into the EMS-1000 viscometer tubes and the initial viscosity was determined at 37° C. Then a sink volume of 5 mL PBS was added to each tube in order to induce efflux of the water-soluble solvents. The viscosity was determined as function of incubation time at 37° C. and the results are presented in FIG. 26.

In SuBen gels, EtOH showed the fastest increase in viscosity, whereas the viscosity of SuBen gels containing BnOH and PC remained unchanged indicating no or little release of BnOH or PC (FIG. 26A).

In SuBen gels, the initial viscosity followed the order Suben:GTO:EtOH (220 mPa s), Suben:GTO:BnOH (1020 mPa s) and Suben:GTO:PC (1570 mPa s)

For LOIB gels, DMSO showed the fastest increase in viscosity followed by EtOH and Acetone. The viscosity of LOIB gels containing PC or BnOH did not increase over the experimental time of 48 h indicating no or little release of these solvents. The rate in viscosity increase and thereby thus followed the sequence: DMSO>EtOH˜Acetone>BnOH˜PC (FIG. 26B).

For LOIB gels, the initial viscosity followed the sequence LOIB:GTO:Acetone (183 mPa s), LOIB:GTO:EtOH (275 mPa s), LOIB:GTO:BnOH (1070 mPa s), LOIB:GTO:DMSO (1335 mPa s) and LOIB:GTO:PC (1630 mPa s).

### Conclusion

SuBen:GTO:EtOH and LOIB:GTO:DMSO gels displayed the fastest and highest increase in viscosity.

DMSO induced a faster and higher increase in viscosity compared to EtOH for LOIB gels. Gels containing PC and BnOH showed little or no change in viscosity for both LOIB and SuBen gels. The solvent can thus be used for tuning of the gels initial viscosity, and the rate of increase in gel viscosity upon injection into aqueous media or tissue. Gels incorporating EtOH or DMSO may therefore settle (form a viscous solution) faster than gels containing PC or BnOH when injected into tissue.

The contact area between the gel and PBS sink volume is, for the current tube setup, much smaller than when injected into tissue. A faster increase in viscosity is therefore expected in tissue.

### Example 31: Biodistribution and Pharmacokinetics of Tritiated R848 after Intratumoral and Intravenous Injection as Free R848 and Injection of R848 Incorporated in LOIB:GTO:EtOH (82.5:7.5:10 (% w/w)) Gel

The aim of the current example is to investigate the biodistribution and pharmacokinetics of tritiated R848 injected intravenously and intratumorally as free drug compared to intratumoral injection of tritiated R848 in LOIB:GTO:EtOH (82.5:7.5:10) gel.

### Materials and Methods

Three preparations were prepared consisting of [³H]R848 gel (LOIB:GTO:EtOH (82.5:7.5:10 (% w/w))) and [³H]R848 in phosphate buffered saline (PBS) for either intravenous (IV) or intratumoral (IT) administration. [³H]R848 was purchased from Hartmann Analytic (Germany) as a solution in ethanol. The gel was prepared by placing the [³H]R848 stock (330 μL, 12 MBq) in a glass vial and removing the ethanol under a stream of argon for 10 min. R848 gel (1.2 mg/mL R848, LOIB:GTO:EtOH (82.5:7.5:10 (% w/w)), 1.71 g, 1.62 mL) was then added to the dry residue and the mixture was magnetically stirred at 30° C. for 30 min. After complete stirring, the entire mixture was transferred to a glass vial, resulting in a transfer of 1.68 g (1.59 mL), furnishing the final [³H]R848 gel. To test the homogeneity and radioactivity concentration of the LOIB:GTO:EtOH (82.5:7.5:10 (% w/w)) gel, two samples of 36.2 mg (34.5 μL) and 28.2 mg (26.9 μL) were removed. The samples were diluted with ethanol to 5.00 mL and from each diluted solution, 200 μL was added to a scintillation vial containing 10 mL Ultima Gold. The two [³H]R848 solutions in PBS were prepared by drying down the [³H]R848 stock (459 μL, 18 MBq) as above. To the dry residue was then added a solution of R848 in PBS (1.2 mg/mL, 2.40 mL). The mixture was stirred for 1 hour at 40° C. From the resulting mixture, 1.22 mL was collected for IT injections (1.2 mg/mL [³H]R848 in PBS). From the same mixture, 1.10 mL was collected and mixed with 1.10 mL PBS, to generate the IV injection sample (0.6 mg/mL [³H]R848 in PBS). From each of the PBS solutions, 2×10 mL was removed to test for homogeneity and radioactivity concentration. All the samples were diluted to 1.00 mL with ethanol and 200 μL was added to a scintillation vial containing 10 mL Ultima Gold.

8-9 week old female BALB/c mice were inoculated with 3×10⁵ CT26 tumor cells in 100 μL of RPMI on the right flank by subcutaneous injection on day 0. When the average tumor size reached approximately 140 mm³ (day 13), mice were distributed into fifteen groups based on a size dependent randomization (n=4 per group). Mice were injected with one dose of [³H]R848 administered either IV in PBS (3.75 MBq/mL [³H]R848, 3 mg/kg [³H]R848), IT in PBS (7.5 MBq/mL [³H]R848, 3 mg/kg [³H]R848) or IT with [³H]R848 gel (LOIB:GTO:EtOH (82.5:7.5:10 (% w/w))) as previously described (7.4 MBq/mL [³H]R848, 3 mg/kg [³H]R848). At multiple time points tissue and blood samples were collected (blood: 15 min, 30 min, 1 hour, 3 hours, 6 hours, 24 hours, 72 hours, 1 week; tissues: 30 min, 3 hours, 24 hours, 72 hours, 1 week). The samples were dissolved in Soluene 350 and/or EtOH (1-6 mL) and heated overnight at 60° C. For decolorization, 0.2 mL hydrogen peroxide (30%) was added to the samples and they were heated for 30 min at 60° C. 200 μL sample solution was transferred to liquid scintillation vials together with 10 mL Ultima Gold. Scintillation vials, Ultima Gold and Soluene 350 were purchased from Perkin Elmer (USA). Tritium content was measured by liquid scintillation counting on a Hidex 300 SL, using the tritium setting (Hidex, Finland). Prepared samples were allowed to settle for at least until the next day before counting and counted until 1600 counts or for 10 minutes. Counts-per-minute (CPM) values were used in all calculations.

### Results and Discussion

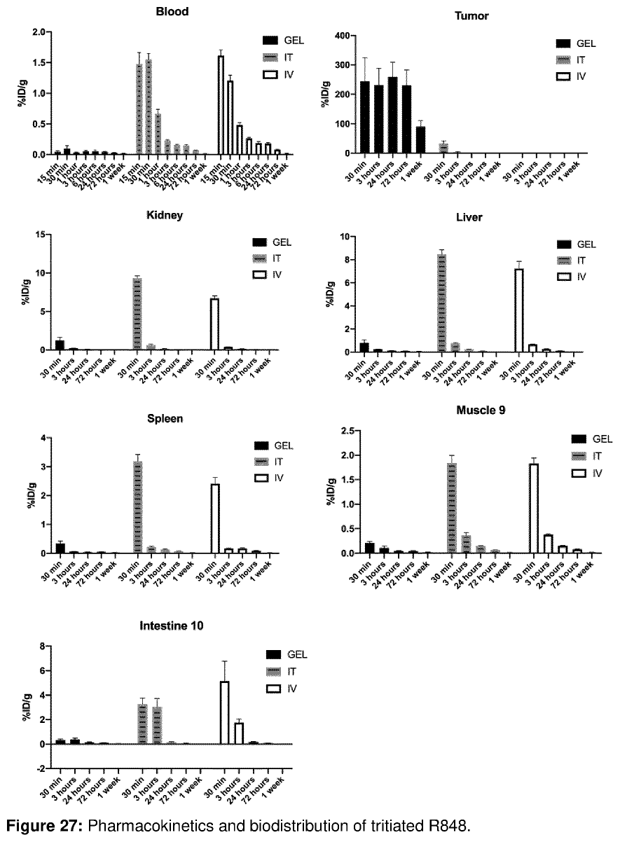

All [³H]R848 pharmacokinetics and biodistribution data were compiled and are presented in FIG. 27. [³H]R848 concentration in the blood was found to be very low at all time points evaluated in connection with intratumoral administration as [³H]R848 gel whereas much higher concentration was observed for the intravenous and intratumoral administration of [³H]R848 in PBS (FIG. 27). Interestingly, the blood concentration levels of [³H]R848 reached comparable levels after intratumoral and intravenous administration of [³H]R848 in PBS (FIG. 27A). These observations show that [³H]R848 gel induce a much lower systemic exposure to R848. The [³H]R848 gel injected tumors also display the highest degree of retention in the tumor compared to both intratumoral and intravenous administration of [³H]R848 in PBS (FIG. 27B). The retention of [³H]R848 in PBS after intratumoral injection is already highly reduced compared to activity of [³H]R848 gel at 30 minutes after intratumoral injection. For [³H]R848 in PBS injected intravenously, only very low concentration is observed in the tumor. The improved retention of [³H]R848 in the gel and tumor tissue compared to [³H]R848 in PBS is also evidenced by the much higher observed concentration of [³H]R848 in kidney, liver, spleen, muscle and intestines of the [³H]R848 in PBS injected groups (FIG. 27C-G).

### Conclusion

[³H]R848 gel (LOIB:GTO:EtOH (82.5:7.5:10 (% w/w))) display a much higher retention of [³H]R848 in tumors than after intratumoral or intravenous injection of [³H]R848 in PBS. The observed improved tumor retention results in a lower systemic exposure.

### Example 32: In Vivo Release of TGFbi from Gel Depots

The aim of the current example is to demonstrate sustained release of three different TGFb inhibitors (TGFβi) from gel depots implanted in mice.

### Materials and Methods

Gel formulations: SD-208 (8 mg/g) was formulated in a SuBen:GTO:EtOH (60:25:15 (% w/w)) gel according to the procedure described in Example 19. Repsox (8 mg/g) or Galunisertib (8 mg/g) were formulated in LOIB:GTO:EtOH (82.5:7.5:10 (% w/w)) gels according to the procedure described in Example 19.

Animal handling: For each formulation 50 μL of gel was injected intratumorally in female Balb/c mice with CT26 tumors (Initial tumor volume of approximately 200 mm³). The mice were sacrificed at different time points and gel samples were collected. The gels were stored at 4° C. until analysis. For each gel, four mice were sacrificed at each timepoint. Samples were collected at 1 hour, 3 hours, 6 hours, 12 hours, 24 hours, 3 days and 7 days after injection.

Sample preparation: The collected gel from mice and gel that had not been injected (reference) was dissolved in 1 mL DMSO (gels containing Galunisertib) or 1 mL DMSO:MeCN with 0.1% TFA (50:50) (gels containing RepSox or SD-208). Samples were heated to 50° C. and stirred at 600 rpm to promote dissolution. The samples were subsequently filtered through 0.45 μm pore, nylon syringe filters.

HPLC method: Samples were analyzed on a Shimadzu Nexera-i HPLC. Samples were injected (5 μL) onto a Waters Terra RP8 column (5 μm, 4.6×150 mm, temperature 40° C.) at a flow rate of 0.8 mL/min. The solvent system consisted of mobile phase A (5% MeCN, 0.1% TFA in water) and mobile phase B (0.1% TFA in MeCN). Chromatographic separation was achieved using an optimized gradient. For samples containing Galunisertib or RepSox: 0-60% phase B in 6 min, 60% phase B for 5 min, 60-100% phase B in 5 min and 100% phase B for 4 min. For samples containing SD-208: 0-100% phase B in 13 minutes and 100% phase B for 4 min. UV detection was used to measure the content of Galunisertib (320 nm), RepSox (320 nm), SD-208 (350 nm), LOIB (220 nm) and SuBen (280 nm). HPLC results were correlated to standard curves and for the RepSox and Galunisertib gels the release was calculated based on the TGFβi:LOIB ratio in the collected gel compared to the total TGFβi:LOIB ratio in the reference gel. For the SD-208 gels the release was calculated based on the TGFβi:SuBen ratio in the collected gel compared to the total TGFβi:SuBen ratio in the reference gel.

### Results and Discussion

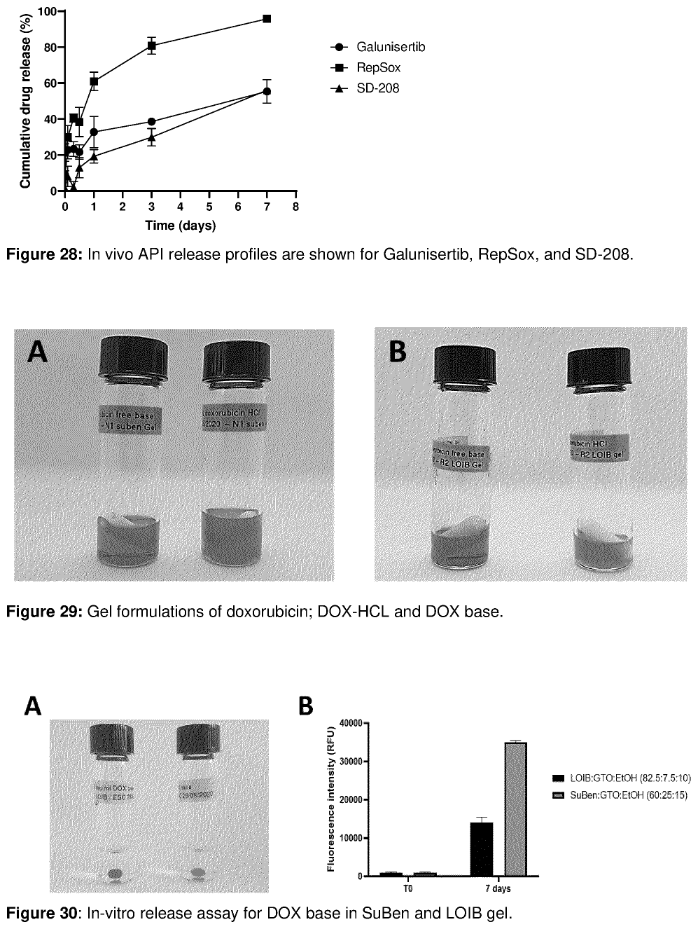

The in vivo release of SD-208, Galunisertib and Repsox was determined by ratiometric analysis, i.e. the API to SuBen or LOIB ratio was quantified by HPLC as function of time. Representative release profiles are shown in FIG. 28.

All three formulations display sustained release of the API over 7 days with RepSox having the fastest release, followed by Galunisertib and SD-208 with the lowest release (FIG. 28). The release rate is furthermore inversely correlated to the hydrophobicity of the API, and the most hydrophobic TGFb inhibitor (SD-208, log P=3.5) has the slowest release.

### Conclusion

The three TGFb inhibitors, SD-208, Galunisertib and RepSox were successfully formulated in gels and injected into mice. All TGFb inhibitors showed sustained release over the 7-day period evaluated. The release rate and total release over 7 days was found to be inversely correlated to the hydrophobicity of the TGFb inhibitor.

### Example 33: The Non-Salt Form of Doxorubicin has Higher Solubility in Gel

The aim of the current example is to show that the non-salt or base form of doxorubicin is more soluble in the gels, and that doxorubicin is released from the formulation.

### Materials and Methods

Conversion of DOX-HCl to its base form: Around 20 mg of doxorubicin-HCl (DOX-HCl) were dissolved in water (5 ml) and DCM (5 ml) was added. A few drops of sodium hydroxide in water (10M) were added until the water phase was basic (change in color to dark blue). Then doxorubicin was extracted with DCM (8×5 mL) and the combined organic phases were dried over magnesium sulfate and filtered. Finally, the solvent was removed in vacuum to obtain the free base of doxorubicin (DOX base) as a pink powder. To aliquot the powder, it was dissolved in DMSO:water (1:10) and freeze-dried for 48 h.

Preparation of gels: DOX base and DOX-HCl (0.8 mg/g) were formulated in a SuBen:GTO:EtOH (60:25:15 (% w/w)) gel according to the procedure described in Example 19. Also, DOX base and DOX-HCl (0.8 mg/g) were formulated in a LOIB:GTO:EtOH (82.5:7.5:10 (% w/w)) gel according to the procedure described in Example 19.

In-vitro release: 50 μL of DOX base SuBen:GTO:EtOH (60:25:15 (% w/w)) gel and 50 μL of LOIB:GTO:EtOH (82.5:7.5:10 (% w/w)) gel were injected in 8-mL glass vials containing 2 mL PBS. The vials were incubated at 37° C. After 7 days, 1 mL of PBS was taken and analysed by fluorescence spectroscopy.

Fluorescence spectroscopy: Fluorescence measurements were taken by a Tecan fluorescence microplate reader using 96-well, transparent Nunc plates. Sample volume was 200 μL. The excitation wavelength was 470 nm (5 nm bandwidth) and the emission wavelength was 595 nm (5 nm bandwidth).

### Results and Discussion

Both gel formulations (LOIB and SuBen) containing doxorubicin as a free base are clearer and more transparent, showing an improved solubility. In contrast, the opacity observed in the gels formulated with DOX-HCl indicates that micro-crystals are present, and that DOX-HCl is not solubilized (FIG. 29).

Both gel formulations containing DOX base are easily injected in PBS, which form spheres upon injection. After 7 days, there is an increase in fluorescence intensity in the release media from both DOX base SuBen:GTO:EtOH (60:25:15 (% w/w)) and LOIB:GTO:EtOH (82.5:7.5:10 (% w/w)) gels (FIG. 30A). This indicates that doxorubicin is present in the sample, thus the compound is released from the gels (FIG. 30B).

### Conclusion

Doxorubicin in base form is fully solubilized in LOIB and SuBen gels, producing transparent formulations. Contrary, the HCl salt of doxorubicin forms turbid unclear gel solutions. The non-salt or base form of doxorubicin is released from the gels.

### Example 34: Intratumoral Chemo-Immunotherapy Using Multi-Drug Releasing Gels

The aim of the current example is to demonstrate that combined chemo-immunotherapy delivered in an intratumoral gel formulation has therapeutic potential in murine cancer model.

### Methods

Preparation of chemo-immunotherapy gel: a LOIB:GTO:EtOH (82.5:7.5:10 (% w/w)) gel containing 1.0 mg/g Doxorubicin, 1.2 mg/g Resiquimod and 8 mg/g REPsox (RepSox) was prepared according to example 19. A LOIB:GTO:EtOH (82.5:7.5:10 (% w/w)) gel containing 1.0 mg/g Mitoxantrone, 1.2 mg/g Resiquimod and 8 mg/g REPsox (RepSox) was prepared according to example 19.

8 week-old female BALB/c mice were inoculated with 3×10⁵ CT26 tumor cells in 100 μL of RPMI on the right flank by subcutaneous injection on day 0. On day 14, inoculation tumors had an average size of 143.5 mm³ (SEM 13.6 mm³) and were divided into treatment groups based on a size-dependent randomization (n=6-8/group). Mice were treated with doxorubicin/REPsox/R848 chemo-immunotherapy gel, mitoxantron/REPsox/R848 chemo-immunotherapy gel or included as untreated controls. Chemo-immunotherapy gel was injected intratumorally using a 23 G needle and 1 ml syringe on mice anesthetized by inhalation anesthesia (˜4.5% sevoflurane). The injected chemo-immunotherapy gel volume was 50 μL for all injections. A total of three injections were performed with 7 day interval (Day 14, 21 and 28 after inoculation) during anesthesia (˜4.5% sevoflurane). Tumor and bodyweight were recorded 3 times per week. Mice were terminated from the study once their tumor reached a tumor volume 1000. Additional, study endpoints included weight loss >15% and presence of extensive ulcerations on the tumors. The tumors were measured by a digital caliper and the tumor volumes were calculated using the equation (length×width2)/2.

### Results and Discussion

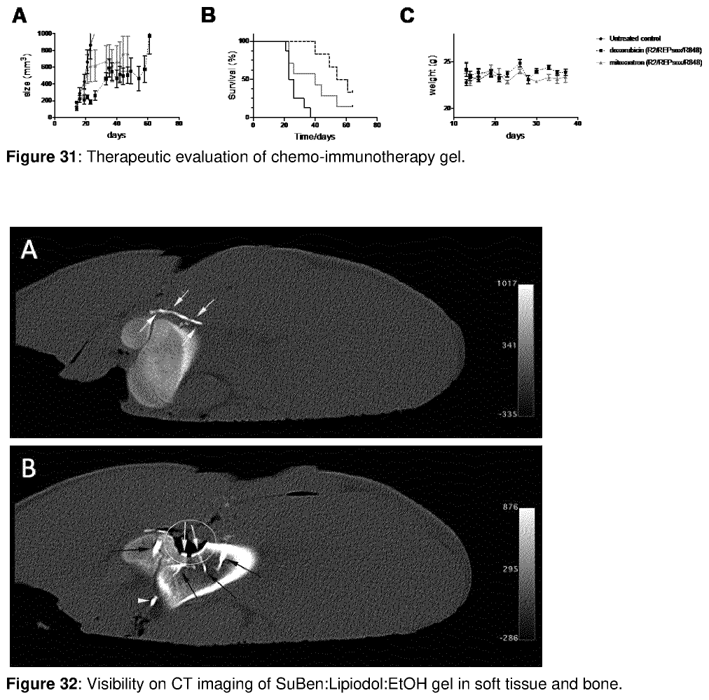

The chemo-immunotherapy gel was highly effective at controlling tumor growth (FIG. 31A) and significantly increased median survival time of treated mice compared to untreated controls (FIG. 31B) (Log-rank; mitoxantrone vs. untreated controls p=0.03, doxorubicin vs. untreated controls, p=0.003). None of the chemo-immunotherapy gel injected mice displayed failure to thrive or problematic weight loss (FIG. 31C). The presented results indicate that chemo-immunotherapy gels can provide a therapeutically effective treatment providing a combined cytotoxic and immune activating effect. These observations indicate that the controlled release chemo-immunotherapy gel formulation may provide the highly attractive multitargeted approach to achieve both cancer cell cytotoxicity and intratumoral immune activation in the hostile immunosuppressive tumor microenvironment.

### Conclusion

The presented example demonstrates that a chemo-immunotherapy gel is well tolerated and is effective in controlling tumor growth and increase median survival of treated mice.

### Example 35: Injectability and Radiographic Visibility of SuBen:Lipiodol:EtOH (60:25:15 (% w/w)) in Soft Tissue and Bone

The aim of the current example is to demonstrate the injectability and visibility on CT imaging of SuBen:Lipiodol:EtOH (60:25:15 (% w/w)) in soft tissue and bone.

### Materials and Methods

SuBen:Lipiodol:EtOH (60:25:15 (% w/w)) was prepared as described in example 19.

Soft tissue injection: The muscular tissue adjacent to the tibia and fibula of standard breed pig carcasses (approx. 40 kg) was injected with approximately 200 μL of SuBen:Lipiodol:EtOH (60:25:15(% w/w)) gel using a 21G/76 mm needle 1 ml syringe. The needle was slowly retracted during the injection to generate an elongated gel depot.

Bone injection: The proximal tibia of standard breed pig carcasses (approx. 40 kg) was surgically approached and a cavity was generated, by sharp curvetting, to mimic the excavation performed in relation to debridement of an osteomyelitis lesion. The intracavitary area was injected (21G/76 mm needle 1 ml syringe) with two separate injections (50 μL-300 μL) of SuBen:Lipiodol:EtOH (60:25:15 (% w/w)) gel. Three holes (2 mm) were drilled in cortical bone surrounding the excavated cavity and a needle (21G/76 mm) advanced in the trabecular bone. Separate injections of 50 μL to 300 μL of SuBen:Lipiodol:EtOH (60:25:15 (% w/w)) gel was performed a multiple sites in the trabecular bone surrounding the excavated cavity. One hour after the injection of SuBen:Lipiodol:EtOH (60:25:15 (% w/w)) legs were CT scanned (Somatom Emotion Siemens, Erlangen, Germany). Images were subjectively assessed for SuBen:Lipiodol:EtOH (60:25:15 (% w/w)) image contrast and ability to form well delineated gel depots in the various tissues injected.

### Results and Discussion

SuBen:Lipiodol:EtOH (60:25:15 (% w/w)) gel was clearly visible in the pen-osseous musculature injected and the gel attained the elongated form pursued during the injection procedure (FIG. 32A). The viscosity and coalescing properties of the SuBen:Lipiodol:EtOH (60:25:15 (% w/w)) may provide a controllable injection technology for generating soft tissue drug depots. In the injected bone and excavated bone cavity the injected gel was found to provide sufficient image contrast for accurate localization (FIG. 32B). The viscosity and injectability of the SuBen:Lipiodol:EtOH (60:25:15 (% w/w)) gel was found to be optimal for generating well defined gel depots at all sites of bone injected.

### Conclusion

SuBen:Lipiodol:EtOH (60:25:15) gel provides acceptable image contrast on CT imaging and the gel-forming properties was able to form well-defined gel depots at all sites injected. Gel formulation with these viscosity and coalescing properties can form well circumscribed drug delivery depot system for controlled drug release in bone and soft tissue.

### Example 36: Synthesis of Carbohydrate Derivatives

The aim of the current example is to describe the synthesis of relevant carbohydrate derivatives.

### Materials and Methods

General experimental conditions: All reactions were carried out under inert atmosphere (N2). Water sensitive liquids and solutions were transferred via syringe. Water used for washing of the isolated products was in all cases MilliQ water. Organic solutions were concentrated by rotary evaporation at 30-80° C. at 200-0 mbar. Thin layer chromatography (TLC) was carried out using aluminum sheets pre-coated with silica 60F (Merck 5554). The TLC plates were inspected under UV light or developed using a cerium ammonium sulphate solution (1% cerium (IV) sulphate (CeSO₄)₂ and 2.5% hexa-ammonium molybdate in a 10% sulfuric acid solution).

Reagents: Dry solvents were purchased from Acros Organics (AcroSeal, extra dry over molecular sieves). All other chemicals were purchased from Sigma Aldrich and were used as received.

Instrumentation: Nuclear Magnetic Resonance (NMR) was conducted on a Bruker Ascend™ 400 MHz—operating at 401.3 MHz for ¹H nmr—with a 5 mm H— Broadband Dual Channel z-gradient Prodigy cryoprobe at 298 K using the residual solvent as internal standard. All coupling constants (J) are expressed in Hz. The FID files were processed in Mnova Suite. In ¹H-NMR spectra of α,β anomeric mixtures, the integral of H-1 of the most abundant anomer (H-1 β), was set to 1.0 (in case the peaks could be clearly resolved from nearby multiplets), and the percentage of each anomeric species was calculated from the integral ratio of H-1 α and H-1 β. MALDI-TOF MS was conducted on a Bruker Autoflex Speed™ mass spectrometer. The matrix used for MALDI-TOF was a mixture of 2,5 dihydroxy benzoic acid (DHB) spiked with sodium trifluoroacetate in ethanol (60 mg/mL). Mass detection of species that could not be detected by MALDI-TOF MS was performed in positive mode on an ESI micrOTOF-Q III (Bruker Daltonics, Bremen, Germany) with a sample flow rate of 180 μL/h. The ions were scanned in the range 50-3,000 m/z with the following settings: Capillary 4,500 V; end plate offset −500 V; nebulizer 0.3 bar; dry gas 4 L/min at 200° C. The instrument was calibrated prior to the analysis using a solution of 2.5 mM NaOH, 2.25 mM formic acid in 90% i-PrOH/water. Preparative flash and prep chromatography was conducted on a Buchi Reveleris™ system generally utilizing Ecoflex 40 g C18 cartridges with a standard gradient of acetonitrile, water and 0.1% Formic acid (FA). Freezedrying was performed on a Cool Safe CS110-4 Pro™ freezedrier, and liquid N₂ was utilized to freeze the samples.

### Experimental Procedure for Synthesis of Long Chain Aliphatic Lactose Esters

β-lactose (5-10 g) was suspended in dry pyridine under inert atmosphere (N₂). Hereafter, isovaleric or hexanoic anhydride (2.2 eq. pr. hydroxyl group) was carefully added. Then, a catalytic amount of DMAP (0.1 eq.) was added. The reactions were heated to 48° C. overnight and thereafter continued for ˜24 hours at room temperature until TLC (10% acetone, toluene) and MALDI-TOF showed complete acylation of the starting materials. The reactions were concentrated under reduced pressure and co-evaporated with toluene. For lactose octahexanoate, there was still a lot of acid byproduct left, therefore the water bath on the rotary evaporator was replaced with an oilbath and the compound was vacuum distilled with fast rotation at starting temperature 150° C. ending at 220° C. The resulting concentrates were dissolved in dichloromethane and washed with NaHCO₃(aq.) (8×) and water (2×). The organic phases were dried with MgSO₄(s), filtered, concentrated under reduced pressure and dried in vacuo.

### α,β Lactose Octaisovalerate

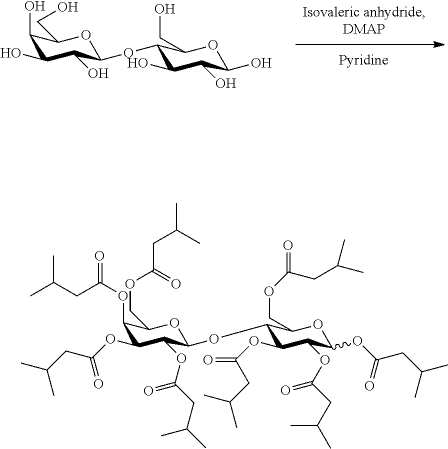

Yield: 52% (˜25% alpha, ˜75% beta anomer). ¹H NMR: ¹H NMR (400 MHz, DMSO-d6) δ 6.15 (d, J=3.7 Hz, 0.3H), 5.88 (d, J=8.3 Hz, 1H), 5.34 (t, J=9.6 Hz, 1H), 5.29-5.25 (m, 1H), 5.25-5.16 (m, 1H), 4.93-4.81 (m, 3H), 4.71 (dd, J=12.7, 8.0 Hz, 1H), 4.37-4.29 (m, 1H), 4.25-4.19 (m, 2H), 4.13-3.96 (m, 5H), 3.91-3.81 (m, 1H), 3.74 (t, J=9.5 Hz, 1H), 2.45-2.06 (m, −20H), 2.02-1.83 (m, −10H), 0.97-0.80 (m, −62H). MALDI TOF-MS: Calculated mass [M+Na]⁺: 1037.58. Found: 1036.77.

### α,β Lactose Octahexanoate

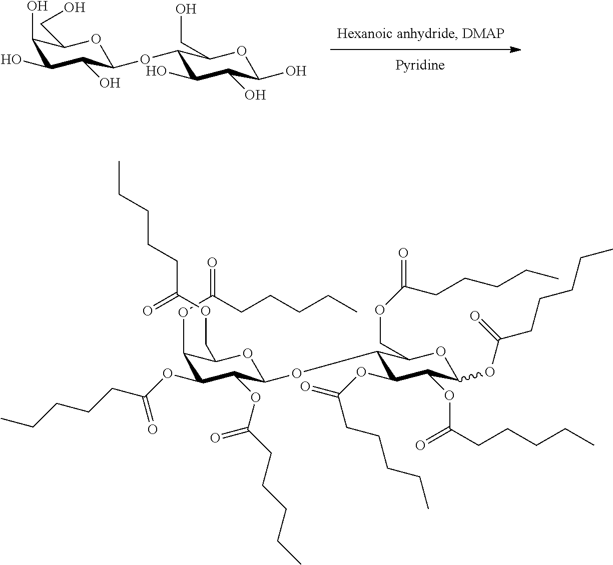

Yield: 66% (˜30% alpha, ˜70% beta anomer). ¹H NMR (400 MHz, DMSO-d₆) δ 6.13 (d, J=3.7 Hz, 0.4H), 5.88 (d, J=8.3 Hz, 1H), 5.34 (t, J=9.5 Hz, 1H), 5.29-5.21 (m, 2H), 5.18 (dd, J=10.3, 3.5 Hz, 1H), 4.91-4.81 (m, 3H), 4.72 (t, J=8.6 Hz, 1H), 4.36-4.27 (m, 1H), 4.25-4.18 (m, 1H), 4.10-4.01 (m, 3H), 4.02-3.96 (m, 3H), 3.94-3.82 (m, 1H), 3.77 (t, J=9.2 Hz, 1H), 2.45-2.07 (m, 22H) (CH2), 1.60-1.37 (m, 22H) (CH2), 1.34-1.13 (m, −44H) (2×CH2), 0.91-0.78 (m, 34H) (CH3). MALDI TOF-MS: Calculated mass [M+Na]⁺: 1149.70. Found: 1148.95.

### Experimental Procedure for Synthesis of Carbohydrate Benzoate Esters

### Sucrose Octabenzoate (SuBen) and Trehalose Octabenzoate (TreBen)

Sucrose or trehalose (10-15 g) was suspended in dry pyridine (200 mL) under inert atmosphere (N2). Hereafter benzoyl chloride (1.2 eq. pr. hydroxyl group) was carefully added over 10 minutes. Then, a catalytic amount of DMAP (0.1 eq.) was added. The reaction was heated to 65° C. overnight, where after MALDI-TOF showed complete acylation of the starting material. The reaction was cooled down to room temperature and a precipitate formed, which was filtered off through suction filtration. The supernatant was then cooled to 2° C. and further precipitate was filtered off, where after the supernatant was concentrated under reduced pressure. The concentrate was dissolved in dichloromethane and washed with NaHCO₃(aq.) (8×150 mL), brine (1×150 mL) and water (1×150 mL). The organic phase was dried with MgSO₄ (s), filtered, concentrated under reduced pressure and dried in vacuo. The solids were recrystallized from dichloromethane:ethanol 1:5 to give white crystalline compounds.

### Sucrose Octabenzoate (SuBen)

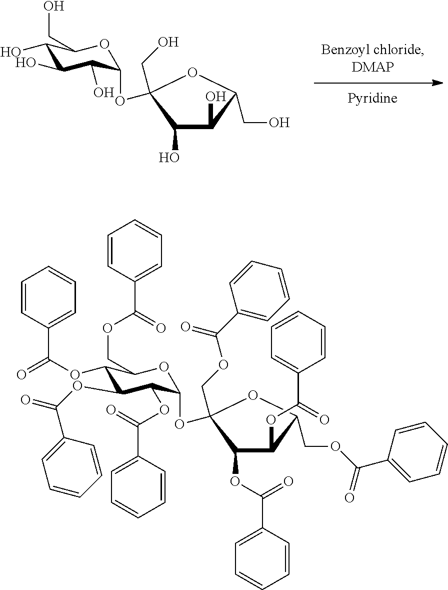

Yield: 53.4%. ¹H NMR (400 MHz, DMSO-d₆) δ 8.20-8.14 (m, 2H), 8.01-7.92 (m, 4H), 7.86 (ddd, J=25.4, 8.2, 1.3 Hz, 4H), 7.77-7.48 (m, 17H), 7.47-7.34 (m, 9H), 7.23 (t, J=7.7 Hz, 2H), 7.08 (t, J=7.7 Hz, 2H), 6.21-6.10 (m, 3H), 5.97 (t, J=6.3 Hz, 1H), 5.83-5.73 (m, 1H), 5.56 (dd, J=10.4, 3.5 Hz, 1H), 4.88-4.79 (m, 2H), 4.75-4.63 (m, 3H), 4.58 (d, J=11.9 Hz, 1H), 4.45 (d, J=3.3 Hz, 2H). MALDI TOF-MS: Calculated mass [M+Na]⁺: 1197.33. Found: 1197.30.

### Trehalose Octabenzoate (TreBen)

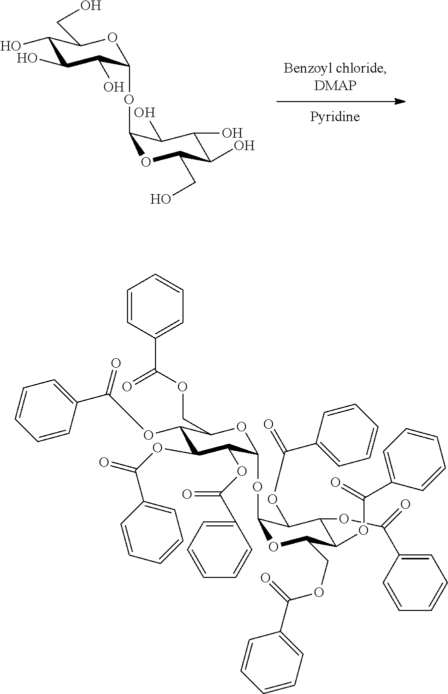

Yield: 44%. ¹H NMR (400 MHz, DMSO-d₆) δ 8.00 (d, J=7.2 Hz, 4H), 7.85 (t, J=8.0 Hz, 8H), 7.68-7.54 (m, 12H), 7.51-7.40 (m, 16H), 6.20 (t, J=9.8 Hz, 2H), 5.82 (d, J=3.7 Hz, 2H), 5.78-5.71 (m, 4H), 4.25 (dt, J=10.2, 3.6 Hz, 2H), 4.18-4.03 (m, 4H). MALDI TOF-MS: Calculated mass [M+Na]⁺: 1197.33. Found: 1197.34.

### α,β Lactose Octabenzoate (LacBen)

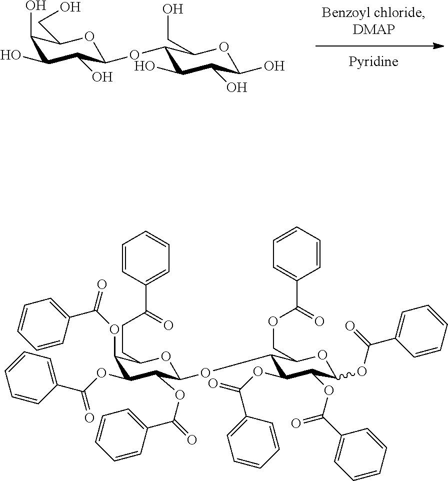

β-lactose (10 g, 29 mmol) was suspended in dry pyridine (150 mL) under inert atmosphere (N2). Hereafter benzoyl chloride (33 mL, 281 mmol, 1.2 eq. pr. hydroxyl group) was carefully added over 10 minutes. Then, a catalytic amount of DMAP (357 mg, 0.1 eq.) was added. The reaction was heated to 65° C. overnight, whereafter MALDI-TOF showed nearly complete acylation. An additional amount of benzoyl chloride was added (4 mL, 34.4 mmol) and the reaction was continued overnight, whereafter MALDI-TOF showed complete acylation. The reaction was cooled down to room temperature and a precipitate was filtered off through suction filtration. The supernatant was then cooled to 2° C. and further precipitate was filtered off, whereafter the supernatant was concentrated under reduced pressure. Half of the crude concentrate was dissolved in toluene and purified by dry column chromatography (acetone in toluene with 0.5-2% increments) and concentrated under reduced pressure. The solid was recrystallized from dichloromethane:ethanol 1:5 to give a white amorphous solid. Yield: 13 g (76%) (˜20% alpha, ˜80% beta). ¹H NMR (400 MHz, DMSO-d₆) δ 8.10-7.79 (m, 14H), 7.77-7.23 (m, 34H), 6.68 (d, J=3.8 Hz, 0.2H), 6.44 (d, J=8.1 Hz, 1H), 6.14-5.99 (m, 1H), 5.86-5.78 (m, 1H), 5.70-5.57 (m, 3H), 5.53-5.33 (m, 2H), 4.68-4.52 (m, 2H), 4.51-4.38 (m, 4H), 3.88 (ddd, J=11.1, 6.7, 4.4 Hz, 1H), 3.69 (dd, J=11.1, 6.8 Hz, 1H). MALDI TOF-MS: Calculated mass [M+Na]⁺: 1197.33. Found: 1197.33.

### Raffinose Undecabenzoate (RaBen)

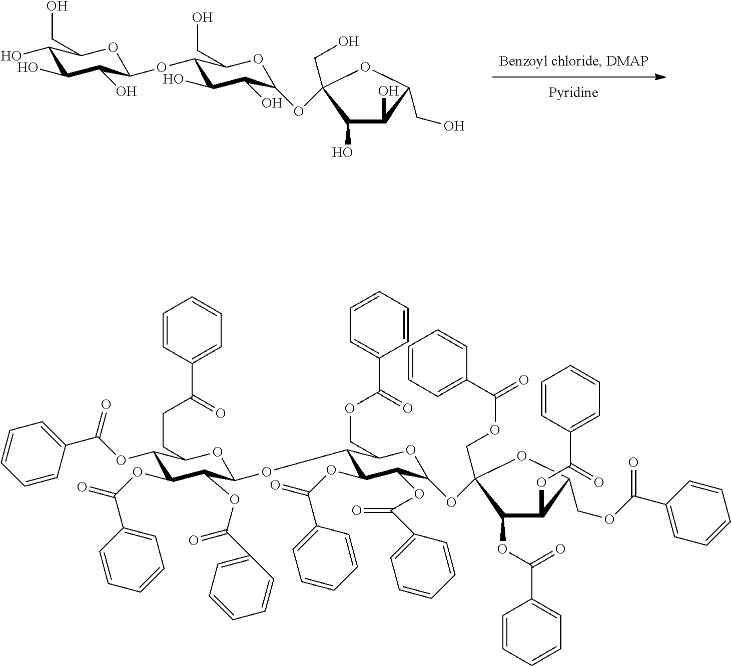

Raffinose pentahydrate (15 g, 25.2 mmol) was suspended in dry pyridine (200 mL) under inert atmosphere (N2). Hereafter benzoyl chloride (39 mL, 333 mmol, 1.2 eq. pr. hydroxyl group) was carefully added over 10 minutes. Then, a catalytic amount of DMAP (308 mg, 0.1 eq.) was added. The reaction was heated to 65° C. overnight, whereafter MALDI-TOF showed a mixture of 5-11 acyl groups, hence another portion of benzoyl chloride (30 mL, 258 mmol) along with 50 mL pyridine was added, and the reaction was continued for another 24 h at 65° C., whereafter MALDI-TOF showed nearly complete acylation of the starting material. The reaction was cooled down to room temperature and a precipitate formed, which was filtered off through suction filtration. The supernatant was then cooled to 2° C. and further precipitate was filtered off, whereafter the supernatant was concentrated under reduced pressure. Part of the crude concentrate (˜10 g) was dissolved in DMSO and purified by automatic flash chromatography (40-100% B over 20 minutes followed by 100% B over 10 minutes, 280 nm, 40 g C18 cartridge) concentrated in vacuo, and freezedried. Yield: 4.11 g (41%). ¹H NMR (400 MHz, DMSO-d₆) δ 8.10 (d, J=7.7 Hz, 2H), 7.98 (dd, J=13.5, 7.7 Hz, 4H), 7.89 (t, J=7.3 Hz, 4H), 7.82 (dd, J=12.4, 7.8 Hz, 4H), 7.77-7.69 (m, 6H), 7.67-7.57 (m, 6H), 7.57-7.48 (m, 9H), 7.48-7.38 (m, 8H), 7.38-7.25 (m, 8H), 7.19 (t, J=7.7 Hz, 2H), 7.08 (t, J=7.7 Hz, 2H), 6.15 (d, J=9.9 Hz, 1H), 6.11 (d, J=5.1 Hz, 1H), 6.04 (t, J=4.1 Hz, 2H), 5.96 (dd, J=10.6, 3.4 Hz, 1H), 5.90 (t, J=5.8 Hz, 1H), 5.76-5.59 (m, 2H), 5.49 (d, J=3.6 Hz, 1H), 5.21 (dd, J=10.4, 3.5 Hz, 1H), 4.90 (q, J=6.0 Hz, 1H), 4.86-4.69 (m, 5H), 4.58 (d, J=12.0 Hz, 1H), 4.39 (d, J=6.3 Hz, 2H), 3.97 (dd, J=11.9, 4.6 Hz, 1H), 3.80 (d, J=11.4 Hz, 1H). MALDI TOF-MS: Calculated mass [M+Na]⁺: 1671.46. Found: 1671.45.

### Experimental Procedure for Synthesis of Glycosides

### Methoxy LOIB (MeLOIB, Methyl Hepta-O-Isobutyryl-α,β-Lactoside)

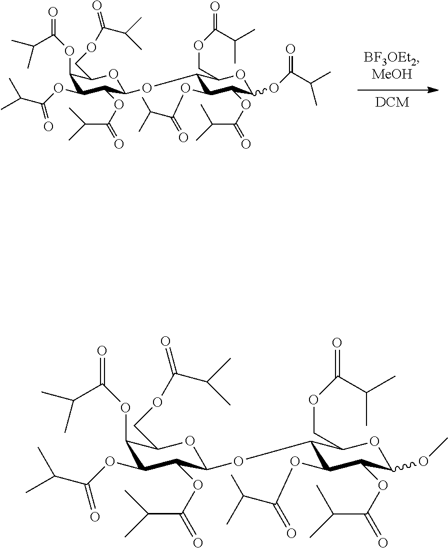

Lactose octaisobutyrate (LOIB, 18 g, 20 mmol) was dissolved in dry dichloromethane (80 mL) under inert atmosphere (N2). Hereafter dry methanol (1.5 mL, 36 mmol, 1.8 eq.) was carefully added, and the reaction was cooled to 0° C. Then, boron trifluoride diethyl etherate (6.2 mL, 50 mmol, 2.5 eq.) was gradually added over 10 minutes. The reaction was allowed to slowly warm to room temperature and continued at room temperature overnight, whereafter MALDI-TOF and TLC (10% acetone, toluene) showed around 65% conversion. In order to avoid excess brown polymerization byproduct, the reaction was stopped by addition of 30 mL methanol. The reaction mixture was washed with NaHCO₃(aq.) (3×100 mL) and water (1×100 mL). The organic phase was dried with MgSO₄ (s), filtered and concentrated under reduced pressure. Part of the crude concentrate (˜12 g) was dissolved in DMSO:MeCN 1:1 and purified by automatic flash (40-80% B over 8 minutes followed by 80-90% B over 17 min, 90-100% B over 2 min and 100% B over 3 minutes, 220 nm, 40 g C18 cartridge) concentrated in vacuo, and freezedried. Yield: 5.2 g (43%). ¹H NMR (400 MHz, DMSO-d₆) δ 5.27-5.09 (m, 3H), 4.89-4.77 (m, 1H), 4.68-4.63 (m, 1H), 4.63-4.59 (m, 1H), 4.29 (d, J=11.5 Hz, 1H), 4.19 (t, J=6.8 Hz, 1H), 4.12 (dd, J=12.1, 6.1 Hz, 1H), 4.05-3.91 (m, 2H), 3.91-3.66 (m, 3H), 3.29 (s, 3H), 2.66-2.22 (m, 7H), 1.10-0.89 (m, ˜42H). MALDI TOF-MS: Calculated mass [M+Na]⁺: 869.42. Found: 869.36.

### 3-Iodobenzyl LOIB (CLA-1,3-Iodobenzyl Hepta-O-Isobutyryl-α,β-Lactoside)

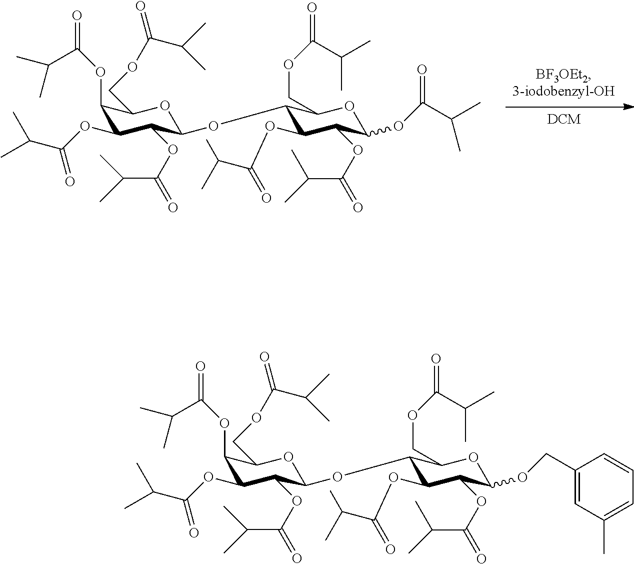

Lactose octaisobutyrate (LOIB, 11 g, 12.2 mmol) was dissolved in dry dichloromethane (60 mL) under inert atmosphere (N2). Hereafter dry 3-iodobenzyl alcohol (containing about 25% impurity of benzyl alcohol) (2.17 mL, 17 mmol, 1.4 eq.) was carefully added, and the reaction was cooled to 0° C. Then, boron trifluoride diethyl etherate (3.8 mL, 30.5 mmol, 2.5 eq.) was gradually added over 10 minutes. The reaction was allowed to slowly warm to room temperature and continued at room temperature overnight, whereafter MALDI-TOF and TLC (10% acetone, toluene) showed around 50% conversion. In order to avoid excess brown polymerization byproduct, the reaction was stopped by addition of 30 mL methanol. The reaction mixture was washed with NaHCO₃(aq.) (3×100 mL) and water (1×100 mL). The organic phase was dried with MgSO₄ (s), filtered and concentrated under reduced pressure. Part of the crude concentrate (˜6 g) was dissolved in DMSO:MeCN 1:1 and purified by automatic flash (40-80% B over 6 minutes followed by 80-90% B over 22 min, 90-100% B over 1 min and 100% B over 5 minutes, 220 and 280 nm, 40 g C18 cartridge) concentrated in vacuo, and freezedried. Yield: 1.6 g (25%). ¹H NMR (400 MHz, DMSO-d₆) δ 7.68-7.60 (m, 2H), 7.26 (d, J=7.7 Hz, 1H), 7.14 (t, J=7.7 Hz, 1H), 5.27-5.15 (m, 3H), 4.91-4.83 (m, 2H), 4.77-4.66 (m, 3H), 4.51 (d, J=12.7 Hz, 1H), 4.35 (dd, J=12.4, 1.9 Hz, 1H), 4.23 (t, J=6.8 Hz, 1H), 4.16 (dd, J=12.1, 5.8 Hz, 1H), 4.08-3.94 (m, 2H), 3.88 (ddd, J=10.0, 5.7, 1.9 Hz, 1H), 3.79 (t, J=9.5 Hz, 1H), 2.69-2.52 (m, 3H), 2.47-2.29 (m, 4H), 1.15-0.95 (m, 42H). MALDI TOF-MS: Calculated mass [M+Na]⁺: 1071.35. Found: 1071.44.

### Experimental Procedure for Synthesis of Iodine Rich Esters

### α,β Lactose Octa Para-Iodobenzoate (CLA-8)

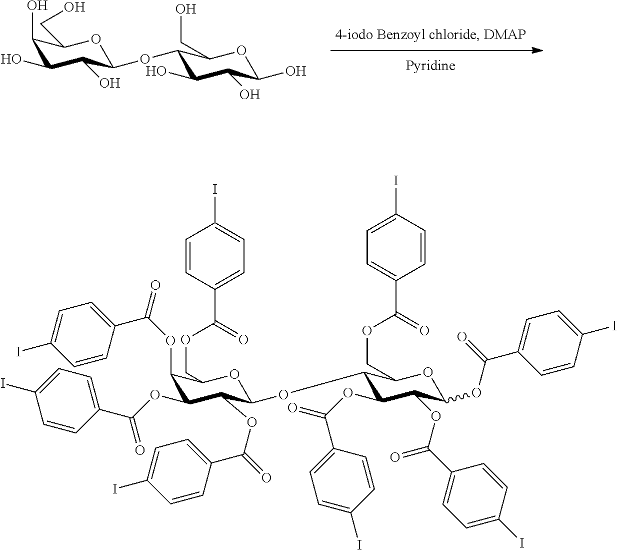

β-lactose (3.4 g, 10 mmol) was suspended in dry pyridine (100 mL) under inert atmosphere (N2). Hereafter para-iodobenzoyl chloride (25 g, 95 mmol, 1.2 eq. pr. hydroxyl group) was carefully added. Then, a catalytic amount of DMAP (121 mg, 0.1 eq.) was added, and the mixture was sonicated under inert atmosphere at 50° C. for 20 minutes for complete dissolution of all reagents. Then the reaction was heated to 65° C. and continued overnight, whereafter TLC (5% acetone, toluene) and QTOF-MS showed complete acylation of the starting material. The reaction was cooled down to room temperature and a precipitate formed, which was filtered off through suction filtration. The supernatant was then cooled to 2° C. and further precipitate was filtered off, whereafter the supernatant was concentrated under reduced pressure. The concentrate was dissolved in dichloromethane and washed with NaHCO₃(aq.) (8×100 mL), brine (1×100 mL) and water (1×100 mL). The organic phase was dried with MgSO₄ (s), filtered, concentrated under reduced pressure and dried in vacuo. The solid was recrystallized from dichloromethane:ethanol 1:5 to give a white crystalline compound. Yield: 12.9 g (60%) (˜25% alpha, ˜75% beta anomer). ¹H NMR (400 MHz, DMSO-d₆) δ 7.99 (d, J=8.4 Hz, 1H), 7.94-7.42 (m, 37H), 7.28 (dd, J=8.6, 2.9 Hz, 3H), 6.60 (d, J=3.7 Hz, 0.3H), 6.40 (d, J=8.0 Hz, 1H), 6.06 (t, J=9.0 Hz, 1H), 5.95 (t, J=9.7 Hz, 0.3H), 5.86-5.72 (m, 1H), 5.64 (dd, J=7.5, 3.5 Hz, 1H), 5.56 (dd, J=10.1, 3.8 Hz, 0.3H), 5.50 (t, J=8.7 Hz, 1H), 5.43-5.31 (m, 3H), 4.69-4.41 (m, 4H), 4.39-4.27 (m, 3H), 3.99 (dd, J=11.2, 6.6 Hz, 1H), 3.87-3.70 (m, 1H). QTOF-MS: Calculated mass [M+Na]⁺: 2204.50. Found: 2204.40.

### α,β Lactose Para-Iodobenzoate, Partly Acylated Mixture

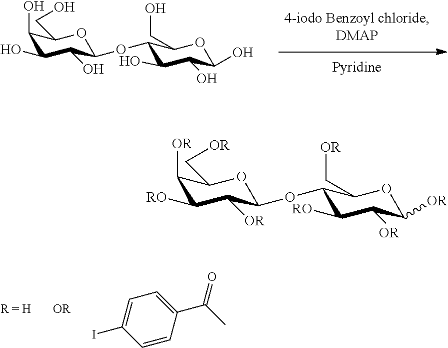

β-lactose (3 g, 9 mmol) was suspended in dry pyridine (100 mL) under inert atmosphere (N₂). Hereafter para-iodobenzoyl chloride (11.2 g, 42 mmol, 1.2 eq. pr 4 hydroxyl groups) was carefully added. Then, a catalytic amount of DMAP (107 mg, 0.1 eq.) was added, and the mixture was sonicated under inert atmosphere at 50° C. for 20 minutes to fully dissolve all constituents. Then the reaction was heated to 65° C. and continued overnight, whereafter MALDI-TOF MS showed conversion to species with 1-5 ester functionalizations. The reaction was then cooled to 2° C. and the resulting precipitate was filtered off on glass filter funnel (por 3), and washed with cold water and ethanol. The precipitate was hereafter recrystallized from isopropanol and collected as a fine white powder with an average of 4 ester functionalizations. Yield: 10.2 g (92%), mixture of partly acylated lactose species. ¹H NMR δ 8.04-7.29 (m, ˜16H), 6.55-3.25 (m, ˜14H). MALDI TOF-MS: Calculated mass: lactose mono para-iodobenzoate [M+Na]⁺: 595.04. Found: 594.84. Lactose di para-iodobenzoate: Calculated mass [M+Na]⁺: 824.96. Found: 824.89. Lactose tri para-iodobenzoate: Calculated mass [M+Na]⁺: 1054.89. Found 1054.87. Lactose tetra para-iodo benzoate Calculated mass [M+Na]⁺: 1284.81. Found: 1284.81. Lactose penta para-iodobenzoate: Calculated mass [M+Na]⁺: 1514.73. Found: 1514.71.

### Example 37: Chemical Structures Beyond CLA-1, CLA8 and meLOIB

The aim of the current example is to present particularly interesting chemical analogues of CLA-1, CLA-8 and meLOIB.

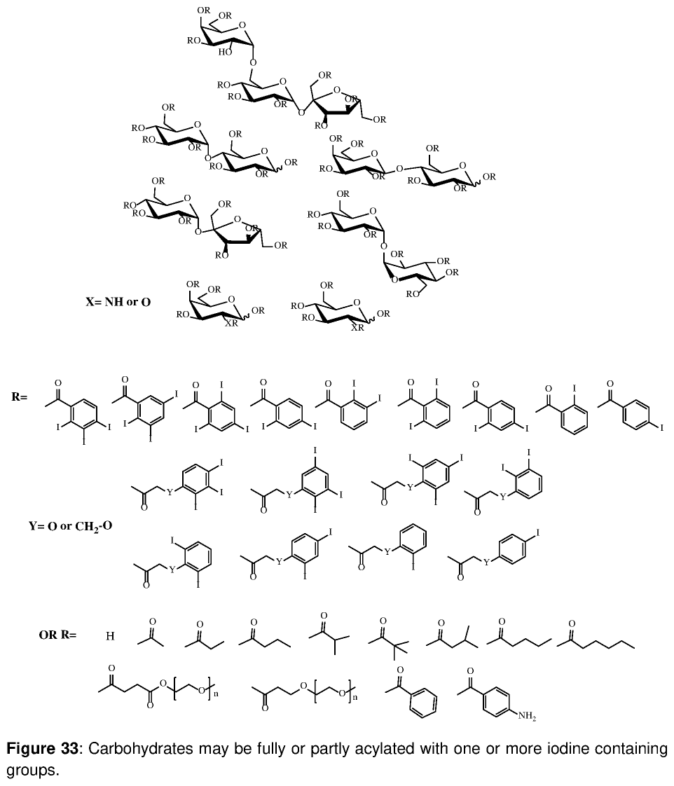

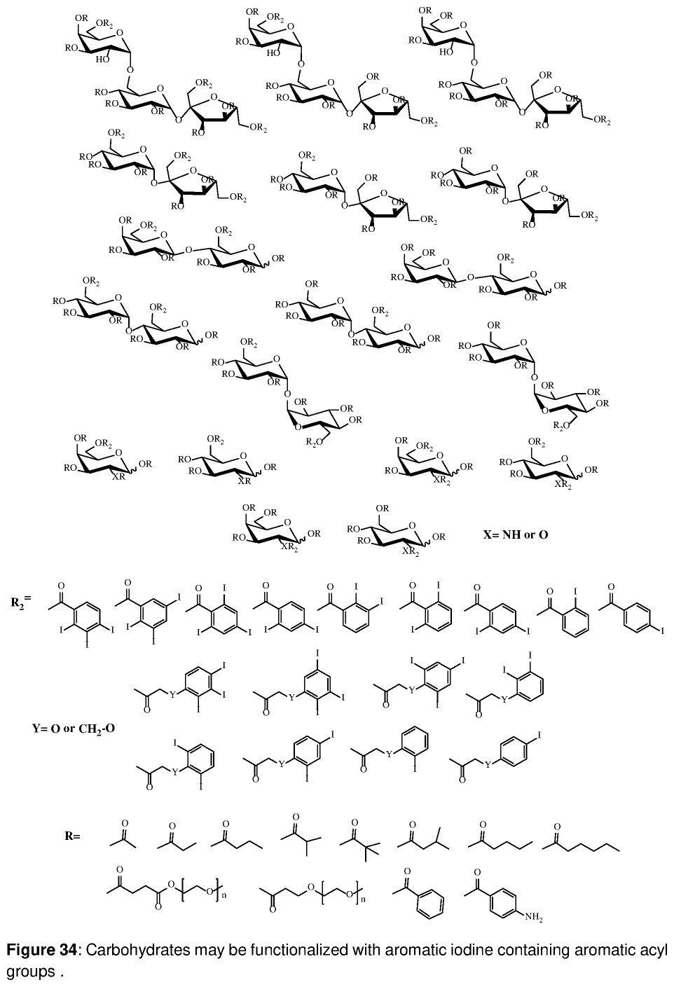

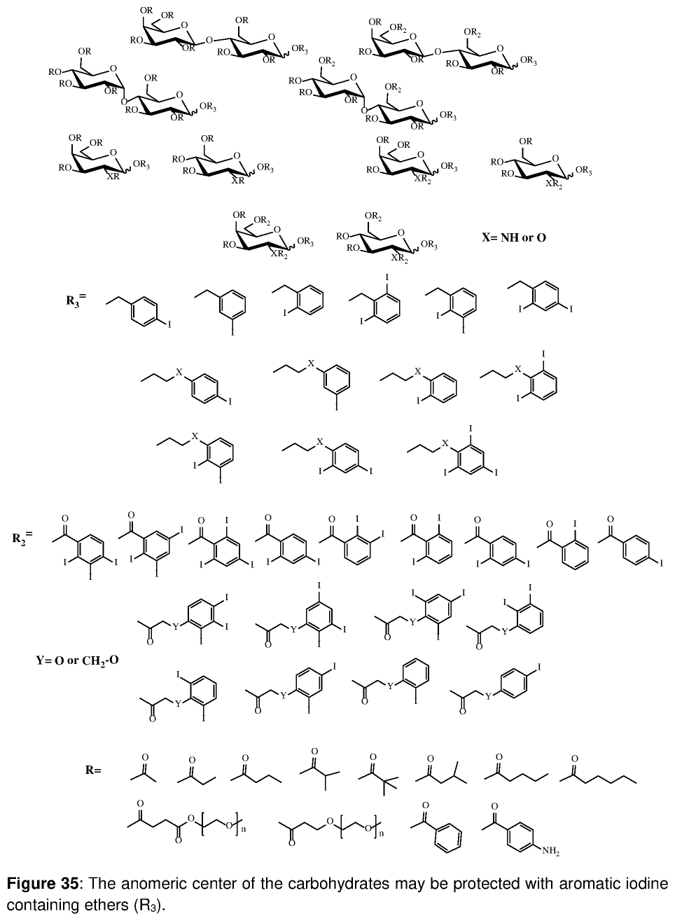

FIG. 33 shows carbohydrates that may be fully or partly acylated with iodine containing aromatic acyl groups or aliphatic or aromatic acyl groups such as acetate, propionate, butyrate, isobutyrate, pivaloate, valerate, isovalerate, hexanoate, benzoate, PABA acylation or PEG acylation or OH.

FIG. 34 shows carbohydrates that may be functionalized with aromatic iodine containing aromatic acyl groups selectively on primary alcohols and/or amines on one or more positions in any acylation pattern, the rest of the positions may be functionalized with one or more aliphatic or aromatic acyl groups in any acylation pattern.

FIG. 35 shows carbohydrates where the anomeric center may be protected with aromatic iodine containing ethers. The primary positions and/or amines may be functionalized with aromatic iodine containing acyl groups on one or more positions. The rest of the positions may be functionalized with one or more of the acyl groups in any pattern.

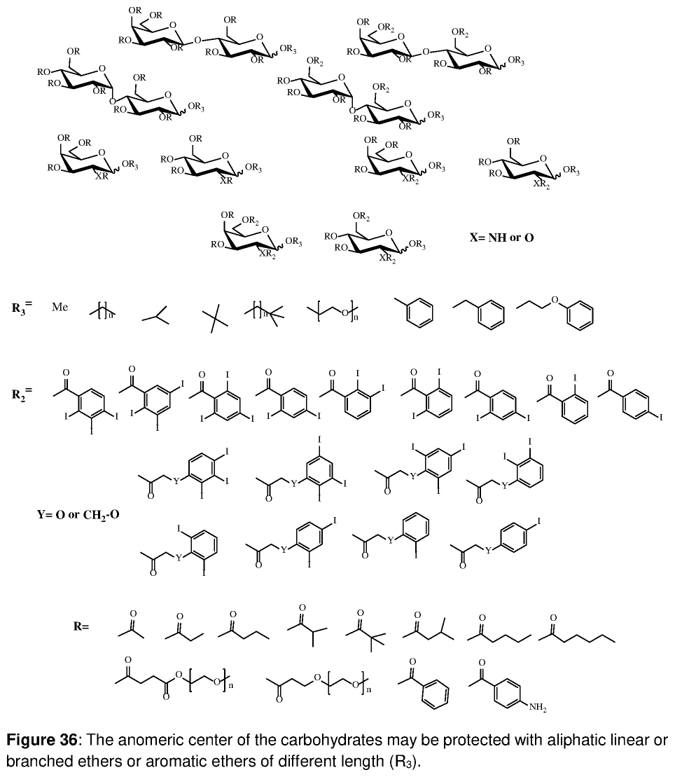

FIG. 36 shows carbohydrates where the anomeric center may be protected with aliphatic linear or branched ethers or aromatic ethers of different length. The primary positions and/or amines may simultaneously be functionalized with aromatic iodine containing acyl groups on one or more positions, the rest of the positions may be functionalized with one or more of the acyl groups in any pattern.

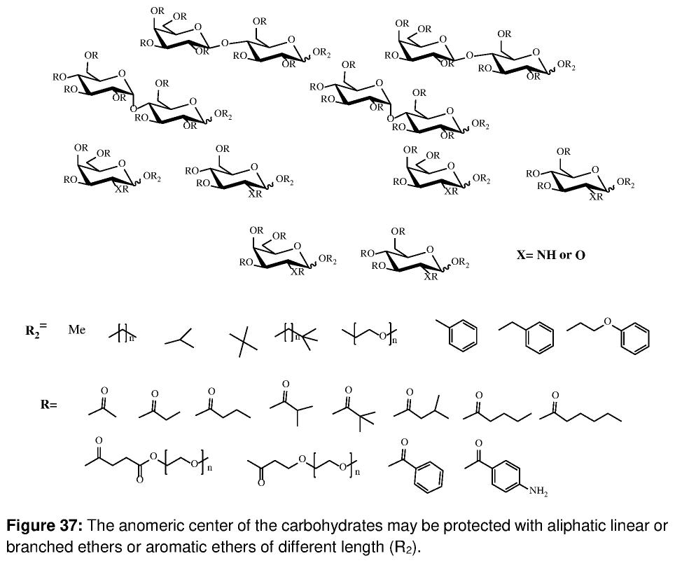

FIG. 37 shows carbohydrates where the anomeric center may be protected with aliphatic linear or branched ethers or aromatic ethers of different length. The rest of the positions may be functionalized with one or more of the acyl groups in any pattern.

1. A composition being homogenous at 20 degrees Celsius, comprising at least:
(a) A hydrophobic disaccharide;
(b) A solvent selected from any of Dimethylsulfoxid, Ethanol (EtOH), Propylenecarbonate, or Benzyl Alcohol, or a combination thereof;
(c) A lipid oil;
(d) An iodinated hydrophobic lipid or iodinated saccharide; and
(e) At least one active pharmaceutically ingredient.

2. The composition according to claim 1, wherein the iodinated hydrophobic lipid or iodinated saccharide is an iodinated disaccharide comprising iodinated benzyl groups or iodinated benzoyl groups covalently bound.

3. The A-composition according to claim 1, wherein the iodinated hydrophobic lipid is the lipid oil Lipiodol.

4. The composition according to claim 1, wherein the hydrophobic disaccharide is an iodinated lactose derivative.

5. The A-composition according to claim 1, which comprises a disaccharide derivative selected from any of Lactose octabenzoate (LacBen), Sucrose octabenzoate (SuBen), Methyl hepta-O-isobutyryl-α,β-lactoside (MeLOIB), α,β-Lactose octa para-iodobenzoate, 3-iodobenzyl hepta-O-isobutyryl-α,β-lactoside (3-iodobenzyl LOIB), or a mixture hereof.

6. The A-composition according to claim 1, which comprises a lipid oil selected from any of glycerol trihexanoate, Glycerol trioctanoate (GTO), Glycerol tridecanoate, ethyl myristate, ethyl palmitate, ethyl oleoate, or mixtures thereof

7. The composition according to claim 1, wherein the composition is a liquid before administration into a human or animal body and increases in viscosity by more than 2,000 centipoise (cP) after administration within 72 hours.

8. The composition according to claim 1, wherein the composition becomes a gel-like material or a solid after administration, such as a crystalline or amorphous solid and remains within 8 cm from the site of injection for at least 2 weeks.

9. The composition according to claim 1, wherein the composition provides an increase in viscosity after administration into the human or animal body which is due to diffusion of the solvent out of the administered composition and into surrounding tissue.

10. The composition according to claim 1, wherein at least 50 (w/w %) of the composition is a hydrophobic dissacharide.

11. The composition according to claim 1, which further contains 0.1%-5% (w/w %) poly lactic acid (PLA) or poly(lactic-co-glycolic acid) (PLGA).

12. The composition according to claim 1, wherein the solvent constitutes 4-16% (w/w %) of the composition.

13. The composition according to claim 1, wherein the gel composition is comprised of one of the following compositions: meLOIB:GTO:DMSO, SuBen:GTO:EtOH, LacBen:Lipiodol:EtOH, LacBen:Ethyl-palmitate:EtOH, CLA-8:SuBen:GTO:EtOH, and wherein one or more active pharmaceutical ingredients are dissolved.

14. The composition according to claim 1, wherein the gel composition is comprised of one of the following compositions: meLOIB:GTO:DMSO (82.5:7.5:10 w/w), SuBen:GTO:EtOH (60:25:15 w/w), LacBen:Lipiodol:EtOH (60:25:15 w/w), LacBen:Ethyl-palmitate:EtOH (60:25:15), CLA-8:SuBen:GTO:EtOH (20:40:25:15), and wherein one or more active pharmaceutical ingredients are dissolved.

15. The composition according to claim 1, wherein release of one or more active pharmaceutical ingredients after injection into the animal or human body is controlled by the hydrophobicity of the composition

16. The composition according to claim 1, wherein the active pharmaceutical ingredients are chemically stable for more than 7 days at 20 degrees Celsius, preferably wherein less than 10% of the pharmaceutical ingredient changes chemical structure within 7 days at 20 degrees Celsius, more preferably less than 5% of the pharmaceutical ingredient changes chemical structure within 7 days at 20 degrees, and most preferably less than 1% of the pharmaceutical ingredient changes chemical structure within 7 days at 20 degrees.

17. The composition according to claim 1, wherein one or more of the active pharmaceutical ingredients is a drug that modulates an immune response.

18. The composition according to claim 1, wherein the active pharmaceutical ingredient is a TLR7 or TLR8 agonist, or wherein the active pharmaceutical ingredient is a TLR7 or TLR8 agonist and combined with at least one more active pharmaceutical ingredient that modulates an immune response.

19. The composition according to claim 1, wherein at least one active pharmaceutical ingredient selected from any of a class of TGFβ inhibitors, IFN agonists, IDO inhibitors GSK inhibitors, RIG-I agonists, SHP inhibitors, SHP2 inhibitors, ICD inducers, Sting agonists, PD1 or PD-L1 inhibitors, CTLA4 inhibitors, OX40 agonist, CD40 agonist, CD137 agonist, GITR agonist, TNFSFR agonist or WNT/β-catenin inhibitors is dissolved.

20. The composition according to claim 1, wherein at least one active pharmaceutical ingredient is selected from any of the therapeutic agents Gardiquimod, Resiquimod (R848), Imiquimod, Repsox, Galunisertib, SD-208, NLG919, R08191, CHIR99021, PD0325901, TWS119, AR-A014418, SB415286, GSK-3 inhibitor-X, SHP099, PC-61275, Mitoxantrone, Bortezomib, Crizotinib, doxorubicin, XAV939, KIN1400, KIN1408, or Erythromycin, or a combination thereof.

21. The composition according to claim 1, wherein the active pharmaceutical ingredients are a TLR7 agonist and a TGFβ inhibitor.

22. The composition according to claim 1, wherein the active pharmaceutical ingredients are a RIG-1 agonist and a TGFβ inhibitor.

23. The composition according to claim 1, wherein the active pharmaceutical ingredients are a TLR7 agonist and a GSK inhibitor or a RIG-1 agonist and a GSK inhibitor.

24. The composition according to claim 1, wherein the active pharmaceutical ingredients are a TLR7 agonist and a WNT/β-catenin inhibitor or a RIG-1 agonist and a WNT/β-catenin inhibitor.

25. The composition according to claim 1, wherein the active pharmaceutical ingredients are a TLR7 agonist and a RIG-1 agonist.

26. The composition according to claim 1, wherein the active pharmaceutical ingredients are a TLR7 agonist and a TNFSFR agonist or a RIG-1 agonist and a TNFSFR agonist.

27. The composition according to claim 1, wherein the active pharmaceutical ingredients are a TLR7 agonist and a SHP2 inhibitor or a RIG-1 agonist and a SHP2 inhibitor.

28. The composition according to claim 1, wherein at least one of the active pharmaceutical ingredients is dissolved at a concentration higher than 1 mg/g of composition, preferably at least 2 mg/g of composition, more preferably at least 5 mg/g of composition, most preferably at least 10 mg/g composition at 20 degrees Celcius.

29. The composition according to claim 1, wherein the active pharmaceutical ingredients are a cytotoxic agent and one or more pharmaceutical ingredients that modulates an immune response.

30. The composition according to claim 1, wherein the active pharmaceutical ingredients are an ICD inducer and a TLR7 agonist or an ICD inducer and a RIG-1 agonist.

31. The composition according to claim 1, wherein the active pharmaceutical ingredients are an ICD inducer and a TLR7 agonist and one immune modulating agent selected from any of TGFβ inhibitors, GSK inhibitors, SHP2 inhibitors, WNT/β-catenin inhibitors, OX40 agonist, CD40 agonist, CD137 agonist, GITR agonist, RIG-1 or TNFSFR agonist.

32. The composition according to claim 1, wherein the active pharmaceutical ingredients are an ICD inducer and a RIG-1 agonist and one immune modulating agent selected from any of TGFβ inhibitors, GSK inhibitors, SHP2 inhibitors, WNT/β-catenin inhibitors, OX40 agonist, CD40 agonist, CD137 agonist, GITR agonist or TNFSFR agonist.

33. The composition according to claim 1, wherein the active pharmaceutical ingredients are released from the composition after administration to a human or animal body at comparable rates.

34. The composition according to claim 1, wherein the active pharmaceutical ingredients are released from the composition after administration to a human or animal body at substantially different rates.

35. The composition according to claim 1, wherein the composition comprises contrast agents that make the composition visible in one or more imaging modalities such as ultrasound imaging, CT imaging, x-ray imaging, fluoroscopy imaging, fluorescence imaging, MR imaging or OCT imaging.

36. A method of administering a composition according to claim 1 into diseased tissue of a human or animal body, wherein the composition is administered through a hypodermic needle, pig-tail catheter, intravascular catheter, endoscopy aspiration needle, bone marrow aspiration needle and a syringe, an endoscope, a bronchoscope, bone marrow injection device, or stereotactic injection frame under image guidance.

37. The method according to claim 36, wherein a CT, fluoroscopy, ultrasound, OCT or x-ray image is recorded after administration to verify placement of the composition within the diseased tissue.
